(19) United States
(12) Patent Application Publication
(10) Pub. No.: US 2011/0052650 A1
(43) Pub. Date: Mar. 3, 2011

(54) POROUS CRYSTALLINE HYBRID SOLID FOR ADSORBING AND RELEASING GAS OF BIOLOGICAL INTEREST

(75) Inventors: Russel Morris, Gauldry (GB); Christian Serre, Plaisir (FR); Patricia Horcajada Cortes, Trappes (FR); Alexandre Vimont, Merville-Franceville (FR); Thomas Devic, Villebon-Sur-Yvette (FR); Gérard Ferey, Paris (FR)

(73) Assignee: Centre National De La Recherche Scientifique - CNR S, Paris Cedex 16 (FR)

(21) Appl. No.: 12/935,642

(22) PCT Filed: Mar. 31, 2009
(86) PCT No.: PCT/FR09/00381

§ 371 Date: Nov. 18, 2010

(30) Foreign Application Priority Data
Apr. 1, 2008 (FR) 0852150
Jun. 10, 2008 (FR) 0803214

(51) Int. Cl. A61K 8/02; A61K 9/00; A61K 33/00; A61K 8/58; A01N 25/08; A61K 8/73; A01N 59/00; A61P 17/00; A61P 9/10; A01P 1/00; A61P 31/04

(52) U.S. Cl. 424/401; 424/400; 424/718; 424/699; 424/409

(57) ABSTRACT

The invention relates to solids made of a porous crystalline metal-organic framework (MOF) loaded with at least one gas of biological interest, and to a method for preparing the same. The MOF solids of the present invention are capable of adsorbing and releasing in a controlled manner gases having a biological interest. They can be used in the pharmaceutical field and/or for applications in the cosmetic field. They can also be used in the food industry.

### TECHNICAL FIELD

The present invention relates to solids made up of a porous crystalline metal-organic framework (MOF) loaded with at least one gas of biological interest, and also to the method for preparing same.

The MOF solids of the present invention are capable of adsorbing and releasing gases of biological interest in a controlled manner. They can be used in the pharmaceutical field and/or for applications in the cosmetics field. They can also be used in the food industry.

The references between square brackets [X] refer back to the list of references at the end of the examples.

### PRIOR ART

Metal-organic frameworks (MOFs) are coordination polymers with an inorganic-organic hybrid frame comprising metal ions and organic ligands coordinated with the metal ions. These materials are organized in a one-, two- or three-dimensional framework in which the metal clusters are linked to one another periodically by spacer ligands. These materials have a crystal structure, are most commonly porous and are used in many industrial applications such as gas storage, liquid adsorption, liquid or gas separation, catalysis, etc.

Mention may be made, for example, of patent application U.S. Ser. No. 10/039,733 [1] which describes a reaction process involving a catalytic system comprising a zinc-based MOF material. This same material is also used for gas storage in patent application U.S. Ser. No. 10/061,147 [2].

In addition, MOF materials based on frameworks of the same topology are described as “isoreticular”. These spatially organized frameworks have made it possible to obtain a more homogeneous porosity. Thus, patent application U.S. Ser. No. 10/137,043 [3] describes several zinc-based IRMOF (isoreticular metal-organic framework) materials used for gas storage.

Moreover, gases of biological interest, such as NO, CO and H₂S, are extremely important for the biological functioning of mammals. They are involved in a large number of processes, for instance vasodilation, the prevention of platelet aggregation and thrombosis formation, neurotransmission and wound healing.

It is known that both carbon monoxide (CO) and nitric oxide (NO) at very low concentrations have an important activity as signaling molecules in the body.

NO has been thoroughly studied in biology [32]. The biological activity of NO comprises [33]:
anti-inflammatory activity,
regulation of sexual dysfunction,
cardiovascular indications (treatment of angina pectoris).

Furthermore, NO is involved in the activity of many medicaments (calcium channel blocker, ACE inhibitors and ANGII receptor type 1 antagonists, β-blocker and hydroxymethylglutaryl-CoA reductase inhibitors).

As regards CO, even though the mechanism of action is yet to be elucidated for CO, its involvement in many physiological effects is known and described [34].

Thus, CO is involved in biological activities such as, for example:
1. anti-inflammatory activity
   reduces endotoxic shock [35],
   reduces allergic inflammation [36];
2. suppression of the rejection of transplanted organs [37];
3. protection against hyperoxia [38];
4. protection against ischemia [39];
5. protection of beta-pancreatic cells against apoptosis [40];
6. modulation of spermatogenesis under conditions of stress through Leydig cells [41];
7. decrease in perfusion pressure in the isolated regions of the human placenta [42];
8. protection against septic shock and pulmonary lesions in animal models,
9. modulation of vascular smooth muscle tonus [44];
10. regulation of arterial pressure in a situation of stress [45];
11. suppression of arteriosclerotic lesions associated with chronic diseases and transplant rejection [46].

Scientific research studies on the role of CO emissions in the organism are still at an early stage. There are research studies which imply that CO may also be significantly involved in other fields of medicine, including transplant surgery, neuroprotection in strokes, and Alzheimer's disease [47]. CO is also implicated in the control of placenta vascular function [48].

The beneficial role of CO in the organism has also been described in three patent applications: US patent 2002155166, WO 0278684 and WO 02092075. These applications describe the use of CO gas in the medical field. US 2002155166 describes, for example, the use of CO as a biological marker and therapeutic agent for various types of diseases and in the transplantation field. WO 0278684 relates to the methods and the compositions for treating vascular, inflammatory and immune system diseases, using compounds capable of generating CO in vivo. CH₂Cl₂ is described as being the preferred compound capable of generating CO in vivo. This is because CH₂Cl₂ is metabolized to CO and thus provides a source of gas. WO 02092075 uses metal carbonyls as the source of CO gas emissions.

The value of H₂S as a signaling molecule is becoming increasing great [49]. Since the pKa of H₂S is 6.8, at physiological pH it is especially present as H₂S and [HS]₂. The concentration of H₂S is 50-160 mM in brain tissue and 10-100 mM in the blood.

H₂S is active in the cardiovascular system and in the central nervous system. For example, it can cause vasodilation [50]. Moreover, it is capable of readily coordinating heme groups and certain cytochromes. More research is necessary to demonstrate the importance of H₂S as a signaling molecule.

The use of these exogenous gases opens up a very large number of possibilities and said uses have become important stakes in the development, in particular, of new medicaments or prophylactic and therapeutic methods including potential applications in anti-thrombogenic methods, improved efficacy of the treatment of wounds and ulcers, the treatment of fungal and bacterial infections, etc. The controlled release of this type of gas, in particular NO, by virtue of its antibacterial properties, could also prove to be useful for applications in cosmetics, in particular for cosmetic creams [4], but also for food preservation (antibacterial and antioxidant effect) [5].

In the particular case of NO, the homogeneous delivery of this gas from a solution is already known for certain pathological conditions (i.e. from glyceryl trinitrate for treating angina). However, this approach is restricted because of adverse side effects as a consequence of the large variety of effects that it has depending on the location (vasodilator and inhibitor of platelet aggregation, endothelium; microbicide, macrophages, in some circumstances NO can cause harmful side effects: this is the case in septicemia, where the excessive production of NO by the macrophages results in massive vasodilation, the main cause of hypertension encountered in septic shock; neurotransmitter, nerve cells; smooth muscle relaxant, digestive tube; regulator of apoptosis, antiapoptotic or apoptotic depending on the presence or absence of cellular reducing agents).

The inhalation of NO gas has also been used for treating certain pathological lung conditions. However, the delivery of NO in gas form from gas bottles is not practical and limits the value of such a method.

Moreover, a significant proportion of the therapies linked to the gas of biological interest requires a controlled and targeted release of said gas in specific areas of the human body, thus avoiding systemic effects [6]. In the particular case of NO, this is very important since NO has a short biological lifetime.

The current technologies have many drawbacks. For example, polymers of NONOate type or with a metal component have a low storage capacity, requiring a high pressure for loading the NO, and are relatively expensive and potentially toxic [7, 8].

In dermatological applications, acid creams based on nitrates, which are potential NO donors, are pro-inflammatory and are not suitable for sensitive skin [9].

Recently, the use of zeolite-type porous solids or of hybrid solids for NO storage has been described [10, 11, 12]. These solids have a storage capacity which surpasses that of the other materials, a long lifetime (the capacity for NO release after 2 years of storage remains intact), are inexpensive and do not appear to exhibit any toxicity, thus making them very attractive.

Furthermore, the dermatological tests have shown that NO-loaded zeolites are compatible with human skin, including sensitive skin [13].

Despite the abovementioned advantages, the delivery of NO with zeolites can take place only over a short period of time, thus making them unsuitable for an application for which long-lasting release is desired.

The adsorption, storage and release of NO by copper-based or aluminum-based porous metal-organic frameworks for inhibiting platelet aggregation have also been described. Despite the large NO adsorption and storage capacity of these solids (adsorption significantly improved compared with other solids such as organic polymers or zeolites), once they are in contact with a biological/physiological solution (platelet-rich plasma), these solids show poor stability.

As mentioned, one of the particularly important applications in the field of the release of gases of biological interest, and in particular of NO, concerns antithrombotic equipment such as, for example, stents, catheters and cannulas which are inserted into the blood stream with varying durations for therapeutic or diagnostic purposes, and also extracorporeal circuits used for kidney dialysis and in surgery.

Specifically, the prevention of thromboses is vitally important after the insertion of stents, catheters, prosthetic conduits and other medical implants in the body during the surgical procedure, which can often result in dangerous complications, in particular owing to blood vessel occlusion.

Among the drawbacks of the already known systems, mention may be made, for example, of:
a lack of targeted release of the gases, which results in numerous adverse side effects;
amount released is poorly controlled, and therefore potentially unsuitable for the required application,
duration of release of the gases in a physiological medium is short or accompanied by release of unwanted substances, and/or
poor stability in a biological/physiological medium, thus limiting their use in such media.

There therefore exists a real need to develop systems enabling the adsorption and the release of gases of biological interest, in which said systems have a strong adsorption capacity and the release of the gases can be carried out continuously and in a targeted manner.

Furthermore, there exists a real need to have systems which make it possible to deliver, in a controlled manner, the optimum amount of gas necessary for a given application.

In addition, there exists a real need to have systems which make it possible to control the duration of release of the gases in a physiological medium, in particular so as to be able to have sustained releases that can be for more than 6 hours, while at the same time preserving their stability in a biological/physiological medium throughout this release.

Furthermore, there exists a real need to have systems which have sufficient load capacities, especially if repeated gas administrations are envisaged.

### DESCRIPTION OF THE INVENTION

The aim of the present invention is precisely to satisfy these needs and drawbacks of the prior art by providing porous crystalline MOF solids loaded with at least one Lewis base gas chosen from the group comprising NO, CO and H₂S, at least one part of which coordinates with M, said solid comprising a three-dimensional succession of units corresponding to the following formula (I):

MₘOₖXₗLₚ (1)

in which:
each occurrence of M, which may be identical or different, independently represents an ion of a transition metal M^{z+} chosen from the group comprising Fe, Ti, Zr and Mn and in which z is from 2 to 4, or a mixture thereof;
m is 1 to 12;
k is 0 to 4;
l is 0 to 18;
p is 1 to 6;
X is an anion chosen from the group comprising OH⁻, Cl⁻, F⁻, I⁻, Br⁻, SO₄²⁻, NO₃⁻, ClO₄⁻, PF₆⁻, BF₄⁻, R¹—(COO)ₙ⁻ where R is as defined below, R¹—(COO)ₙ⁻, R¹—(SO₃)ₙ⁻, R¹—(PO₃)ₙ⁻, where R¹ is a hydrogen, an optionally substituted, linear or branched C₁ to C₁₂ alkyl, or an aryl, and where n is an integer from 1 to 4;
L is a spacer ligand comprising a radical R

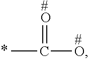

comprising q carboxylate groups where
   q is 1, 2, 3, 4, 5 or 6; *denotes the point of attachment of the carboxylate with the R radical;
   # denotes the possible points of attachment of the carboxylate to the metal ion;
   R represents:
(i) a O₁₋₁₂ alkyl, C₂₋₁₂ alkene or C₂₋₁₂ alkyne radical;
(ii) a fused or nonfused, monocyclic or polycyclic aryl radical containing 6 to 50 carbon atoms;
(iii) a fused or nonfused, monocyclic or polycyclic heteroaryl containing 1 to 50 carbon atoms;
(iv) an organic radical comprising a metal element chosen from the group comprising ferrocene, porphyrin and phthalocyanin;
the R radical being optionally substituted with one or more R² groups, independently chosen from the group comprising C₁₋₁₀ alkyl; C₂₋₁₀ alkene; C₂₋₁₀ alkyne; C₃₋₁₀ cycloalkyl; C₁₋₁₀ heteroalkyl; C₁₋₁₀ haloalkyl; C₆₋₁₀ aryl; C₃₋₂₀ heterocyclic; (C₁₋₁₀)alkyl(C₆₋₁₀)aryl; (C₁₋₁₀)alkyl(C₃₋₁₀)heteroaryl; F; Cl; Br; I; —NO₂; —CN; —CF₃; —CH₂CF₃; —OH; —CH₂OH; —CH₂CH₂OH; —NH₂; —CH₂NH₂; —NHCHO; —COOH; —CONH₂; —SO₃H; —CH₂SO₂CH₃; —PO₃H₂; or a -GR^{G1} function in which G is —O—, —S—, —NR^{G2}—, —C(═O)—S(═O)—, —SO₂—, —C(═O)O—, —C(═O)NR^{G2}—, —OC(═O)—NR^{G2}C(═O)O—, —OC(═O)O—, —OC(═O)NR^{G2}—, —NR^{G2}C(═O)O—, —NR^{G2}C(═O)NR^{G2}— or —C(═S)—, where each occurrence of R^{G2} is, independently of the other occurrences of R^{G2}, a hydrogen atom; or a linear, branched or cyclic, optionally substituted, C₁₋₁₂ alkyl, C₁₋₁₂ heteroalkyl, C₂₋₁₀ alkene or C₂₋₁₀ alkyne function; or a C₆₋₁₀ aryl, O₃₋₁₀ heteroaryl, C₅₋₁₀ heterocyclic, (C₁₋₁₀)alkyl(C₆₋₁₀)aryl or (C₁₋₁₀)alkyl(C₃₋₁₀)heteroaryl group in which the aryl, heteroaryl or heterocyclic radical is optionally substituted; or else, when G represents —NR^{G2}—, R^{G1} and R^{G2}, together with the nitrogen atom to which they are bonded, form a heterocycle or a heteroaryl which is optionally substituted.

The MOFs according to the invention have, inter alia, the advantage:
of being based on nontoxic metals and therefore suitable for an application in the pharmaceutical, medical and/or cosmetics fields,
of having a stability greater than that described for metals such as copper or aluminum,
of having a stability that can be modulated according to the choice of the structure and of the organic ligand, thus making it possible to adapt the MOFs to the various applications desired.

In the context of the present invention, the terms “releasing/release” and “delivering/delivery” will be used without distinction to signify that the gases present in the MOF solids are partially or completely given off.

The term “partially” is intended to mean a release of less than 100% of the amount initially adsorbed.

The term “substituted” denotes, for example, the replacement of a hydrogen radical in a given structure with an R² radical as defined above. When more than one position can be substituted, the substituents may be the same or different at each position.

For the purpose of the present invention, the term “spacer ligand” is intended to mean a ligand (including, for example, neutral species and ions) coordinated with at least two metals, participating in the distancing between these metals and in the formation of empty spaces or pores. The spacer ligand can comprise 1 to 6 carboxylate groups, as defined above, which can be monodentate or bidentate, i.e. comprise one or two points of attachment to the metal. The points of attachment to the metal are represented by the abbreviation # in the formulae. When the structure of a function A comprises two points of attachment #, this means that the coordination to the metal can take place via either or both of the points of attachment.

For the purpose of the present invention, the term “alkyl” is intended to mean a saturated or unsaturated, linear, branched or cyclic, optionally substituted carbon-based radical containing 1 to 12 carbon atoms, for example 1 to 10 carbon atoms, for example 1 to 8 carbon atoms, for example 1 to 6 carbon atoms.

For the purpose of the present invention, the term “alkene” is intended to mean an alkyl radical, as defined above, having at least one carbon-carbon double bond.

For the purpose of the present invention, the term “alkyne” is intended to mean an alkyl radical, as defined above, having at least one carbon-carbon triple bond.

For the purpose of the present invention, the term “aryl” is intended to mean an aromatic system comprising at least one ring which satisfies Hückel's rule for aromaticity. Said aryl is optionally substituted and can contain from 6 to 50 carbon atoms, for example 6 to 20 carbon atoms, for example 6 to 10 carbon atoms.

For the purpose of the present invention, the term “heteroaryl” is intended to mean a system comprising at least one aromatic ring having from 5 to 50 ring members, among which at least one member of the aromatic ring is a heteroatom, in particular chosen from the group comprising sulfur, oxygen, nitrogen and boron. Said heteroaryl is optionally substituted and can contain from 1 to 50 carbon atoms, preferably 1 to 20 carbon atoms, preferably 3 to 10 carbon atoms.

For the purpose of the present invention, the term “cycloalkyl” is intended to mean a saturated or unsaturated, optionally substituted, cyclic carbon-based radical which can contain 3 to 20 carbon atoms, preferably 3 to 10 carbon atoms.

For the purpose of the present invention, the term “haloalkyl” is intended to mean an alkyl radical as defined above, said alkyl system comprising at least one halogen.

For the purpose of the present invention, the term “heteroalkyl” is intended to mean an alkyl radical as defined above, said alkyl system comprising at least one heteroatom, in particular chosen from the group comprising sulfur, oxygen, nitrogen and boron.

For the purpose of the present invention, the term “heterocycle” is intended to mean a saturated or unsaturated, optionally substituted, cyclic carbon-based radical comprising at least one heteroatom and which can contain 2 to 20 carbon atoms, preferably 5 to 20 carbon atoms, preferably 5 to 10 carbon atoms. The heteroatom can, for example, be chosen from the group comprising sulfur, oxygen, nitrogen and boron.

For the purpose of the present invention, the terms “alkoxy”, “aryloxy”, “heteroalkoxy” and “heteroaryloxy” are intended to mean, respectively, an alkyl, aryl, heteroalkyl and heteroaryl radical bonded to an oxygen atom.

For the purpose of the present invention, the terms “alkylthio”, “arylthio”, “heteroalkylthio” and “heteroarylthio” are intended to mean, respectively, an alkyl, aryl, heteroalkyl and heteroaryl radical bonded to a sulfur atom.

The Lewis gases according to the invention are gases of biological interest, chosen from the group comprising NO, CO and H₂S. Said gas is preferably NO.

The particular crystal structure of the MOF solids according to the invention provides these materials with specific properties.

In the MOF solids of the invention, M is chosen from the group comprising Fe, Ti, Mn and Zr. M can also be a mixture of these metals. M is advantageously Fe.

As indicated above, M is a transition metal ion M^{z+} in which z is from 2 to 4. When M is a mixture of metals, for each metal, z can have an identical or different value.

In one embodiment of the invention, the solids of the invention comprise a three-dimensional succession of units of formula (I) in which M can represent a single type of ion M^{z+}, for example Fe, in which z may be identical or different, for example 2 or 3 or a mixture of 2 and 3.

In another embodiment of the invention, the solids of the invention can comprise a three-dimensional succession of units of formula (I) in which M can represent a mixture of various ions M^{z+}, for example Fe and Ti, in which, for each metal ion, z may be identical or different, for example 2, 3 or 4 or a mixture of 2, 3 and 4.

In one particular embodiment, M^{z+} represents octahedral trivalent Fe with z equal to 3. In this embodiment, the Fe has a coordination number of 6.

The term “coordination number” is intended to mean the number of bonds for which the two electrons shared in the bond originate from the same atom. The electron-donating atom acquires a positive charge, while the electron-accepting atom acquires a negative charge.

The metal ions may be isolated or grouped into metal “clusters”. The MOF solids according to the invention may, for example, be constructed from octahedral chains or from octahedral trimers.

For the purpose of the present invention, the term “metal cluster” is intended to mean a group of atoms containing at least two metal ions linked via ionocovalent bonds, either directly via anions, for example O, OH, Cl, etc., or via the organic ligand.

Furthermore, the MOF solids according to the invention may be in various forms or “phases”, given the various possibilities for organization and connection of the ligands to the metal ion or to the metal group.

For the purpose of the present invention, the term “phase” is intended to mean a hybrid composition comprising at least one metal and at least one organic ligand having a defined crystal structure.

The crystalline spatial organization of the solids of the present invention is the basis of the particular characteristics and properties of these materials. It governs in particular the pore size, which has an influence on the specific surface area of the materials and on the adsorption characteristics. It also governs the density of the materials, said density being relatively low, the proportion of metal in these materials, the stability of the materials, the rigidity and flexibility of the structures, etc.

In addition, the pore size may be adjusted through the choice of appropriate ligands L.

In particular, the ligand L of the unit of formula (I) of the present invention may be a di-, tri-, tetra- or hexacarboxylate ligand chosen from the group comprising: C₂H₂ (CO₂⁻)₂ (fumarate), C₂H₄ (CO₂⁻)₂ (succinate), C₃H₆ (CO₂⁻)₂ (glutarate), C₄H₄ (CO₂⁻)₂ (muconate), C₄H₈(CO₂⁻)₂ (adipate), C₅H₃S(CO₂⁻)₂ (2,5-thiophenedicarboxylate), C₆H₄(CO₂⁻)₂ (terephthalate), C₆H₂N₂ (CO₂⁻)₂ (2,5-pyrazinedicarboxylate) C₁₀H₆ (CO₂⁻)₂ (naphthalene-2,6-dicarboxylate), C₁₂H₈(CO₂⁻)₂ (biphenyl-4,4′-dicarboxylate), C₁₂H₈N₂ (CO₂⁻)₂ (azobenzenedicarboxylate), C₁₂H₆Cl₂N₂(CO₂⁻)₂ (dichloroazobenzenedicarboxylate), C₁₂H₆N₂ (CO₂⁻)₂ (azobenzenetetracarboxylate), C₁₂H₆N₂(OH)₂(CO₂⁻)₂ (dihydroxoazobenzenedicarboxylate), C₆H₃ (CO₂⁻)₃ (benzene-1,2,4-tricarboxylate), C₆H₃(CO₂⁻)₃ (benzene-1,3,5-tricarboxylate), C₂₄H₁₅(CO₂⁻)₃ (benzene-1,3,5-tribenzoate) C₄₂H₂₇ (CO₂⁻) ₃ (1,3,5-tris[4′-carboxy(1,1′-biphenyl-4-yl)]benzene), C₆H₂(CO₂⁻)₃ (benzene-1,2,4,5-tetracarboxylate), C₁₀H₄(CO₂⁻)₄ (naphthalene-2,3,6,7-tetracarboxylate), C₁₀H₄(CO₂⁻)₄ (naphthalene-1,4,5,8-tetracarboxylate), C₁₂H₆(CO₂⁻)₄ (biphenyl-3,5,3′,5′-tetracarboxylate), and modified analogs chosen from the group comprising 2-aminoterephthalate, 2-nitroterephthalate, 2-methylterephthalate, 2-chloroterephthalate, 2-bromoterephthalate, 2,5-dihydroxoterephthalate, tetrafluoroterephthalate, 2,5-dicarboxyterephthalate, dimethyl-4,4′-biphenyldicarboxylate, tetramethyl-4,4′-biphenyldicarboxylate and dicarboxy-4,4′biphenyldicarboxylate.

In particular, the anion X of the unit of formula (I) of the present invention can be chosen from the group comprising OH⁻, Cl⁻, F⁻, R═(COO)ₙ⁻, PF₆⁻ and ClO₄⁻, with R and n as defined above.

In particular, the MOF solid according to the invention may comprise a mass percentage of M in the dry phase of from 5% to 50%, preferably from 18% to 31%.

The mass percentage (m %) is a unit of measurement used in chemistry and metallurgy for denoting the composition of a mixture or of an alloy, i.e. the proportions of each component in the mixture.

1 m % of a component=1 g of the component per 100 g of mixture, or alternatively 1 kg of said component per 100 kg of mixture.

The MOF solids of the present invention have in particular the advantage of being heat-stable up to a temperature of 350° C. More particularly, these solids have heat stability from 120° C. to 350° C.

In particular, the MOF solid according to the invention can have a pore size of from 0.4 to 6 nm, preferably from 0.5 to 5.2 nm, and more preferably from 0.5 to 3.4 nm.

In particular, the MOF solid according to the invention can have a specific surface area (BET) of from 5 to 6000 m²/g, preferably from 5 to 4500 m²/g.

In particular, the MOF solid according to the invention can have a pore volume of from 0 to 4 cm³/g, preferably from 0.05 to 2 cm³/g.

In the context of the invention, the pore volume means the volume accessible to the gas molecules.

The MOF solid of the invention can have a gas loading capacity of from 0.5 to 50 mmol of gas per gram of dry solid.

In the context of the present invention, the loading capacity means the capacity for storing gas or the amount of gas adsorbed into the material. The loading capacity can be expressed as mass capacity (gram/gram) or as molar capacity (mol/mol) or in other terms (mol/gram, gram/mol, etc.).

As indicated above, in the MOF solids of the invention, at least a part of the Lewis base gas(es) coordinates with M. Advantageously, at least 1 to 5 mmol of gas per gram of dry solid coordinates with M.

The part of the gas(es) which does not coordinate with M can advantageously fill the free space in the pores.

The MOF solid of the present invention may be in the form of a robust structure, which has a rigid framework and contracts only very little when the pores empty out their content, which may be, for example, solvent, noncoordinated carboxylic acid, etc. It may also be in the form of a flexible structure, which may swell and shrink, causing the aperture of the pores to vary as a function of the nature of the adsorbed molecules, which may be, for example, solvents and/or gases.

For the purpose of the present invention, the term “rigid structure” is intended to mean structures that swell or contract very sparingly, i.e. with an amplitude of up to 10%.

In particular, the MOF solid according to the invention may have a rigid structure that swells or contracts with an amplitude ranging from 0 to 10%.

The rigid structures may, for example, be constructed on the basis of octahedral trimers or chains.

According to one embodiment of the invention, the MOF solid of rigid structure may have a mass percentage of M in the dry phase of from 5% to 50%, preferably from 18% to 31%. Advantageously, M will here represent iron.

The MOF solid of rigid structure according to the invention may have a pore size of from 0.4 to 6 nm, in particular from 0.5 to 5.2 nm, more particularly from 0.5 to 3.4 nm.

The MOF solid of rigid structure according to the invention may have a pore volume of from 0.5 to 4 cm³/g, in particular from 0.05 to 2 cm³/g.

For the purpose of the present invention, the term “flexible structure” is intended to mean structures that swell or contract with large amplitude, in particular with an amplitude of greater than 10%, preferably greater than 50%.

The flexible structures may, for example, be constructed on the basis of octahedral trimers or chains.

In particular, the MOF material according to the invention may have a flexible structure that swells or contracts with an amplitude of from 10% to 300%, preferably from 50% to 300%.

In one particular embodiment of the invention, the MOF solid of flexible structure may have a mass percentage of M in the dry phase of from 5% to 40%, preferably from 18% to 31%. Advantageously, M will here represent iron.

For example, in the context of the invention, the MOF solid of flexible structure may have a pore size of from 0.4 to 6 nm, in particular from 0.5 to 5.2 nm, and more particularly from 0.5 to 1.6 nm.

For example, the MOF solid of flexible structure according to the invention may have a pore volume of from 0 to 3 cm³/g, in particular from 0 to 2 cm³/g.

In addition, the inventors have demonstrated experimentally that the amplitude of the flexibility depends on the nature of the ligand and of the solvent used, as described in the “Examples” section below.

Various MOF materials have been developed by the inventors at the Institut Lavoisier of Versailles, known as “MIL” (for “Matériau Institut Lavoisier” [Institut Lavoisier Material]). The name “MIL” for these structures is followed by an arbitrary number n given by the inventors in order to identify the various solids.

In the context of the present invention, the inventors have demonstrated that MOF solids can comprise a three-dimensional succession of units corresponding to formula (I).

In one embodiment of the invention, the MOF solids can comprise a three-dimensional succession of iron(III) carboxylates corresponding to formula (I). These iron(III) carboxylates can be chosen from the group comprising MIL-88, MIL-89, MIL-96, MIL-100, MIL-101 and MIL-102, and more particularly from the group comprising MIL-88A, MIL-88B, MIL-88Bt, MIL-88C, MIL-88D, MIL-88E, MIL-89, MIL-96, MIL-100, MIL-101 and MIL-102. These units are presented in the “Examples” section.

In particular, the MOF solids can comprise a three-dimensional succession of units corresponding to formula (I), which are chosen from the group comprising:
Fe₃OX[C₂H₂(CO₂)₂]₃ of flexible structure, for example MIL-88A,
Fe₃OX[C₆H₄(CO₂)₂]₃ of flexible structure, for example MIL-88B,
Fe₃OX[C₁₀H₆(CO₂)₂]₃ of flexible structure, for example MIL-88C,
Fe₃OX[C₁₂H₈(CO₂)₂]₃ of flexible structure, for example MIL-88D,
Fe₃OX[C₄H₄(CO₂)₂]₃ of flexible structure, for example MIL-89,
Fe₁₂O(OH)₁₈ (H₂O)₃[C₆H₃ (CO₂)₃]₆ of rigid structure, for example MIL-96,
Fe₃OX[C₆H₃(CO₂)₃]₂ of rigid structure, for example MIL-100,
Fe₃OX[O₂C—C₆H₄—CO₂]₃ of rigid structure, for example MIL-101,
Fe₆O₂X₂[C₁₀H₂(CO₂)₄]₃ of rigid structure, for example MIL-102,
   in which X is as defined above.

In addition, from the same carboxylic acid ligand L and the same iron bases (trimers), the inventors have been able to obtain MOF materials of the same general formula (I) but having different structures. This is, for example, the case for the solids MIL-88B and MIL-101. Specifically, the difference between the solids MIL-88B and MIL-101 lies in the mode of connection of the ligands to the octahedral trimers: in the MIL-101 solid, the ligands L assemble in the form of rigid tetrahedra, whereas in the MIL-88B solid, they form trigonal bipyramids, enabling spacing between the trimers.

These various materials are presented in the “Examples” section below. The mode of assembly of these ligands can be controlled during the synthesis, for example by adjusting the pH. For example, the MIL-88 solid is obtained in a less acidic medium than the MIL-101 solid, as described in the “Examples” section below.

In addition, the MOF solids of the invention can make it possible to graft molecules onto their surface so as to satisfy the needs associated with the vectorization of compounds toward specific biological targets and/or with the stealth of the particles. This thus makes it possible to improve the biodistribution of the active ingredients in a more targeted manner.

Thus, according to one particular embodiment, the MOF solids according to the invention can optionally comprise on their surface at least one organic surface agent. This agent may be grafted or deposited on the surface of the solids, for example adsorbed onto the surface or bonded via covalent bonding, for example hydrogen bonding, via Van der Waals bonding or via electrostatic interaction. The surface agent may also be incorporated by entanglement during the manufacture of the MOF solids [10, 28].

According to the invention, the term “surface agent” is intended to mean a molecule that partly or totally covers the surface of the solid, making it possible to modulate the surface properties of the material, for example:
to modify its biodistribution, for example so as to avoid its recognition by the reticuloendothelial system (“stealth”), and/or
to give it advantageous bioadhesion properties during oral, ocular or nasal administration, and/or
to enable it to perform specific targeting of certain diseased organs/tissues, etc.

According to the invention, several surface agents may be used in order to combine the above-mentioned properties.

According to the invention, the organic surface agent may be chosen, for example, from the group comprising:
an oligosaccharide, for instance cyclodextrins,
a polysaccharide, for instance chitosan, dextran, fucoidan, alginate, pectin, amylose, starch, cellulose or xylan,
a glycosaminoglycan, for instance hyaluronic acid or heparin,
a polymer, for instance polyethylene glycol (PEG), polyvinyl alcohol or polyethyleneimine,
a surfactant, for instance pluronic or lecithin,
vitamins, for instance biotin,
coenzymes, for instance lipoic acid,
antibodies or antibody fragments,
amino acids or peptides.

The surface agent may also be a targeting molecule, i.e. a molecule which recognizes or is specifically recognized by a biological target. The combination of the MOF solids of the invention with a targeting molecule thus makes it possible to vectorize the products toward this biological cell, tissue or organ target.

Thus, the organic surface agent may be a targeting molecule chosen from the group comprising biotin, chitosan, lipoic acid, an antibody or antibody fragment, and a peptide.

For example, the presence of biotin at the surface can be exploited in order to easily couple ligands, for example by simple incubation. To do this, it is possible to use protocols described in the publications [29, 30].

This surface-modification method has the advantage of not disturbing the core of the MOF solids, even when they contain gas, and of being able to be carried out after the synthesis of the MOF solids, and thus of offering a variety of possible coverings.

It is also possible to use a blend of polymers bearing functions capable of interacting with the particle (MOF) as surface agent in order to satisfy precise specifications, for example bioadhesion, specific recognition, etc.

The subject of the invention is also a method for preparing MOF solids as defined in the present invention, comprising at least one reaction step consisting:
(i) in mixing in a polar solvent:
   at least one solution comprising at least one metal inorganic precursor in the form of a metal M, of a metal salt of M or of a coordination complex comprising a metal ion of M,
   at least one ligand L′ comprising a radical R comprising q groups *—C(═O)—R³, in which
      q and R are as defined above,
      * denotes the point of attachment of the group with the radical R,
      R³ is chosen from the group comprising an OH, an OY, with Y being an alkali metal cation, a halogen, or a radical —OR⁴, —O—C(═O)R⁴ or —NR⁴R^{4′}, in which R⁴ and R^{4′} are C₁₋₁₂ alkyl radicals,
         so as to obtain an MOF material;
(ii) in activating the MOF material obtained in (i); and
(iii) in bringing the MOF material obtained in step (ii) into contact with a Lewis base gas, at least a part of which coordinates with M, so as to obtain said solid.

M is an ion of a transition metal as defined above.

The preparation of MOF materials may be preferably carried out in the presence of energy, which may be supplied, for example, by heating, for instance hydrothermal or solvothermal conditions, but also by microwave, by ultrasound, by grinding, by a process involving a supercritical fluid, etc. The corresponding protocols are those known to a person skilled in the art. Nonlimiting examples of protocols that can be used for the hydrothermal or solvothermal conditions are described, for example, in [20]. For the synthesis via microwaves, nonlimiting examples of protocols that can be used are described, for example, in [21, 22, 23, 24]. For the conditions in the presence of a roll mill, reference may be made, for example, to the publications [25, 26, 27].

The hydrothermal or solvothermal conditions, the reaction temperatures of which may range between 0 and 220° C., are generally performed in glass (or plastic) containers when the temperature is below the boiling point of the solvent. When the temperature is higher or when the reaction is carried out in the presence of fluorine, Teflon bodies inserted into metal bombs are used [20].

The solvents used are generally polar. In particular, the following solvents can be used: water, alcohols, dimethylformamide, dimethyl sulfoxide, acetonitrile, tetrahydrofuran, diethylformamide, chloroform, cyclohexane, acetone, cyanobenzene, dichloromethane, nitrobenzene, ethylene glycol, dimethylacetamide, or mixtures of these solvents.

One of more cosolvents may also be added at any step of the synthesis for better solubilization of the compounds of the mixture. They may in particular be monocarboxylic acids, such as acetic acid, formic acid, benzoic acid, etc.

One or more additives may also be added during the synthesis in order to modulate the pH of the mixture. These additives are chosen from inorganic or organic acids or inorganic or organic bases. By way of example, the additive may be chosen from the group comprising: HF, HCl, HNO₃, N₂SO₄, NaOH, KOH, lutidine, ethylamine, methylamine, ammonia, urea, EDTA, tripropylamine and pyridine.

Preferably, the reaction step (i) may be carried out according to at least one of the following reaction conditions:
with a reaction temperature of from 0° C. to 220° C., preferably from 50 to 150° C.;
with a stirring speed of from 0 to 1000 rpm (or revolutions per minute), preferably from 0 to 500 rpm;
with a reaction time of from 1 minute to 144 hours, preferably from 1 minute to 15 hours;
with a pH of from 0 to 7, preferably from 1 to 5;
with the addition of at least one cosolvent to the solvent, to the precursor, to the ligand or to the mixture thereof, said cosolvent being chosen from the group comprising acetic acid, formic acid and benzoic acid; in the presence of a solvent chosen from the group comprising water, R^{s}—OH alcohols in which R^{s} is a linear or branched Cᵢ to C₆ alkyl radical, dimethylformamide, dimethyl sulfoxide, acetonitrile, tetrahydrofuran, diethylformamide, chloroform, cyclohexane, acetone, cyanobenzene, dichloromethane, nitrobenzene, ethylene glycol, dimethylacetamide, or mixtures of these solvents, which may be miscible or immiscible;
in a supercritical medium, for example in supercritical CO₂;
under microwaves and/or under ultrasound;
under electrochemical electrolysis conditions;
under conditions using a roll mill;
in a gas stream.

As indicated, prior to bringing the MOF material into contact with the Lewis base gas in step (iii), it is necessary for the material derived from step (i) to be activated in step (ii).

This activation step (ii) makes it possible to empty the pores of the MOF material and to make them accessible for the coordination of the gas(es). The emptying can take place, for example, through the departure of the water molecules and/or the solvents present in the reaction medium, either by activation under a primary or secondary vacuum or under a gas stream (helium, nitrogen, air, etc.), with or without heating of the solid at a temperature of from 25 to 300° C., in particular from 50 to 250° C., and more particularly from 100 to 250° C. The heating can be carried out for a period of time of between 1 hour and 96 hours, typically between 3 and 5 hours.

Step (ii) can also be a step of reduction of the metal centers M of said MOF material to give ions in which z is from 2 to 4. According to the activation conditions, the metal centers may be partially or even totally reduced.

The metal centers may be made up of identical or different metals, for example only iron, or a mixture of one or more metals such as iron in the presence of titanium, of manganese or of zirconium.

When the metal centers are partly reduced, in particular if a part of the iron is in the oxidation state +II (z=2) or a part of the manganese is in the oxidation state +III (z=3), part of the gas molecules can then coordinate more strongly with the metal through a back-donation effect. The term “back-donation” is intended to mean the transfer of the electron density of the metal M to an antibonding orbital of the gas. This results in an increase in the number of gas molecules coordinated per metal center. The final MOF solid will then comprise a greater amount of gas molecules coordinated with the reduced metal ions. The resulting MOF solids will then have a greater gas storage capacity.

Moreover, the partial reduction of the metal centers has an influence on the duration of release of the gases by the MOF solid, said duration sizably increasing.

The activation step (ii) may also be carried out at a high temperature and under reduced pressure. The term “reduced pressure” is intended to mean a pressure ranging from 1 to 10⁻² Pa, advantageously from 10⁻³ to 10⁻⁵ Pa.

For example, the activation can be carried out at 50-250° C. under a pressure of from 1 to 10⁻² Pa, or from 10⁻³ to 10⁻⁵ Pa.

In step (iii) of the method of the invention, the MOF material activated in step (ii) is brought into contact with at least one Lewis base gas. The gas may be in pure form or as a mixture with an inert gas.

The bringing of the solid into contact with the gas in step (iii) can be carried out at a temperature ranging from −150 to 100° C.

Step (iii) can also be carried out at a pressure ranging from 10⁴ to 10⁷ Pa.

In one particular embodiment, the Lewis base gas is preferably NO. Step (iii) can then be carried out at a temperature ranging from −100° C. to +50° C. The bringing of the NO into contact with the solid can be carried out at a pressure ranging from 10⁶ to 10⁶ Pa.

Depending on the application envisioned, a mixture of Lewis base gases can be used in step (iii) of the method.

The MOF solid according to the invention can have a gas loading capacity of from 0.5 to 50 mmol of gas per gram of dry solid.

As indicated above, at least a part of the gas can coordinate with M. Advantageously, the solid according to the invention can have at least 1 to 5 mmol of gas per gram of dry solid coordinated with M.

According to one particular embodiment of the invention, when the MOF solids of the invention comprise at least one surface agent at their surface, the method for preparing the MOF solids according to the invention can also comprise a step (iv) of attaching at least one organic surface agent to said solid.

This attachment step (iv) can be carried out during or after the reaction step (i) or else after the activation step (ii) and before the step (iii) of bringing the MOF material into contact with the gas. Examples are provided below.

The method of preparation of the invention has the advantage of making it possible to obtain crystalline MOF solids which are pure and homogeneous, in a small number of steps and with high yields. This reduces the synthesis time and the manufacturing costs.

Moreover, the inventors have also demonstrated that the particular structural characteristics of the solids of the present invention, in particular in terms of flexibility or of pore size, make them adsorbents of high loading capacity, of high selectivity and of high purity. They therefore enable the selective adsorption of molecules of Lewis base gas of biological interest, for instance molecules of NO, CO or H₂S, with a favorable energy cost and a longer release time. Thus, the research studies carried out by the inventors have enabled them to demonstrate the advantage of the MOF materials according to the invention for the adsorption and the controlled release, controlled in terms of amount of gas and of duration of release, of gases of biological interest.

By virtue of their structure, the MOF solids of the invention make it possible to control the duration of release of the gases. Thus, with the MOF solids of the invention, the duration of release of the gases can range from 1 to 100 hours, whereas with the zeolites of the prior art, the duration of this release does not exceed 10 hours under a water vapor pressure [10, 11].

Moreover, the MOF solids of the invention have a capacity for adsorption and storage of Lewis base gases which is substantially greater than that of the known zeolites. For example, the MOFs of the invention can have an NO adsorption capacity ranging from 2.5 to 4.5 mmol/g, whereas, with the known zeolites, this capacity is less than 1.5 mmol/g. This makes it possible to reduce the amount of material to be used for a given application.

Furthermore, this greater adsorption capacity makes it possible to have materials in which the release of the gases can take place continuously. The release of the gases can also be targeted when the materials comprise, at their surface, at least one targeting agent as defined above.

The invention also relates to the use of MOF solids according to the invention, loaded with at least one Lewis base gas of biological interest, at least a part of which coordinates with M, as a medicament. Said gas or the mixture of gases can be contained in the pores and at least partly coordinated with M according to the invention.

Specifically, the MOF solids according to the invention have the advantage of having high adsorption capacities. In addition, they make it possible to efficiently adsorb molecules of gases which may exhibit particular difficulties, for example owing to their instability, their high reactivity, their low solubility, etc.

In addition, the Lewis base gas may be any electron-donating gas which is of biological interest, for instance NO, CO or H₂S [10, 28].

A particularly advantageous use of the MOFs according to the invention is in the prevention of thromboses, in particular after the insertion of stents, catheters, prosthetic conduits and other medical implants in the body following a surgical procedure. In this type of use, the MOF solids of the invention can serve, for example, as a coating for the abovementioned medical articles. As a coating, they can be used alone or in combination with other therapeutic agents. The MOFs which are loaded with gas are capable of releasing the gas (or the mixture of gases). They can then constitute an effective means for preventing the formation of thromboses in contact with the foreign body. The MOF solids of the invention can therefore be used as an antithrombotic medicament. It is also possible to envision using them alone or in combination with other known antithrombotic agents, for instance clopidogrel.

The invention also extends to the medical articles, for instance stents, catheters, prosthetic conduits and other medical implants in the body following a surgical procedure, comprising an MOF solid according to the invention. Keefer L. K., Nat, Mater. 2003, volume 2, 357 [31].

In addition, the MOF solid according to the invention can be loaded with at least one Lewis base gas of biological interest for use in the cosmetics or dermatology field. The antibacterial activity of NO may, for example, be very advantageous for applications in particular in the field of cosmetic creams. Depending on the water content of the emulsion in which the particles of MOF solids of the invention may be dispersed, a modulatable release of NO will make it possible to disinfect the skin in a sustained manner. Furthermore, the inorganic-organic nature of the MOF solids will facilitate their dispersion in the cosmetic creams.

The beneficial cosmetic or dermatological effects that may result from the use of an NO-loaded MOF solid according to the invention are, for example:
the antiwrinkle effects, the reconstitution of the fullness of the lips and the heightening of their natural red color, the stimulation of the natural pink color of the skin and the homogenizing of the complexion, through the action of vasodilation and/or increased blood circulation in the skin;
the reduction of skin damage caused by UV light;
the antibacterial effect in the treatment of acne.

As already indicated, the MOF solids of the invention have the advantage of having unexpected loading capacities, as yet never achieved in the prior art materials.

They also have the advantage of enabling release times that can be modulated by virtue of their structure. Specifically, the rigid or flexible nature of the structures of the MOF solids of the invention has an influence on the gas release kinetics and the content of gas released. The MOF solids with a flexible structure can in particular enable release with a modulatable duration. Thus, MOF solids of flexible structure comprising hydrophobic ligands will make it possible to modulate the duration of the release of the gas.

The use of the MOFs of flexible structures as defined above can thus constitute a credible alternative for release of a gas of biological interest, such as NO, over a longer period of time.

When loaded with a suitable gas (or mixture of gases), these solids, for which the first toxicity tests show that they are not, a priori, toxic, may also be of great benefit in the food industry, in particular as an antibacterial agent, for example for preserving foods and industrial preparations. In particular, when food preparations are placed under a vacuum, the introduction of a small amount of MOF solid according to the invention, combined with the presence of water naturally present in the foods, can result in a slow and continuous release of NO which makes it possible to inhibit bacterial growth and to destroy the microorganisms.

Another subject of the invention is a pharmaceutical, cosmetic or dermatological composition comprising an MOF solid according to the invention and a pharmaceutically or cosmetically acceptable vehicle.

In addition, the MOF solids according to the invention have been the subject of very positive toxicity studies, described in the “Examples” section. They also appear to be biodegradable and the degradability studies are still ongoing.

Thus, the MOF solids of the present invention, used for the release of a gas of biological interest, make it possible to overcome the previously mentioned problems associated with toxicity in the prior art.

This is because the use of MOFs based on metals of low toxicity makes it possible to envision applications under biological conditions.

In one particular embodiment of the invention, the MOF solids may be loaded both with gas and with a pharmaceutically active ingredient. The pharmaceutically active ingredient can be contained in the pores. This makes it possible to obtain a combined therapeutic effect.

In particular, the MOF solids according to the invention can be loaded with pharmaceutically active ingredient, with a loading capacity of from 1% to 200% by weight of dry solid, for example from 1% to 70% by weight of dry solid, that is to say close to from 10 to 700 mg per gram of dry solid.

Thus, the invention extends to the use of such solids as a medicament.

When the MOF solids of the invention are loaded both with gas and with a pharmaceutically active ingredient, the method for preparing them may also comprise a step (v) of introducing, into said solid, at least one molecule of interest, which may be a pharmaceutically active ingredient.

Said introduction step can be carried out during reaction step (i) or after said step, so as to obtain a solid loaded with molecule of interest.

Any method known to a person skilled in the art can be used during introduction step (v). The molecule of interest can be, for example, introduced into the MOF material of the present invention:
by impregnation, by immersing the material in a solution of the molecule of interest;
by sublimation of the molecule of interest, and then the gas is adsorbed by the material; or

In another embodiment, the MOF solids of the invention, loaded with a gas or with a mixture of gases, comprises a ligand L which itself has a therapeutic activity. A combined therapeutic effect can then be obtained by means of the release of the gas(es) and the activity of L after dissolution of the material.

Other advantages may become apparent to a person skilled in the art on reading the examples below, illustrated by the attached figures, given by way of illustration.

### BRIEF DESCRIPTION OF THE FIGURES

FIG. 1 represents an X-ray diffractogram of the solid MIL-100(Fe).

FIG. 2 represents a nitrogen adsorption isotherm at 77 K of the solid MIL-100 (Po=1 atm).

FIG. 3 represents a thermogravimetric analysis (in air, with a heating rate of 2° C./minute) of the compound MIL-100(Fe).

FIG. 4 represents the X-ray diffractogram of the solid MIL-101(Fe) (λ_{Cu}=1.5406 Å).

FIG. 5 represents a thermogravimetric analysis (in air, with a heating rate of 2° C./minute) of the compound MIL-101(Fe).

FIG. 6 represents X-ray diffractograms of the solids MIL-88A crude (top curve) and suspended in water (bottom curve).

FIG. 7 represents a thermogravimetric analysis (in air, with a heating rate of 2° C./minute) of the hydrated compound MIL-88A(Fe).

FIG. 8 represents X-ray diffractograms of the solids MIL-88B dry (bottom curve (b)) and hydrated (top curve (a)).

FIG. 9 represents a thermogravimetric analysis (in air, with a heating rate of 2° C./minute) of the hydrated compound MIL-88B.

FIG. 10 represents X-ray diffractograms of the solids MIL-89 dry (curve a), DMF (curve b) and hydrated (curve c).

FIG. 11 represents an X-ray diffractogram of the solid MIL-88C.

FIG. 12 represents a thermogravimetric analysis (in air, with a heating rate of 2° C./minute) of the crude synthetic compound MIL-88C.

FIG. 13 represents X-ray diffractograms of the solids MIL-88D crude (bottom curve (b)) and hydrated (top curve (a)).

FIG. 14 represents a thermogravimetric analysis (in air, with a heating rate of 2° C./minute) of the hydrated compound MIL-88D(Fe).

FIG. 15 represents X-ray diffractograms of the solid MIL-88B-NO₂ crude (top curve (a)) and hydrated (bottom curve (b)).

FIG. 16 represents a thermogravimetric analysis (in air, with a heating rate of 2° C./minute) of the compound MIL-88B-NO₂(Fe) after washing and drying.

FIG. 17 represents X-ray diffractograms of the solid MIL-88B-2OH crude (bottom curve (c)), hydrated (middle curve (b)) and dried under vacuum (top curve (a)).

FIG. 18 represents a thermogravimetric analysis (in air, with a heating rate of 2° C./minute) of the hydrated compound MIL-88B-2OH(Fe).

FIG. 19 represents X-ray diffractograms of the crude solid MIL-88B-NH₂ (bottom curve (b)) and of the dry solid MIL-88B-NH₂ (top curve (a)).

FIG. 20 represents a thermogravimetric analysis (in air, with a heating rate of 2° C./minute) of the hydrated compound MIL-88B-NH₂(Fe).

FIG. 21 represents X-ray diffractograms of the solids MIL-88B-CH₃ crude (top curve (a)), hydrated (middle curve (b)) and solvated with DMF (bottom curve (c)).

FIG. 22 represents X-ray diffractograms of the solid MIL-88B-Cl crude (bottom curve (b)) and hydrated (top curve (a)).

FIG. 23 represents a thermogravimetric analysis (in air, with a heating rate of 2° C./minute) of the hydrated compound MIL-88B-Cl(Fe).

FIG. 24 represents X-ray diffractograms of the solid MIL-88B-4-CH₃ crude (bottom curve (b)) and hydrated (top curve (a)).

FIG. 25 represents a thermogravimetric analysis (in air, with a heating rate of 2° C./minute) of the hydrated compound MIL-88B-4-CH₃(Fe).

FIG. 26 represents X-ray diffractograms of the solids MIL-88B-4F crude (bottom curve (c)), hydrated (curve (b)) and solvated with EtOH (top curve (a)).

FIG. 27 represents a thermogravimetric analysis (in air, with a heating rate of 2° C./minute) of the hydrated compound MIL-88B-4F(Fe).

FIG. 28 represents X-ray diffractograms of the solids MIL-88B-Br crude (bottom curve (b)) and hydrated (top curve (a)).

FIG. 29 represents a thermogravimetric analysis (in air, with a heating rate of 2° C./minute) of the hydrated compound MIL-88B-Br(Fe).

FIG. 30 represents an X-ray diffractogram of the solid MIL-88E(Fe).

FIG. 31 represents X-ray diffractograms of the solids MIL-88F crude (bottom curve (b)) and hydrated (top curve (a)).

FIG. 32 represents a thermogravimetric analysis (in air, with a heating rate of 2° C./minute) of the hydrated compound MIL-88F(Fe).

FIG. 33 represents X-ray diffractograms of the solids MIL-88D-4-CH₃ crude (bottom curve (b)) and hydrated (top curve (a)).

FIG. 34 represents a thermogravimetric analysis (in air, with a heating rate of 2° C./minute) of the hydrated compound MIL-88D-4-CH₃(Fe).

FIG. 35 represents X-ray diffractograms of the solids MIL-88D-2CH₃ crude (bottom curve (c)), hydrated (curve (b)) and wetted (top curve (a)).

FIG. 36 represents a thermogravimetric analysis (in air, with a heating rate of 2° C./minute) of the crude synthetic compound MIL-88D-2CH₃(Fe).

FIG. 37 represents an X-ray diffractogram of the solid MIL-88G crude (bottom curve (c)), solvated with DMF (middle curve (b)) and solvated with pyridine (top curve (a)).

FIG. 38 represents a thermogravimetric analysis (in air, with a heating rate of 2° C./minute) of the crude synthetic compound MIL-88G(Fe).

FIG. 39 represents an X-ray diffractogram of the solid MIL-88G-2Cl crude (bottom curve (b)) and dry (top curve (a)).

FIG. 40 represents a thermogravimetric analysis (in air, with a heating rate of 2° C./minute) of the crude synthetic compound MIL-88G-2Cl (Fe).

FIG. 41 represents X-ray diffractograms of the solids MIL-102(Fe) crude (curve (a)) and reference MIL-102 (Cr) (curve (b)).

FIG. 42 represents a thermogravimetric analysis (in air) of the crude synthetic compound MIL-102(Fe).

FIG. 43 represents a reaction scheme for obtaining 3,5,3′,5′-tetramethylbiphenyl-4,4′-dicarboxylic acid.

FIG. 44 represents a reaction scheme for obtaining 3,3′-dimethylbiphenyl-4,4′-dicarboxylic acid.

FIG. 45 represents an image obtained by SEM (Scanning Electron Microscopy) of the solid MIL-89 nano.

FIG. 46 represents an image obtained by SEM (Scanning Electron Microscopy) of the solid MIL-88Anano.

FIG. 47 represents an image obtained by SEM (Scanning Electron Microscopy) of the solid MIL-100 nano.

FIG. 48 represents an image obtained by SEM of the solid MIL-88Btnano.

FIG. 49 represents an image obtained by SEM of the solid MIL-88Bnano.

FIG. 50 represents an amount of unsaturated iron sites present in MIL-100 Fe activated under vacuum at various temperatures.

FIG. 51 represents an NO adsorption isotherm et 298 K for the iron carboxylate MIL-100(Fe) activated at 120° C. overnight.

FIG. 52 represents a profile for release of NO (NOᵣₑₗ in mmol/g) under a vapor pressure from the solid MIL-100(Fe) as a function of the time t (in hours).

FIG. 53 represents (on the left): an NO adsorption isotherm at 298 K for MIL-100(Fe) activated at 250° C. under vacuum overnight; (on the right): a profile for release of NO under a vapor pressure from the solid MIL-100(Fe) activated at 250° C. under vacuum overnight.

FIG. 54 represents a schematic view of the phenomenon of respiration (swelling and contraction) in the solids MIL-88A, MIL-88B, MIL-88C, MIL-88D and MIL-89. The swelling amplitude between the dry forms (at the top) and open forms (at the bottom) is represented as a percentage at the bottom of the figure.

FIG. 55 represents NO adsorption isotherms at 298 K for the iron carboxylates MIL-88A(Fe) and MIL-88B(Fe) activated at 150° C. overnight.

FIG. 56 represents profiles for release of NO under a water vapor pressure from the solids MIL-88A(Fe) (at the top) and MIL-88B(Fe) (at the bottom) activated at 150° C. overnight.

FIG. 57 represents, at the top, a study of the reversibility of swelling of the solid MIL-88A by X-ray diffraction (λ˜1.79 Å), and, at the bottom, X-ray diffractograms of the solid MIL-88A in the presence of solvents (λ˜1.5406 Å).

FIG. 58 represents an explanatory scheme of the flexibility in the hybrid phases MIL-53 (a) and MIL-88 (b and c).

FIG. 59 represents the crystallographic structure of MIL-126(Fe). The FeO₆ polyhedra are represented with or without a star, the two MIL-88D frameworks being indicated. The carbon atoms are in black.

FIG. 60 represents an X-ray diffractogram of the solid MIL-126(Fe) (λ_{Cu}=1.5406 Å).

FIG. 61 represents a thermogravimetric analysis (in air, with a heating rate of 2° C./minute) of the compound MIL-126(Fe).

FIG. 62 represents nitrogen adsorption isotherms for MIL-126(Fe) (P₀=1 atmosphere).

FIG. 63 represents an X-ray diffractogram of iron 3,3′,5,5′-azobenzenetetracarboxylate (λ_{cu}=1.5406 Å).

FIG. 64 represents the X-ray diffractogram of the crude solid iron 2,5-dihydroxoterephthalate. The phase, of trigonal symmetry, is an isotype of that published by Dietzel et al. with cobalt and nickel (space group R3) [61].

FIG. 65 represents a thermogravimetric analysis (in air, with a heating rate of 2° C./minute) of the compound iron 3,3′,5,5′-azobenzenetetracarboxylate.

FIG. 66 represents nitrogen adsorption isotherms for iron 3,3′,5,5′-azobenzenetetracarboxylate (P₀=1 atmosphere).

FIG. 67 represents the XRD diagrams of the unmodified material MIL-88A before (MIL88A) and after the addition of one drop of water (MIL88A+H₂O); MIL-88A modified with 7% chitosan before (MIL88AQ100) and after the addition of one drop of water (MIL88A Q100+H₂O); MIL-88A modified with 2% chitosan before (MIL88AQ25) and after the addition of one drop of water (MIL88A Q25+H₂O).

FIG. 68 represents the thermogravimetric analysis of the unmodified material MIL-88A (MIL88A; green), the material ML-88A modified with 2% chitosan (MIL-88A-Q25, black) and the material MIL-88A modified with 7% chitosan (MIL-88A-Q100, red).

FIG. 69 represents the confocal microscopy images of the material MIL-100(Fe) surface-modified with dextran-fluorescein-biotin.

FIG. 70 represents the change in particle size (P in nm) for MIL-88A surface-modified with polyethylene glycol, as a function of time (t in min).

FIG. 71 represents the profiles for release of NO under water vapor pressure (curve (a)) and in phosphate buffer (curve (b)) from solid MIL-88A(Fe). The amount of NO released (NOᵣₑₗ in mmol·g⁻¹, on the left, and ppm NO, on the right) is expressed as a function of the time t (in hours).

FIG. 72 represents the profiles for release of NO under water vapor pressure (curve (a)) and in phosphate buffer (curve (b)) from solid MIL-88B(Fe). The amount of NO released (NOᵣₑₗ in mmol·g⁻¹, on the left, and ppm NO, on the right) is expressed as a function of the time t (in hours).

FIG. 73 represents the X-ray diffractogram of the solid MIL-88A-nano obtained by microwave synthesis.

FIG. 74 represents the NO adsorption isotherms at 298 K for the iron carboxylates MIL-88A(Fe)-nano activated at 150° C. under vacuum overnight. The amount of NO (NO_{abs} in mmol·g⁻¹) adsorbed (curve (a)) and desorbed (curve (b)) is represented as a function of the pressure P (in mmHg).

FIG. 75 represents the profiles for release of NO under water vapor pressure from the solids MIL-88A(Fe) (5 microns, curve (a)) and MIL-88A(Fe)-nano (120 nm, curve (b)). The amount of NO released (NOᵣₑₗ in mmol·g⁻¹, on the left, and ppm NO, on the right) is expressed as a function of the time t (in hours).

FIG. 76 represents the NO adsorption isotherms at 298 K from the iron carboxylates MIL-88B(Fe)-NO₂ activated at 150° C. under vacuum overnight. The amount of NO(NO_{abs} in mmol·g⁻¹) adsorbed (curve (a)) and desorbed (curve (b)) is represented as a function of the pressure P (in mmHg).

FIG. 77 represents the NO adsorption isotherms at 298 K for the iron carboxylates MIL-88B(Fe)-2OH activated at 80° C. under vacuum overnight. The amount of NO(NO_{abs} in mmol·g⁻¹) adsorbed (curve (a)) and desorbed (curve (b)) is represented as a function of the pressure P (in mmHg).

FIG. 78 represents the profiles for release of NO under water vapor pressure (curve (a)) and in phosphate buffer (curve (b)) from solid MIL-88B(Fe)—NO₂. The amount of NO released (NOᵣₑₗ in mmol·g⁻¹, on the left, and in ppm NO, on the right) is expressed as a function of the time t (in hours).

FIG. 79 represents the profiles for release of NO under water vapor pressure (curve (a)) and in phosphate buffer (curve (b)) from solid MIL-88B(Fe)-2OH. The amount of NO released (NOᵣₑₗ in mmol·g⁻¹, on the left, and in ppm NO, on the right) is expressed as a function of the time t (in hours).

FIG. 80 represents the profiles for release of NO under water vapor pressure from the solids MIL-100Fe (curve (a)), MIL-88A (curve (b)), MIL-88B (curve (c)), MIL-88-2OH (curve (d)) and MIL-88B-NO₂ (curve (e)). The amount of NO released (NOᵣₑₗ in mmol·g⁻¹) is expressed as a function of the time t (in hours).

FIG. 81 represents the NO adsorption isotherms at 298 K for the solid MIL-22 activated at 350° C. under vacuum overnight. The amount of NO (NO_{abs} in mmol·g⁻¹) adsorbed (curve (a)) and desorbed (curve (b)) is represented as a function of the pressure P (in mmHg).

FIG. 82 represents the profiles for release of NO by solid MIL-22 under water vapor pressure. The amount of NO released (NOᵣₑₗ in mmol·g⁻¹, on the left, and in ppm NO, on the right) is expressed as a function of the time t (in hours).

FIG. 83 represents the CO adsorption isotherm (CO_{ads} in mmol/g) for the solid MIL-100(Fe) at the temperature of 303 K as a function of the pressure P (in bar) for the iron carboxylate MIL-100(Fe) activated at 100° C. for 12 h (100° C. curve), 250° C. for 12 h (250° C. (1) curve) and 250° C. for 20 h (250° C. (2) curve).

FIG. 84 represents the histological sections of the liver observed by Proust staining (iron colored blue). They show an accumulation of iron in the liver.

FIG. 85 represents the NO adsorption isotherms at 298 K for the solids CPO-27 (Co dihydroxoterephthalate) (curves (a) and (b)), CPO-27 (Ni 2,5-dihydroxoterephthalate; M₂(dhtp)(H₂O).xH₂O (M=Ni or Co dhtp=2,5-dihydroxyterephthalic acid, x˜8)) (curves (c) and (d)), MIL-100 (Fe trimesate) (curves (e) and (f)), HKUST (Cu trimesate) (curves (g) and (h)), MIL-53 (Al terephthalate) (curves (i) and (j)) and MIL-53 (Cr terephthalate) (curves (k) and (l)).

FIG. 86 represents the profiles for release of NO under water vapor pressure from the solid CPO-27 (Co dihydroxoterephthalate) (curve (a)), CPO-27 (Ni 2,5-dihydroxoterephthalate; M₂(dhtp)(H₂O).xH₂O (M=Ni or Co dhtp=2,5-dihydroxyterephthalic acid, x˜8)) (curve (b)), HKUST-1 (Cu trimesate) (curve (c)), MIL-53 (Al terephthalate) (curve (d)) and MIL-53 (Cr terephthalate) (curve (e)). The amount of NO released (NOᵣₑₗ in mmol·g⁻¹) is expressed as a function of the time t (in hours).

FIG. 87 represents the profiles for release of NO under water vapor pressure from the solid MIL-88A (3 samples under the same conditions) in the form of a cream. The amount of NO released (NOᵣₑₗ in mmol·g⁻¹) is expressed as a function of the time t (in hours).

FIG. 88 represents the profiles for release of NO under water vapor pressure from the solid MIL-88A-nano in the form of a cream (curve (b)) and in the form of a powder (curve (a)) in comparison with the release in a PBS solution (curve (c)). The amount of NO released (NOᵣₑₗ in mmol·g⁻¹) is expressed as a function of the time t (in hours).

FIG. 89 represents an X-ray diffractogram of iron nicotinate 2: space group P 21/n: a=16.422899, b=21.423-401, c=11.048300 beta=91.806999.

### EXAMPLES

### Example 1

### Synthesis of and Data on the Iron Carboxylates of the Present Invention

This example describes the synthesis of various iron carboxylates. The solids obtained were subsequently characterized according to the methods described below.

The analysis of the crystal structure of the iron carboxylate solids was carried out by X-ray (XR) diffraction using a Siemens D5000 diffractometer (radiation CuKα λ_{Cu}=1.5406 Å, mode θ-2θ), at ambient temperature in air. The diagrams are represented either in angular distances (2θ, in degrees °) or in interreticular distances (d, in Å or Angstrom).

The characterization of the porosity (Langmuir specific surface area and pore volume) of the solids was measured by the nitrogen adsorption at 77 K with a Micromeretics ASAP-2010 instrument. The solids were dehydrated beforehand at 150° C. under a primary vacuum overnight. The isotherm for nitrogen adsorption by the solids is given by a curve representing the volume of nitrogen adsorbed V (in cm³/g) as a function of the ratio of the pressure P to the reference pressure P₀=1 atm.

The thermogravimetric analysis was carried out under an air atmosphere using a TA-instrument model 2050 instrument. The heating rate was 2° C./minute. The curve resulting from the thermogravimetric analysis of the solids represents the loss of mass Lm (as %) as a function of the temperature T (in ° C.).

The elemental analysis of the solids was carried out by the central analysis department of the CNRS [French National Center for Scientific Research] of Vernaison: Organic analysis:

Microanalyses C,H,N,O,S in the pharmaceutical products, the polymers and, in general, the synthetic products, by coulometric detection, catharometric detection or infrared cell detection.

### Inorganic Analysis:

The main techniques used:
ICP-AES (Inductive Coupled Plasma-Atomic Emission Spectroscopy) with various types of detectors,
ICP-MS (Inductively Coupled Plasma-Mass Spectrometry) with quadrupole or magnetic-sector mass spectrometers,
CVAAS (Cold-Vapor Atomic Absorption Spectroscopy),
ICP/MS/HPLC coupling (Inductively Coupled Plasma/Mass Spectrometry/High Performance Liquid Chromatography),
X-ray fluorescence,
Wet, dry or microwave treatment of samples.
   a) MIL-100(Fe) or Fe₃O[C₆H₃—(CO₂)₃]₂.X.nH₂O (X═F, Cl, OH)

The iron carboxylate MIL-100(Fe) was synthesized according to two conditions: with and without hydrofluoric acid.

Synthesis Conditions with Hydrofluoric Acid:

56 mg of iron metal powder (1 mmol, sold by the company Riedel de Haen, 99%) and 140 mg of 1,3,5-benzenetricarboxylic acid (0.6 mmol, 1,3,5-BTC; sold by the company Aldrich, 99%) are dispersed in 5 ml of distilled water with 0.6 ml of 2M nitric acid (sold by the company VWR, 50%) and 0.4 ml of 5M hydrofluoric acid (sold by the company SDS, 50%). The whole mixture is placed in a 23 ml Teflon body placed in a metal bomb from the company Paar and left for 6 days at 150° C. with a temperature rise stage of 12 hours and a temperature drop stage of 24 hours. The solid is recovered by filtration.

The solid (200 mg) is then suspended in 100 ml of distilled water at reflux with stirring for 3 h in order to remove the trimesic acid remaining in the pores. The solid is then recovered by hot filtration.

Synthesis Conditions without Hydrofluoric Acid:

0.27 g of FeCl₃.6H₂O (1 mmol, sold by the company Alfa Aesar, 98%) and 140 mg (0.6 mmol) of 1,3,5-benzenetricarboxylic acid (1,3,5-BTC; sold by the company Aldrich, 99%) are dispersed in 5 ml of distilled water. The whole mixture is left in a 23 ml Teflon body placed in a Paar metal bomb for 3 days at 130° C. The solid is then filtered off and washed with acetone.

The solid (200 mg) is then suspended in 100 ml of distilled water at reflux with stirring for 3 h in order to remove the trimesic acid remaining in the pores. The solid is then recovered by hot filtration.

### Characteristic Data for the Iron Carboxylate Solid MIL-100(Fe):

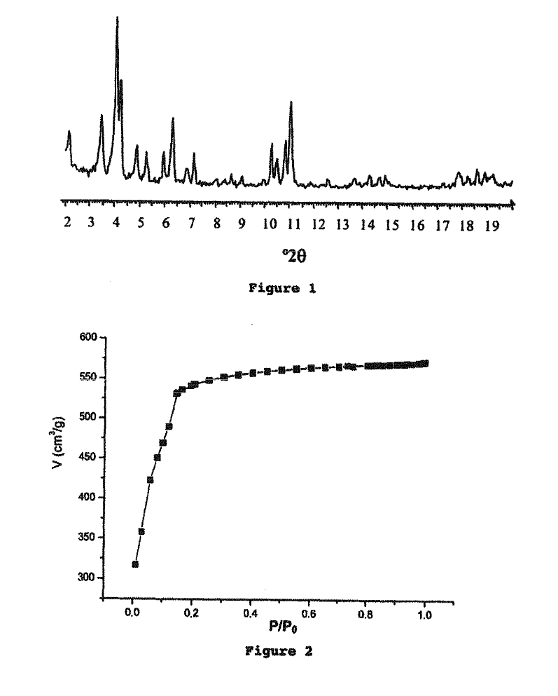

The analysis of the crystal structure of the solid MIL-100(Fe) by X-ray diffraction gives the X-ray diffractogram represented in FIG. 1.

The characteristics of the crystal structure are the following:
the space group is Fd-3m (No. 227).
The unit cell parameters are: a=73.1 Å, unit cell volume V=393000 Å³.

The nitrogen absorption isotherm at 77 K of the solid MIL-100(Fe) (at the pressure P₀=1 atm) is given in FIG. 2. The specific surface area (Langmuir) of this solid is close to 2900 m²·g⁻¹.

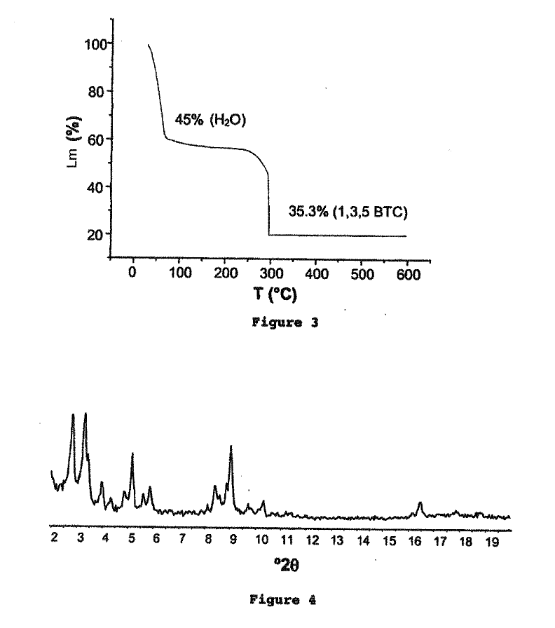

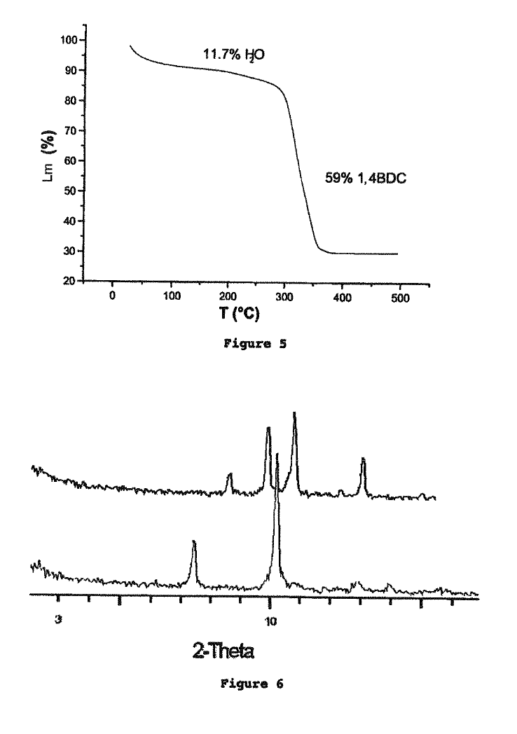

The curve resulting from the thermogravimetric analysis of the compound MIL-100(Fe) is given in FIG. 3. This diagram represents the loss of mass Lm (as %) as a function of the temperature T (in ° C.).

The table below gives the elemental analysis of the solid MIL-100(Fe) or Fe₃O[C₆H₃—(CO₂)₃]₂.X.nH₂O in the case where X═F.

TABLE 1

| | Element (% by mass) | | |
|---|---|---|---|
| | % Iron | % Carbon | % Fluorine |
| MIL-100(Fe) | 13.8 | 23.5 | 1.3 |

b) MIL-101(Fe) or Fe₃O[C₆H₄—(CO₂)₂]₃.X.nH₂O (X═F, Cl, OH)

### Synthesis of the Solid MIL-101(Fe):

0.27 g (1 mmol) of FeCl₃.6H₂O and 249 mg (1.5 mmol) of 1,4-benzenedicarboxylic acid (1,4-BDC, sold by the company Aldrich, 99%) are dispersed in 10 ml of dimethylformamide (DMF, sold by the company Fluka, 98%). The mixture is left for 12 hours at 100° C. in a 23 ml Teflon body placed in a Paar metal bomb. The solid is then filtered off and washed with acetone.

### Characteristic Data for the Solid MIL-101(Fe):

The X-ray diffractogram of the solid MIL-101(Fe) is represented in FIG. 4.

The characteristics of the crystal structure are the following:
the space group is Fd-3m (No. 227).
the unit cell parameters of the solid MIL-101(Fe) at 298 K are: a=89.0 Å; unit cell volume V=707000 Å³.

The theoretical elemental composition of the dry solid (with X═F) is the following: Fe 24.2%; C 41.4%; F 2.7%; H 1.7%.

c) MIL-88A(Fe) or Fe₃O[C₂H₂—(CO₂)₂]₃.X.nH₂O (X═F, Cl, OH)

### Synthesis of the Solid MIL-88A(Fe):

0.27 g (1 mmol) of FeCl₃.6H₂O (sold by the company Alfa Aesar, 98%) and 116 mg (1 mmol) of fumaric acid (Aldrich, 99%) are dispersed in 5 ml of dimethylformamide (DMF, Fluka, 98%) with 0.4 ml of 2M NaOH (Alfa Aesar, 98%). The mixture is left in a 23 ml Teflon body placed in a Paar metal bomb for 12 hours at 100° C. The solid is then filtered off and washed with acetone.

The solid (200 mg) is then suspended in 100 ml of distilled water with stirring for 12 h in order to remove the solvent remaining in the pores. The solid is then recovered by filtration.

### Characteristic Data for the Solid MIL-88A(Fe):

The analysis of the crystal structure of the solid gives the characteristics listed in the following table:

TABLE 2
unit cell parameters of the solid MIL-88A, dry
and hydrated

| | | | Unit cell | Pore | |
|---|---|---|---|---|---|
| | a | c | volume | size | Space |
| Phase | (Å) | (Å) | (Å³) | (Å) | group |
| MIL-88A dry | 9.25 | 15.30 | 1135 | — | P-62c |
| MIL-88A | 13.9 | 12.66 | 2110 | 6-7 | P-62c |
| hydrated | | | | | |
| (H₂O) | | | | | |

The X-ray diffractogram is given in FIG. 6.

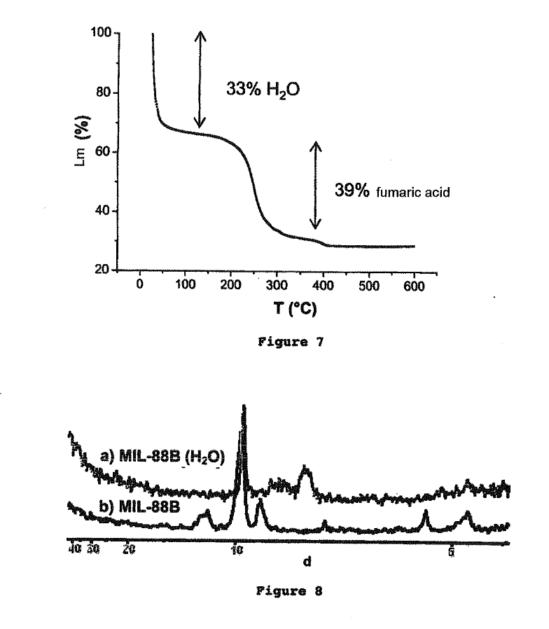

The results of the thermogravimetric analysis of the hydrated compound MIL-88A (in air, at a heating rate of 2° C./minute) are represented in FIG. 7. The loss of mass LM (as %) is represented as a function of the temperature T (in ° C.).

The compound MIL-88A does not exhibit a surface area (greater than 20 m²/g) accessible to nitrogen at 77 K, since the dry structure has a pore size which is too small to incorporate nitrogen N₂.

The elemental analysis is given in the table below:

TABLE 3

Element
(% by mass)

| | % Iron | % Carbon |
|---|---|---|
| MIL-88A (crude) | 21.8 | 24.0 |

d) MIL-88B(Fe) or Fe₃O[C₆H₄—(CO₂)₂]₃.X.nH₂O═F, Cl, OH)

### Synthesis of the Solid MIL-88B(Fe):

0.27 g (1 mmol) of FeCl₃.6H₂O (Alfa Aesar, 98%) and 116 mg (1 mmol) of 1,4-benzenedicarboxylic acid (Aldrich, 98%) are dispersed in 5 ml of dimethylformamide (DMF, Fluka, 98%) with 0.4 ml of 2M sodium hydroxide (Alfa Aesar, 98%). The mixture is left in a 23 ml Teflon body placed in a Paar metal bomb for 12 hours at 100° C. The solid is then filtered off and washed with acetone.

200 mg of the solid are suspended in 100 ml of distilled water with stirring for 12 h in order to remove the solvent remaining in the pores. The solid is then recovered by filtration.

### Characteristic Data for the Solid MIL-88B(Fe):

The analysis of the crystal structure of the solid gives the characteristics listed in the following table:

TABLE 4
unit cell parameters of the solid MIL-88B, dry
and hydrated

| | | | Unit cell | Pore | |
|---|---|---|---|---|---|
| | a | c | volume | size | Space |
| Phase | (Å) | (Å) | (Å³) | (Å) | group |
| MIL-88B dry | 9.6 | 19.1 | 1500 | <3 | P-62c |
| MIL-88B | 15.7 | 14.0 | 3100 | 9 | P-62c |
| hydrated | | | | | |
| (EtOH) | | | | | |

FIG. 8 represents the X-ray diffractograms of the dry solid and of the hydrated solid.

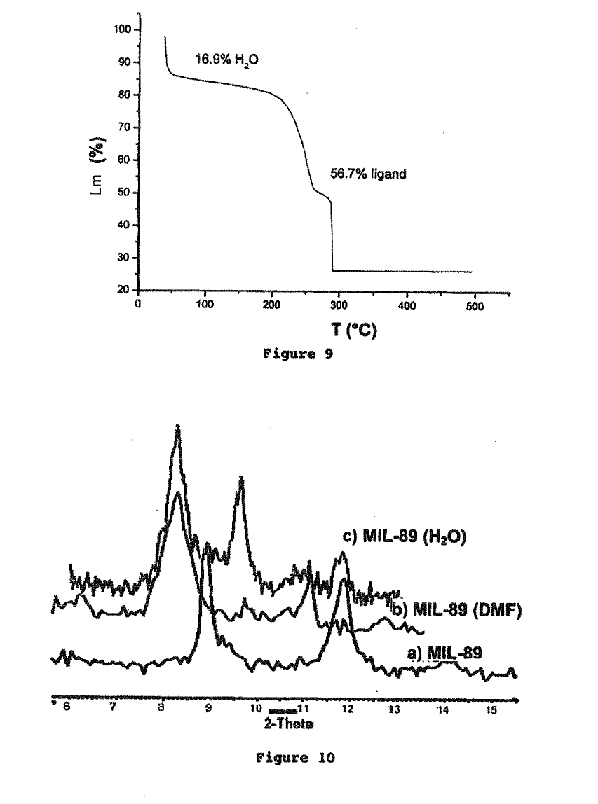

The results of the thermogravimetric analysis of the hydrated compound MIL-88B (in air, at a heating rate of 2° C./minute) are represented in FIG. 9. The loss of mass Lm (as %) is represented as a function of the temperature T (in ° C.).

The compound MIL-88B does not exhibit a surface area (greater than 20 m²/g) accessible to nitrogen at 77 K, since the dry structure has a pore size which is too small to incorporate nitrogen N₂.

e) MIL-89(Fe) or Fe₃O[C₄H₄—(CO₂)₂]₃—X.nH₂O (X═F, Cl, OH)

### Synthesis of the Solid MIL-89(Fe):

172 mg (1 mmol) of iron acetate (prepared according to the synthesis described by Dziobkowski et al., Inorg. Chem., 1982, 20, 671 [ref. 14]) and 150 mg (1 mmol) of muconic acid (Fluka, 97%) are dispersed in 10 ml of methanol (Fluka, 98%) with 0.35 ml of 2M sodium hydroxide (Alfa Aesar, 98%). The mixture is left in a 23 ml Teflon body placed in a Paar metal bomb for 3 days at 100° C. The solid is then filtered off and washed with acetone.

200 mg of the solid are suspended in 100 ml of distilled water with stirring for 12 h in order to remove the solvent remaining in the pores. The solid is then recovered by filtration.

### Characteristic Data for the Solid MIL-89(Fe):

FIG. 10 represents the X-ray diffractograms a), b) and c), respectively, of the dry solid MIL-89(Fe), of the solid MIL-89(Fe) solvated with DMF and of the hydrated solid MIL-89(Fe).

The compound MIL-89(Fe) does not exhibit a surface area (greater than 20 m²/g) accessible to nitrogen at 77 K, since the dry structure has a pore size which is too small to incorporate nitrogen N₂.

f) MIL-88C(Fe) or Fe₃O[C₁₀H₄—(CO₂)₂]₃.X.nH₂O (X═F, Cl, OH)

### Synthesis of the Solid MIL-88C(Fe):

172 mg (1 mmol) of iron acetate (synthesized according to example 2) and 140 mg (1 mmol) of 2,6-naphthalenedicarboxylic acid (Aldrich, 95%) are dispersed in 5 ml of dimethylformamide (DMF, Fluka, 98%). The mixture is left in a 23 ml Teflon body placed in a Paar metal bomb for 3 days at 150° C. with a temperature rise stage of 12 hours and a temperature drop stage of 24 hours. The solid is recovered by filtration. The solid is dried at 150° C. under air for 15 hours.

### Characteristic Data for the Solid MIL-88C(Fe):

The analysis of the crystal structure of the solid gives the characteristics listed in the following table:

TABLE 5
unit cell parameters of the solid MIL-88C, dry
and solvated

| | | | Unit cell | Pore | |
|---|---|---|---|---|---|
| | a | c | volume | size | Space |
| Phase | (Å) | (Å) | (Å³) | (Å) | group |
| MIL-88C dry | 9.9 | 23.8 | 2020 | 3 | P-62c |
| MIL-88C | 18.7 | 18.8 | 5600 | 13 | P-62c |
| solvated | | | | | |
| (Pyridine) | | | | | |

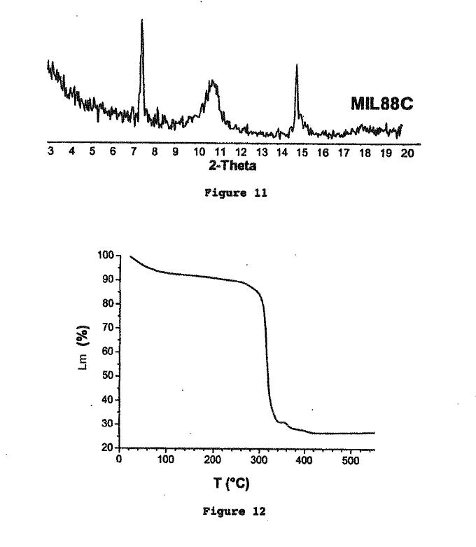

FIG. 11 represents the X-ray diffractogram of the solid MIL-88C.

The results of the thermogravimetric analysis of the crude synthetic compound MIL-88C (in air, at a heating rate of 2° C./minute) are represented in FIG. 12.

This compound does not exhibit a surface area (greater than 20 m²/g) accessible to nitrogen at 77 K, since the dry structure has a pore size which is too small to incorporate nitrogen N₂.

g) MIL-88D(Fe) or Fe₃O[C₁₂H₈—(CO₂)₂]₃.X.nH₂O (X═F, Cl, OH)

### Synthesis of the Solid MIL-88D(Fe):

270 mg (1 mmol) of FeCl₃.6H₂O (Alfa Aesar, 98%) and 140 mg (0.6 mmol) of 4,4′-biphenyldicarboxylic acid (Fluka, 95%) are dispersed in 5 ml of dimethylformamide (DMF, Aldrich, 99%). The mixture is left in a 23 ml Teflon body placed in a Paar metal bomb for 12 hours at 100° C. with a temperature rise stage of one hour and a temperature drop stage of one hour. The solid is recovered by filtration.

The solid is then dried at 150° C. in air for 15 hours.

### Characteristic Data for the Solid MIL-88D(Fe):

The analysis of the crystal structure of the solid gives the characteristics listed in the following table:

TABLE 6
unit cell parameters of the solid MIL-88D, dry
and solvated (pyridine)

| | | | Unit cell | Pore | |
|---|---|---|---|---|---|
| | a | c | volume | size | Space |
| Phase | (Å) | (Å) | (Å³) | (Å) | group |
| MIL-88D dry | 10.1 | 27.8 | 2480 | <3 | P-62c |
| MIL-88D | 20.5 | 22.4 | 8100 | 16 | P-62c |
| solvated | | | | | |
| (pyridine) | | | | | |

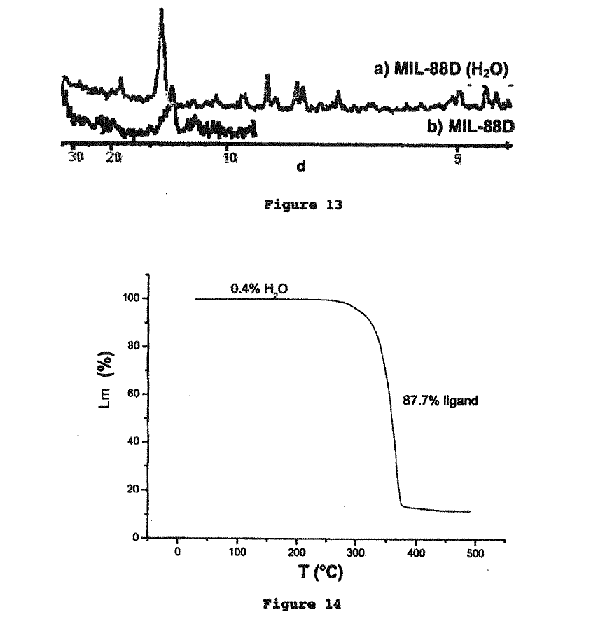

FIG. 13 represents the X-ray diffractogram of the crude solid MIL-88D (bottom curve (b)) and the hydrated solid MIL-88D (top curve (a)).

The results of the thermogravimetric analysis of the hydrated compound MIL-88D(Fe) (in air, at a heating rate of 2° C./minute) are represented in FIG. 14 (loss of mass Lm as a function of the temperature T).

This compound does not exhibit a surface area (greater than 20 m²/g) accessible to nitrogen at 77 K, since the dry structure has a pore size which is too small to incorporate nitrogen N₂.

h) MIL-88B-NO₂ (Fe) or Fe₃O[C₆H₃NO₂— (CO₂)₂]₃.X.nH₂O (X═F, Cl, OH)

### Synthesis of the Solid MIL-88B-NO₂(Fe):

0.27 g (1 mmol) of FeCl₃.6H₂O (Alfa Aesar, 98%) and 211 mg (1 mmol) of 2-nitroterephthalic acid (Acros, 99%) are dispersed in 5 ml of distilled water. The mixture is left in a 23 ml Teflon body placed in a Paar metal bomb for 12 hours at 100° C. The solid is recovered by filtration.

200 mg of the solid are suspended in 10 ml of absolute ethanol in a 23 ml Teflon body placed in a Paar metal bomb for 12 hours at 100° C. in order to remove the acid remaining in the pores. The solid is then recovered by filtration and dried at 100° C.

Characteristic Data for the Solid MIL-88b-NO₂(Fe):

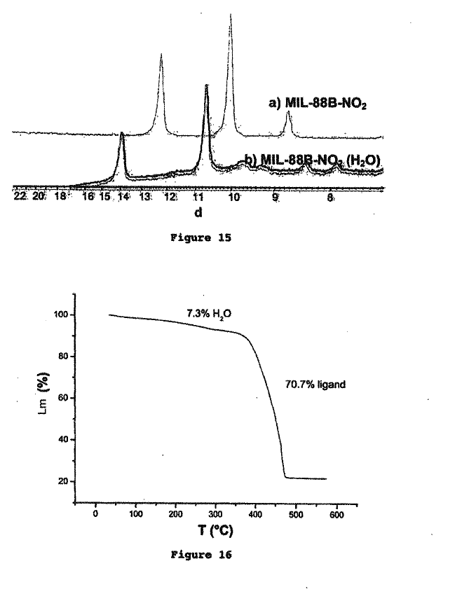

FIG. 15 represents the X-ray diffractogram of the crude solid MIL-88B-NO₂ (top curve (a)) and the hydrated solid MIL-885-NO₂ (bottom curve (b)).

The results of the thermogravimetric analysis (in air, at a heating rate of 2° C./minute) of the compound MIL-88B-NO₂(Fe), after washing and drying, are represented in FIG. 16. The loss of mass Lm (as %) is represented as a function of the temperature T (in ° C.).

This compound does not exhibit a surface area (greater than 20 m²/g) accessible to nitrogen at 77 K, since the dry structure has a pore size which is too small to incorporate nitrogen N₂.

The elemental analysis is given in the table below:

TABLE 7

| | Element (% by mass) | | |
|---|---|---|---|
| | % Iron | % Carbon | % Nitrogen |
| MIL-88B-NO₂ | 20.6 | 39.3 | 4.6 |

i) MIL-888-2OH(Fe) or Fe₃O [C₆H₂ (OH)₂— (CO₂)₂]₃X.nH₂O (X═F, Cl, OH)

### Synthesis of the Solid MIL-88B-2OH(Fe):

354 mg (1 mmol) of Fe(ClO₄)₃.xH₂O (Aldrich, 99%) and 198 mg (1 mmol) of 2,5-dihydroxyterephthalic acid (obtained by hydrolysis of the corresponding diethyl ester, Aldrich, 97%) are dispersed in 5 ml of DMF (Fluka, 98%). The mixture is left in a 23 ml Teflon body placed in a Paar metal bomb for 12 hours at 85° C. The solid is recovered by filtration.

In order to remove the acid remaining in the pores, the product is calcined at 150° C. under vacuum for 15 hours.

### Characteristic Data for the Solid MIL-888-2OH(Fe):

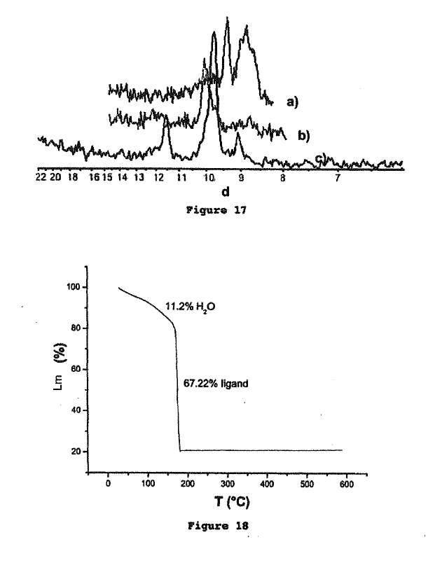

FIG. 17 represents the X-ray diffractogram of the solid MIL-88B-2OH crude (bottom curve (c)), hydrated (middle curve (b)) and dried under vacuum (top curve (a)).

The results of the thermogravimetric analysis (in air, at a heating rate of 2° C./minute) of the compound MIL-88B-2OH(Fe), after washing and drying, are represented in FIG. 18. The loss of mass Lm (as %) is represented as a function of the temperature T (in ° C.).

This compound does not exhibit a surface area (greater than 20 m²/g) accessible to nitrogen at 77 K, since the dry structure has a pore size which is too small to incorporate nitrogen N₂.

The elemental analysis is given in the table below:

TABLE 8

Element
(% by mass)

| | % Iron | % Carbon |
|---|---|---|
| MIL-88B-2OH | 15.4 | 36.5 |

j) MIL-88B-NH₂(Fe) or Fe₃O[C₆H₃NH₂— (CO₂)₂]₃.X.nH₂O (X═F, Cl, OH)

### Synthesis of the Solid MIL-88B-NH₂(Fe):

0.27 g (1 mmol) of FeCl₃.6H₂O (Alfa Aesar, 98%) and 180 mg (1 mmol) of 2-aminoterephthalic acid (Fluka, 98%) are dispersed in 5 ml of absolute ethanol. The mixture is left in a 23 ml Teflon body placed in a Paar metal bomb for 3 days at 100° C. The solid is recovered by filtration.

In order to remove the acid remaining in the pores, the solid is calcined at 200° C. for 2 days.

### Characteristic Data for the Solid MIL-88B-NH₂(Fe):

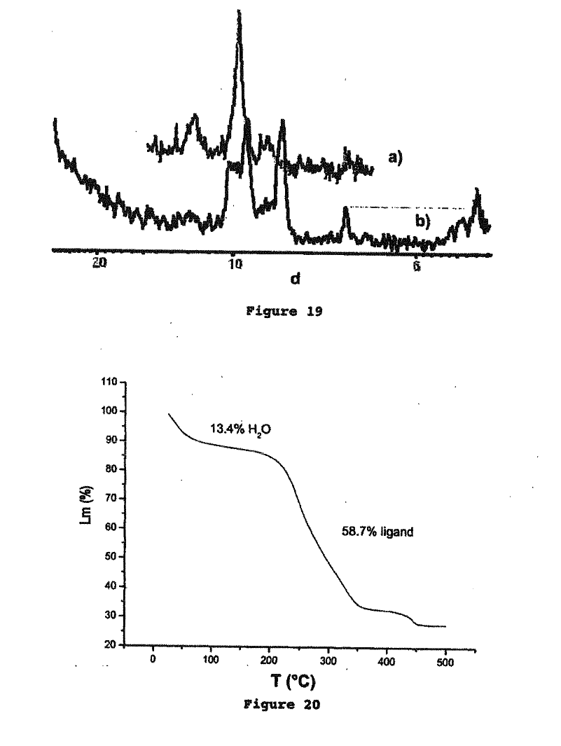

FIG. 19 represents the X-ray diffractogram of the crude solid MIL-88B-NH₂ (bottom curve (b)) and the solid MIL-88B-NH₂ dried under vacuum (top curve (a)).

The results of the thermogravimetric analysis (in air, at a heating rate of 2° C./minute) of the hydrated solid MIL-88B-NH₂(Fe) are represented in FIG. 20.

This compound does not exhibit a surface area (greater than 20 m²/g) accessible to nitrogen at 77 K, since the dry structure has a pore size which is too small to incorporate nitrogen N₂.

k) MIL-88B-CH₃(Fe) or Fe₃O [C₆H₃CH₃— (CO₂)₂]₃.X.nH₂O (X═F, Cl, OH)

### Synthesis of the Solid MIL-88B-CH₃(Fe):

354 mg (1 mmol) of Fe(ClO₄)₃.xH₂O (Aldrich, 99%) and 180 mg (1 mmol) of 2-methylterephthalic acid (prepared according to the synthesis described by Anzalone et al., J. Org. Chem. 1985, 50, 2128 [ref. 15]) are dispersed in 5 ml of methanol (Fluka, 99%). The mixture is left in a 23 ml Teflon body placed in a Paar metal bomb for 3 days at 100° C. The solid is recovered by filtration.

200 mg of the solid are suspended in 10 ml of DMF with stirring at ambient temperature in order to exchange the acid present in the pores with DMF, and then the DMF is removed by heating at 150° C. under vacuum for 12 hours.

### Characteristic Data for the Solid MIL-88B-CH₃(Fe):

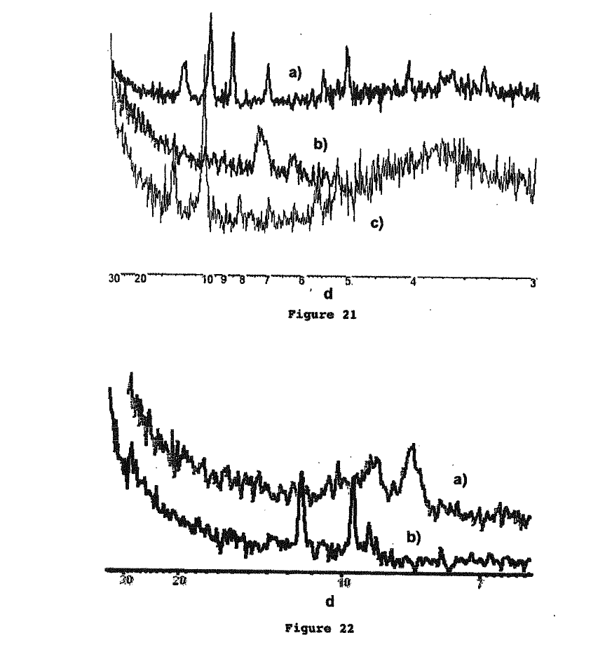

FIG. 21 represents the X-ray diffractogram of the solid MIL-88B-CH₃ crude (top curve (a)), hydrated (middle curve (b)) and solvated with DMF (bottom curve (c)).

This compound does not exhibit a surface area (greater than 20 m²/g) accessible to nitrogen at 77 K, since the dry structure has a pore size which is too small to incorporate nitrogen N₂.

1) MIL-88B-Cl (Fe) or Fe₃O[C₆H₃Cl—(CO₂)₂]₃.X.nH₂O (X═F, Cl, OH)

### Synthesis of the Solid MIL-88B-Cl(Fe):

354 mg (1 mmol) of Fe (ClO₄)₃.xH₂O (Aldrich, 99%) and 200 mg (1 mmol) of 2-chloroterephthalic acid (synthesized according to synthesis A of example 3) are dispersed in 10 ml of DMF with 0.1 ml of 5M HF (SDS, 50%) and 0.1 ml of 1M HCl (Aldrich, 37%). The mixture is left in a 23 ml Teflon body placed in a Paar metal bomb for 5 days at 100° C. The solid is recovered by filtration.

The solid obtained is calcined at 150° C. under vacuum.

### Characteristic Data for the Solid MIL-88B-Cl (Fe):

FIG. 21 represents the X-ray diffractogram of the solid MIL-88B-Cl crude (top curve (a)), hydrated (middle curve (b)) and solvated with DMF (bottom curve (c)).

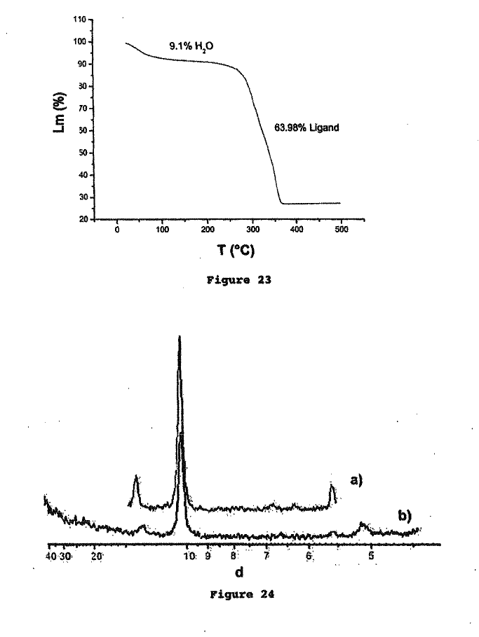

The thermogravimetric analysis (in air, at a heating rate of 2° C./minute) of the hydrated solid MIL-88B-Cl(Fe) is represented in FIG. 23.

This compound does not exhibit a surface area (greater than 20 m²/g) accessible to nitrogen at 77 K, since the dry structure has a pore size which is too small to incorporate nitrogen N₂.

m) MIL-88B-4-CH₃(Fe) or Fe₃O[C₆(CH₃)₄—(CO₂)₂]₃.X.nH₂O (X═F, Cl, OH)

### Synthesis of the Solid MIL-88B-4-CH₃ (Fe):

0.27 g (1 mmol) of FeCl₃.6H₂O (Alfa Aesar, 98%) and 222 mg (1 mmol) of 1,4-tetramethylterephthalic acid (Chem Service, 95%) are dispersed in 10 ml of DMF (Fluka, 98%) with 0.4 ml of 2M sodium hydroxide (Alfa Aesar, 98%). The mixture is left in a 23 ml Teflon body placed in a Paar metal bomb for 12 hours at 100° C. The solid is recovered by filtration.

200 mg of the solid are suspended in 100 ml of water with stirring at ambient temperature for 12 hours in order to remove the acid remaining in the pores. The solid is then recovered by filtration.

### Characteristic Data for the Solid MIL-88B-4-CH₃ (Fe):

FIG. 24 represents the X-ray diffractogram of the crude solid (bottom curve (b)) and of the hydrated solid (top curve (a)).

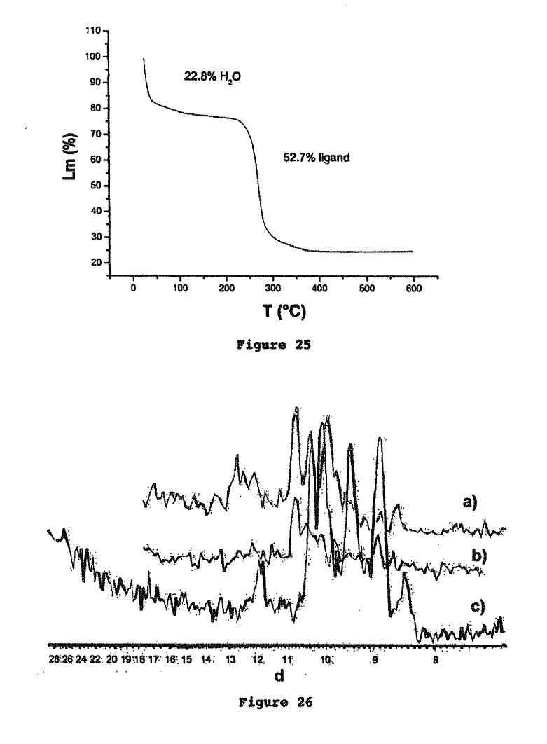

The thermogravimetric analysis (in air, at a heating rate of 2° C./minute) of the hydrated solid MIL-88B-4-CH₃(Fe) is represented in FIG. 25.

This compound exhibits a surface area, of about 1200 m²/g (Langmuir), accessible to nitrogen at 77 K, since the dry structure has a pore size which is sufficient (6-7 Å) to incorporate nitrogen N₂.

n) MIL-88B-4F (Fe) or Fe₃O[C₆F₄—(CO₂)₂]₃.X.nH₂O (X═F, Cl, OH)

### Synthesis of the Solid MIL-88B-4F (Fe):

270 mg (1 mmol) of FeCl₃.6H₂O (Alfa Aesar, 98%) and 230 mg (1 mmol) of tetrafluoroterephthalic acid (Aldrich, 98%) are dispersed in 10 ml of distilled water. The mixture is left in a 23 ml Teflon body placed in a Paar metal bomb for 12 hours at 85° C. The solid is recovered by filtration.

200 mg of the solid are suspended in 20 ml of water with stirring at ambient temperature for 2 hours in order to remove the acid remaining in the pores. The solid is then recovered by filtration.

### Characteristic Data for the Solid MIL-88B-4F (Fe):

FIG. 26 represents the X-ray diffractogram of the crude solid (bottom curve (c)), of the hydrated solid (curve (b)) and of the solid solvated with ethanol (top curve (a)).

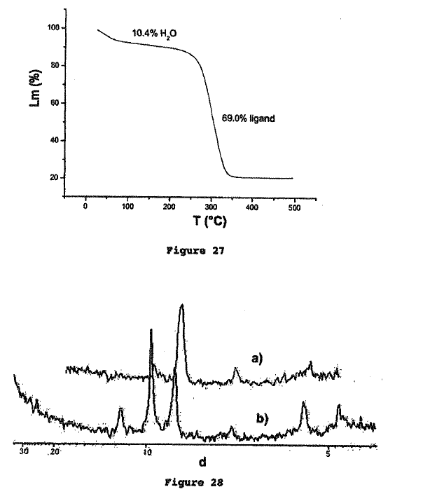

The thermogravimetric analysis (in air, at a heating rate of 2° C./minute) of the hydrated solid MIL-88B-4F(Fe) is represented in FIG. 27.

This compound does not exhibit a surface area (greater than 20 m²/g) accessible to nitrogen at 77 K, since the dry structure has a pore size which is too small to incorporate nitrogen N₂.

o) MIL-88B-Br (Fe) or Fe₃O[C₆H₃Br—(CO₂)₂]₃.X.nH₂O (X═F, Cl, OH)

### Synthesis of the Solid MIL-88B-Br (Fe):

270 mg (1 mmol) of FeCl₃.6H₂O (Alfa Aesar, 98%) and 250 mg (1 mmol) of 2-bromoterephthalic acid (Fluka, 95%) are dispersed in 10 ml of DMF (Fluka, 98%) with 0.2 ml of 5M hydrofluoric acid (SDS, 50%). The mixture is left in a 23 ml Teflon body placed in a Paar metal bomb for 12 hours at 150° C. The solid is recovered by filtration.

In order to remove the acid remaining in the pores, the solid is calcined at 150° C. under vacuum for 15 hours.

### Characteristic Data for the Solid MIL-88B-Br (Fe):

FIG. 28 represents the X-ray diffractogram of the crude solid (bottom curve (b)) and of the hydrated solid (top curve (a)).

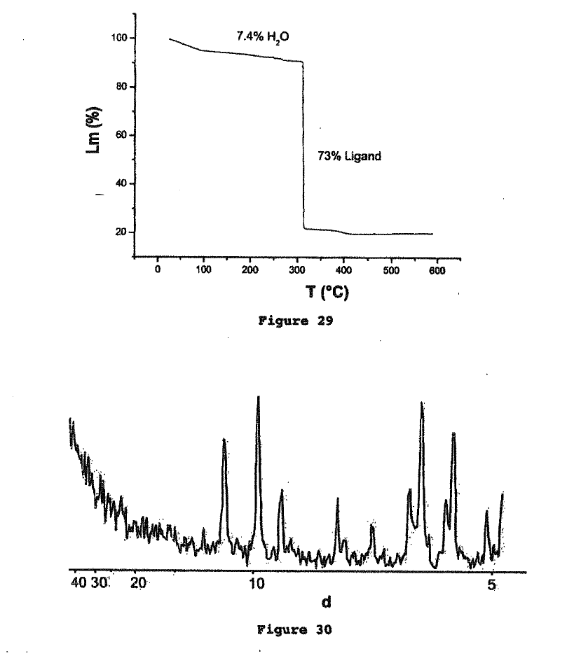

The thermogravimetric analysis (in air, at a heating rate of 2° C./minute) of the hydrated solid MIL-88B-Br(Fe) is represented in FIG. 29.

This compound does not exhibit a surface area (greater than 20 m²/g) accessible to nitrogen at 77 K, since the dry structure has a pore size which is too small to incorporate nitrogen N₂.

p) MIL-88E (Pyr) (Fe) or Fe₃O[C₄H₃N₂—(CO₂)₂]₃.X.nH₂O (X═F, Cl, OH)

### Synthesis of the Solid MIL-88E (Fe):

270 mg (1 mmol) of FeCl₃.6H₂O (Alfa Aesar, 98%) and 204 mg (1 mmol) of 2,5-pyrazinedicarboxylic acid (Aldrich, 98%) are dispersed in 5 ml of DMF (Fluka, 98%) with 0.05 ml of 5M HF (SDS, 50%). The mixture is left in a 23 ml Teflon body placed in a Paar metal bomb for 3 days at 100° C. The solid is recovered by filtration.

### Characteristic Data for the Solid MIL-88E (Fe):

FIG. 30 represents the X-ray diffractogram of the crude synthetic solid MIL-88E(Fe).

This compound does not exhibit a surface area (greater than 20 m²/g) accessible to nitrogen at 77 K, since the dry structure has a pore size which is too small to incorporate nitrogen N₂.

q) MIL-88F (Thio) (Fe) or Fe₃O[C₄H₂S—(CO₂)₂]₃.X.nH₂O (X═F, Cl, OH)

### Synthesis of the Solid MIL-88F(Fe):

354 mg (1 mmol) of Fe(ClO₄)₃.xH₂O (Aldrich, 99%) and 258 mg (1 mmol) of 2,5-thiophenedicarboxylic acid (Aldrich, 99%) are dispersed in 2.5 ml of DMF (Fluka, 98%) with 0.1 ml of 5M HF (SDS, 50%). The mixture is left in a 23 ml Teflon body placed in a Paar metal bomb for 3 days at 100° C. The solid is recovered by filtration.

200 mg of the solid are suspended in 100 ml of water with stirring at ambient temperature for 12 hours in order to remove the acid remaining in the pores. The solid is then recovered by filtration.

### Characteristic Data for the Solid MIL-88F(Fe):

FIG. 31 represents the X-ray diffractograms of the crude solid (bottom curve (b)) and of the hydrated solid (top curve (a)).

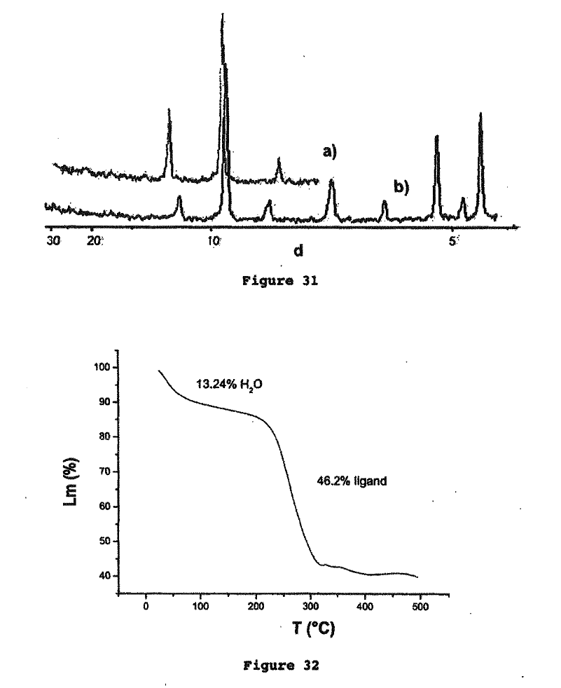

The thermogravimetric analysis (in air, at a heating rate of 2° C./minute) of the hydrated solid MIL-88F(Fe) is represented in FIG. 32.

This compound does not exhibit a surface area (greater than 20 m²/g) accessible to nitrogen at 77 K, since the dry structure has a pore size which is too small to incorporate nitrogen N₂.

r) MIL-88D-4-CH₃ (Fe) or Fe₃O[C₁₂H₄(CH₃)₄—(CO₂)₃.X.nH₂O (X═F, Cl, OH)

### Synthesis of the Solid MIL-88D-4-CH₃ (Fe):

354 mg (1 mmol) of Fe(ClO₄)₃.xH₂O (Aldrich, 99%) and 298 mg (1 mmol) of tetramethylbiphenyl-4,4′-dicarboxylic acid (synthesized according to synthesis B described in example 3) are dispersed in 5 ml of DMF (Fluka, 98%) with 0.2 ml of 2M sodium hydroxide (Alfa Aesar, 98%). The mixture is left in a 23 ml Teflon body placed in a Paar metal bomb for 12 hours at 100° C. The solid is recovered by filtration.

200 mg of the solid are suspended in 10 ml of DMF with stirring at ambient temperature for 2 hours in order to exchange the acid remaining in the pores. The solid is then recovered by filtration, and the DMF present in the pores is removed by calcination at 150° C. under vacuum for 15 hours.

### Characteristic Data for the Solid MIL-88D-4-CH₃(Fe):

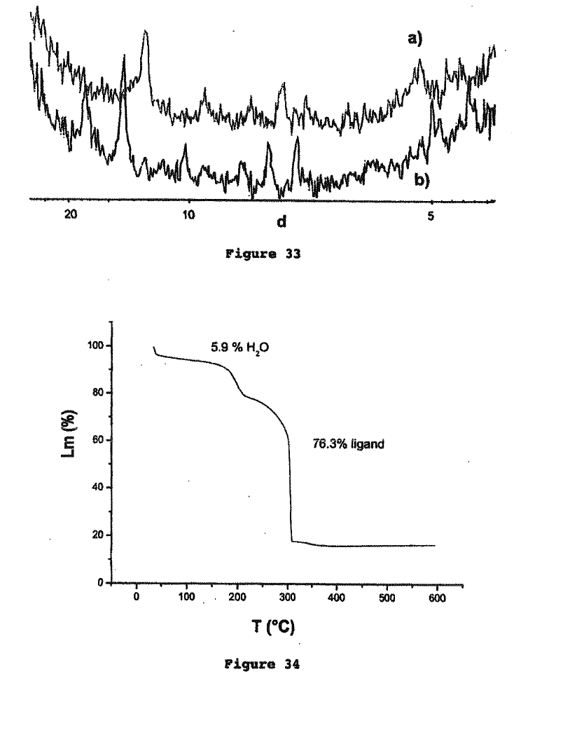

FIG. 33 represents the X-ray diffractograms of the crude solid (bottom curve (b)) and of the hydrated solid (top curve (a)).

The thermogravimetric analysis (in air, at a heating rate of 2° C./minute) of the hydrated solid MIL-88D-4-CH₃(Fe) is represented in FIG. 34.

This compound does not exhibit a surface area (greater than 20 m²/g) accessible to nitrogen at 77 K, since the dry structure has a pore size which is too small to incorporate nitrogen N₂.

s) MIL-88D-2CH₃(Fe) or Fe₃O(C₁₂H₆ (CH₃)₂—(CO₂)₃.X.nH₂O (X═F, Cl, OH)

### Synthesis of the Solid MIL-88D-2CH₃(Fe):

270 mg (1 mmol) of FeCl₃.6H₂O (Alfa Aesar, 98%) and 268 mg (1 mmol) of dimethylbiphenyl-4,4′-dicarboxylic acid (synthesized according to synthesis C described in example 3) are dispersed in 5 ml of DMF (Fluka, 98%) with 0.25 ml of 5M HF (SDS, 50%). The mixture is left in a 23 ml Teflon body placed in a Paar metal bomb for 12 hours at 150° C. The solid is recovered by filtration.

In order to remove the acid remaining in the pores, the solid is calcined at 150° C. under vacuum for 15 hours.

### Characteristic Data for the Solid MIL-88D-2CH₂(Fe):

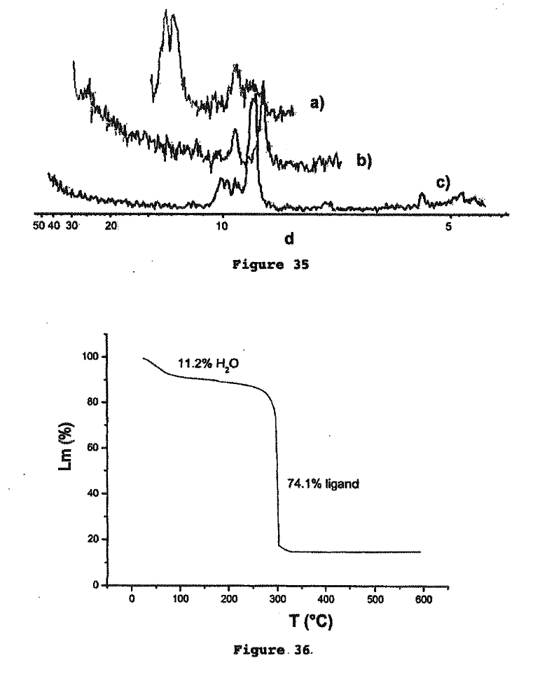

FIG. 35 represents the X-ray diffractograms of the crude solid (bottom curve (c)), of the hydrated solid MIL-88D-2CH₃(H₂O) (middle curve (b)) and of the solid in suspension in water MIL-88D-2CH₃(drop H₂O) (top curve (a)).

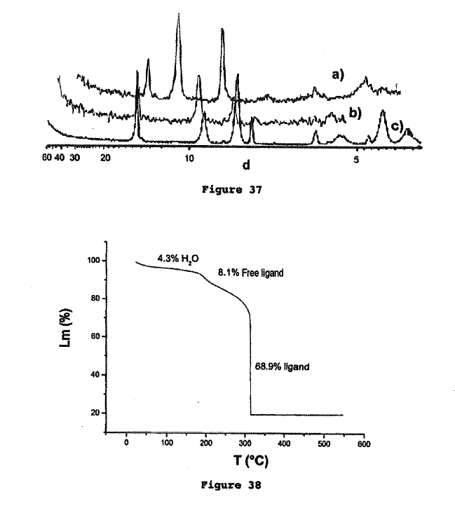

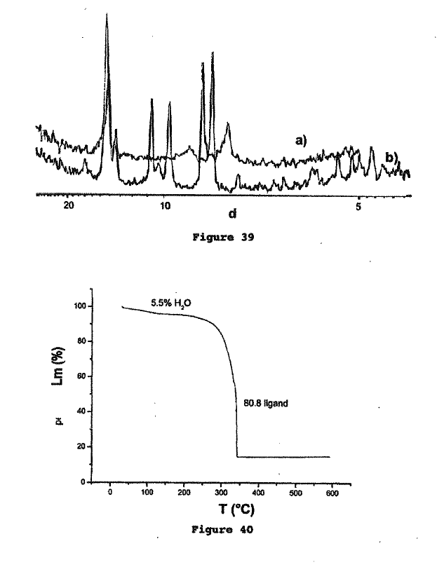

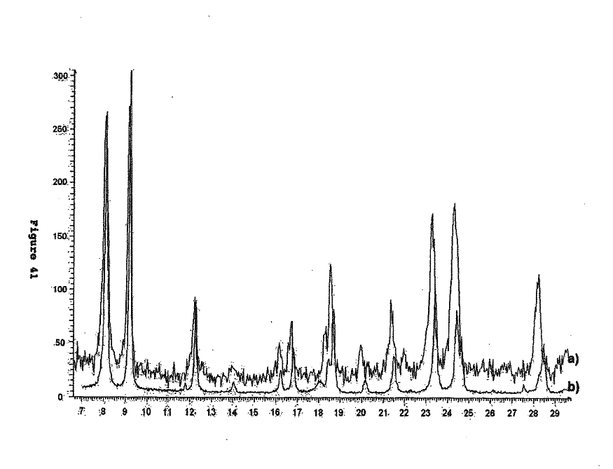

The thermogravimetric analysis (in air, at a heating rate of 2° C./minute) of the crude solid MIL-88D-2CH₃(Fe) is represented in FIG. 36.

This compound does not exhibit a surface area (greater than 20 m²/g) accessible to nitrogen at 77 K, since the dry structure has a pore size which is too small to incorporate nitrogen N₂.

t) MIL-88G (AzBz) (Fe) or Fe₃O[CH₁₂H₈N₂—(CO₂)₂]₃.X.nH₂O (X═F, Cl, OH)

### Synthesis of the Solid MIL-88G(Fe):

118 mg (0.33 mmol) of Fe(ClO₄)₃.xH₂O (Aldrich, 99%) and 90 mg (0.33 mmol) of 4,4′-azobenzenedicarboxylic acid (synthesized according to the method described by Ameerunisha et al., J. Chem. Soc. Perkin Trans. 2 1995, 1679 [ref. 16]) are dispersed in 15 ml of DMF (Fluka, 98%). The mixture is left in a 23 ml Teflon body placed in a Paar metal bomb for 3 days at 150° C. The solid is recovered by filtration.

200 mg of the solid are suspended in 10 ml of DMF with stirring at ambient temperature for 2 hours in order to exchange the acid remaining in the pores. The solid is then recovered by filtration and the DMF remaining in the pores is removed by calcination at 150° C. under vacuum for 15 hours.

### Characteristic Data for the Solid MIL-88G(Fe):

FIG. 37 represents the X-ray diffractograms of the crude solid MIL-88G (bottom curve (c)), of the solid solvated with DMF (middle curve (b)) and of the solid solvated with pyridine (top curve (a)).

The thermogravimetric analysis (in air, at a heating rate of 2° C./minute) of the crude solid MIL-88G(Fe) is represented in FIG. 38.

This compound does not exhibit a surface area (greater than 20 m²/g) accessible to nitrogen at 77 K, since the dry structure has a pore size which is too small to incorporate nitrogen N₂.

u) MIL-88G-2CL (AzBz-2Cl) (Fe) or Fe₃O[C₁₂H₆N₂Cl₂—(CO₂)₂]₃.X.nH₂O (X═F, Cl, OH)

### Synthesis of the Solid MIL-88G-2Cl (Fe):

177 mg (0.5 mmol) of Fe(ClO₄)₃.xH₂O (Aldrich, 99%) and 169 mg (0.5 mmol) of dichloro-4,4′-azobenzenedicarboxylic acid (synthesized according to synthesis D described in example 3) are dispersed in 15 ml of DMF (Fluka, 98%). The mixture is left in a 23 ml Teflon body placed in a Paar metal bomb for 12 hours at 150° C. The solid is recovered by filtration.

200 mg of the solid are suspended in 10 ml of DMF with stirring at ambient temperature for 2 hours in order to exchange the acid remaining in the pores. The solid is then recovered by filtration, and the DMF remaining in the pores is removed by calcination at 150° C. under vacuum for 15 hours.

### Characteristic Data for the Solid MIL-88G-2Cl (Fe):

FIG. 39 represents the X-ray diffractograms of the crude solid MIL-88G-2Cl (bottom curve (b)) and of the dry solid MIL-88G-2Cl (top curve (a)).

The thermogravimetric analysis (in air, at a heating rate of 2° C./minute) of the crude solid MIL-88G-2Cl (Fe) is represented in FIG. 40.

This compound does not exhibit a surface area (greater than 20 m²/g) accessible to nitrogen at 77 K, since the dry structure has a pore size which is too small to incorporate nitrogen N₂.

v) MIL-102(Fe) or Fe₆O₂X₂[C₁₀H₂—(CO₂)₄]₃.nH₂O (X═F, Cl . . . )

### Synthesis of the Solid MIL-102(Fe):

270 mg (1 mmol) of FeCl₃.6H₂O (Alfa Aesar, 98%) and 268 mg (1 mmol) of 1,4,5,8-naphthalenetetracarboxylic acid are dispersed in 5 ml of distilled water. The mixture is left in a 23 ml Teflon body placed in a Paar metal bomb for 15 hours at 100° C. The solid is recovered by filtration.

### Characteristic Data for the Solid MIL-102(Fe):

FIG. 41 represents the X-ray diffractograms of the crude solid MIL-102(Fe) (curve (a)) and of the solid MIL-102 (Cr) (curve (b)).

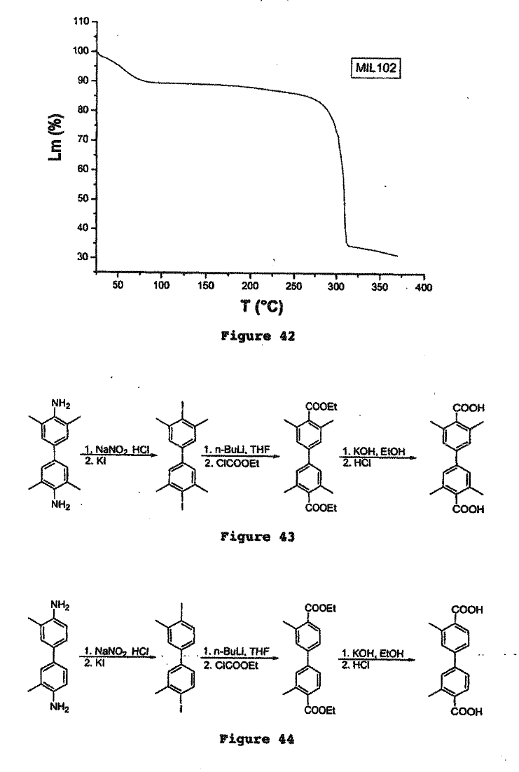

The thermogravimetric analysis (in air, at a heating rate of 2° C./min) of the crude solid MIL-102(Fe) is represented in FIG. 42.

This compound exhibits a low specific surface area (Langmuir surface area: 101 m²/g) with nitrogen at 77 K.

w) MIL-126(Fe) or Fe₆O₂X₂[C₁₀H₂—(CO₂)₄]₃.nH₂O (X═F, Cl . . . ) iron 4,4′-biphenyldicarboxylate

### Synthesis of the Solid MIL-126(Fe):

270 mg (1 mmol) of FeCl₃.6H₂O (Alfa Aesar, 98%) and 140 mg (0.6 mmol) of 4,4′-biphenyldicarboxylic acid (Fluka, 95%) are dispersed in 5 ml of dimethylformamide (DMF, Aldrich, 99%). The mixture is left in a 23 ml Teflon body placed in a Paar metal bomb for 12 hours at 150° C. with a temperature rise stage of 1 hour and a temperature drop stage of 1 hour. The solid is recovered by filtration.

The solid is then dried at 150° C. under a primary vacuum for 15 hours.

### Characteristic Data for the Solid MIL-126(Fe):

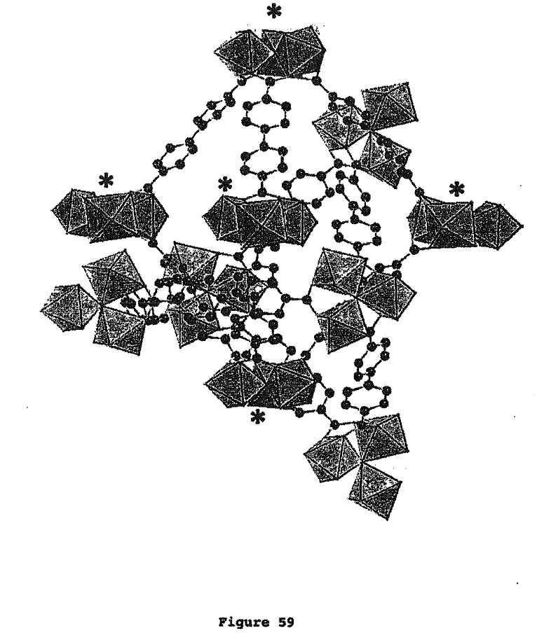

The crystallographic structure of the solid MIL-126(Fe) is an interpenetrated form of the MIL-88D(Fe) structure, i.e. it has two entangled crystalline subnetworks of MIL-88D type (FIG. 59).

The analysis of the crystal structure of the solid gives the characteristics listed in the following table.

TABLE 9
unit cell parameters of the solid MIL-126, dry
and solvated (dimethylformamide).

| | | | Unit | | |
|---|---|---|---|---|---|
| | | | cell | Pore | |
| | a | c | volume | size | Space |
| Phase | (Å) | (Å) | (Å³) | (Å) | group |
| MIL-126 dry | 19.5 | 35.3 | 13500 | 4 to 10 | P 41 21 2 |
| MIL-126 | 21.8 | 36.1 | 17200 | 5 to 11 | P 41 21 2 |
| solvated | | | | | |
| (DMF) | | | | | |

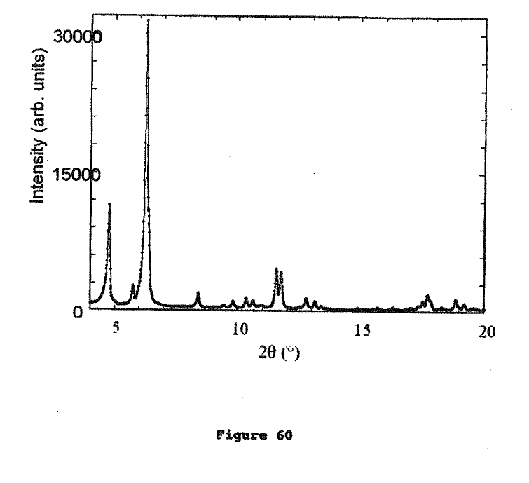

FIG. 60 represents the X-ray diffractogram of the crude solid MIL-126.

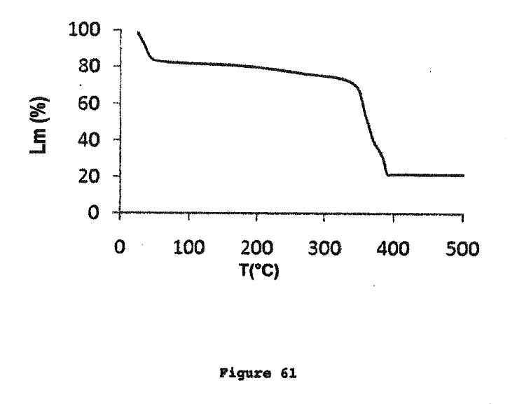

The results of the thermogravimetric analysis of the crude synthetic compound MIL-126(Fe) (in air, at a heating rate of 2° C./minute) are represented in FIG. 61 (loss of mass Lm as a function of the temperature T).

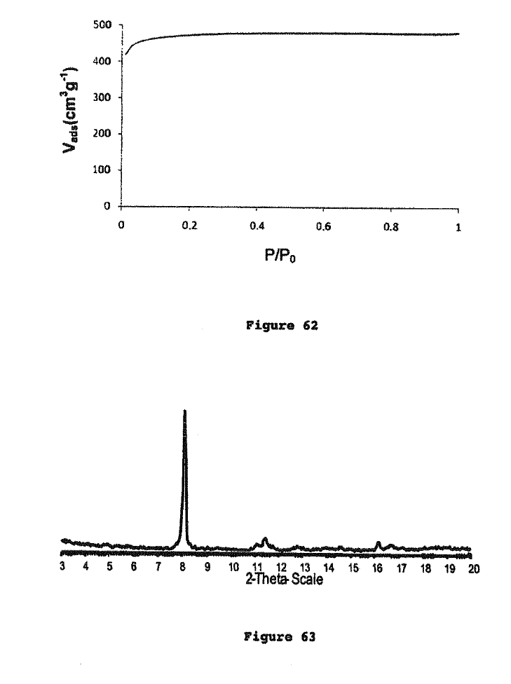

This compound exhibits a large surface area (Langmuir) (greater than 2100 m²/g) accessible to nitrogen at 77 K (FIG. 62).

y) iron 3,3′,5,5′-azobenzenetetracarboxylate or Fe₆O₂[C₁₂H₆N₂— (CO₂)₄]₃.X₂.nH₂O (X═F, Cl, OH)
Synthesis of the Solid Iron 3,3′,5,5′-azobenzenetetracarboxylate:

118 mg (0.3 mmol) of Fe(ClO₄)₃.nH₂O (Aldrich, 98%) and 119 mg (0.6 mmol) of 3,3′,5,5′-azobenzenetetracarboxylic acid (prepared according to the procedure indicated below in “synthesis I”) are dispersed in 5 ml of dimethylformamide (DMF, Aldrich, 99%) with the addition of 0.1 ml of 5M hydrofluoric acid (HF, SDS, 50%). The mixture is left in a 23 ml Teflon body placed in a Paar metal bomb for 3 days at 150° C. with a temperature rise stage of 1 hour. The solid is recovered by filtration.

The solid is then dried at 200° C. under a primary vacuum for 15 hours.

Synthesis I: 3,3′,5,5′-azobenzenetetracarboxylic acid

15 g of 2-nitroisophthalic acid (sold by the company Aldrich, 98%) and 50 g of sodium hydroxide are placed in 225 ml of distilled water, and heated to 50° C. with stirring. 100 g of glucose (Aldrich, 96%) dissolved in 150 ml of water are added. The mixture is stirred for 15 minutes, and is then sparged with air for 3 hours, at ambient temperature (20° C.). The disodium salt is recovered by filtration, washed with ethanol, and then redissolved in 120 ml of water. Hydrochloric acid (sold by the company Aldrich VWR, 37%) is added until a pH equal to 1 is obtained. The solid is recovered by filtration and dried under vacuum at 90° C.

Characteristic Data for the Solid Iron 3,3′,5,5′-azobenzenetetracarboxylate:

FIG. 63 represents the X-ray diffractogram for the crude solid iron 3,3′,5,5′-azobenzenetetracarboxylate. The phase, of cubic symmetry, is an isotype of that published by the group of Professor Eddaoudi with indium (space group Pa3) [51].

The results of the thermogravimetric analysis of the crude synthetic compound iron 3,3′,5,5′-azobenzenetetracarboxylate (in air, at a heating rate of 2° C./minute) are represented in FIG. 65 (loss of mass Lm as a function of the temperature T).

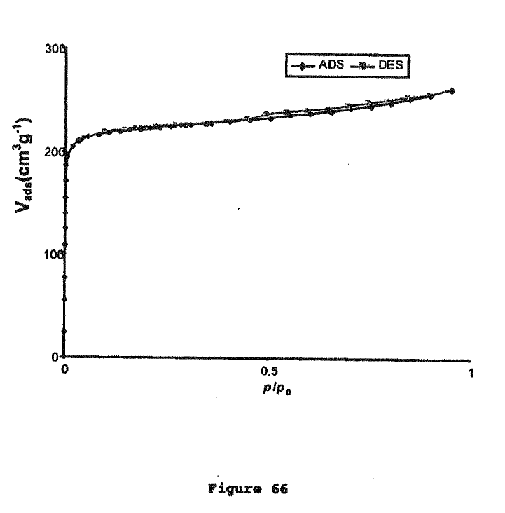

This compound exhibits a large surface area (Langmuir) (greater than 1200 m²/g) accessible to nitrogen at 77 K (FIG. 66).

z) iron 2,5-dihydroxoterephthalate or Fe₂ (₂OC—C₆H₂ (OH)₂—CO₂)(H₂O).xH₂O
Synthesis of the Solid Iron 2,5-dihydroxoterephthalate:

270 mg (1 mmol) of FeClO₃.6H₂O (Alfa Aesar, 98%) and 200 mg (1 mmol) of 2,5-dihydroxyterephthalic acid (obtained by hydrolysis of the corresponding diethyl ester, Aldrich, 97%) are dispersed in 5 ml of dimethylformamide (DMF, Aldrich, 99%). The mixture is left in a 23 ml Teflon body placed in a Paar metal bomb for 3 days at 150° C. with a temperature rise stage of 12 hours and a cooling stage of 24 hours. The solid is recovered by filtration.

The solid is then dried at 150° C. under a primary vacuum for 15 hours.

Characteristic Data for the Solid Iron 2,5-dihydroxyterephthalate:

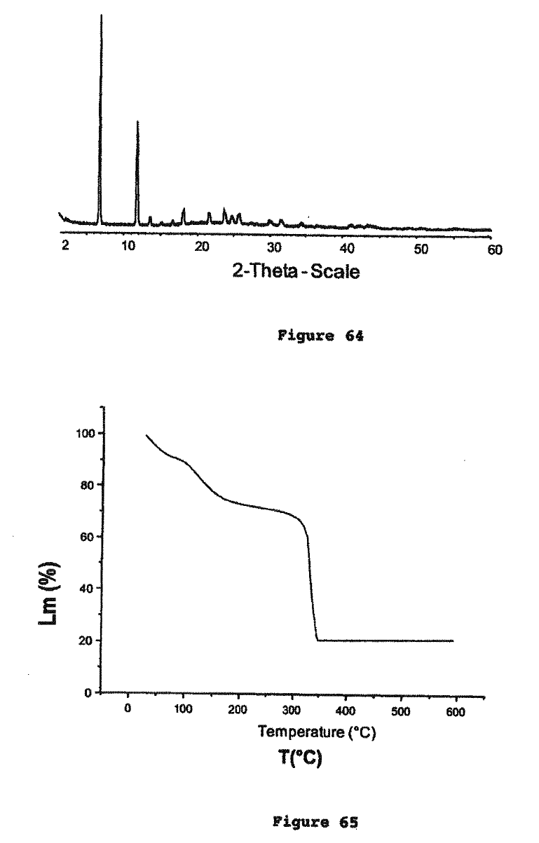

FIG. 64 represents the X-ray diffractogram of the crude solid iron 2,5-dihydroxyterephthalate. The phase, of trigonal symmetry, is an isotype of that published by Dietzel et al. with cobalt and nickel (space group R3) [61].

### Example 2

### Synthesis of Iron(III) Acetate

The iron(III) acetate, used in the examples below for synthesizing the MOF materials according to the invention, is synthesized according to the following protocol. For this synthesis, reference may be made to the publication by Dziobkowski et al., Inorg. Chem., 1982, 21, 671 [ref. 14].

6.72 g of iron metal powder (Riedel-de-Haen, 99%), 64 ml of deionized water and 33.6 ml of perchloric acid at 70% in water (Riedel-de-Haen) are mixed with magnetic stirring and heated at 50° C. for 3 hours. After the heating has been stopped, the solution is stirred for 12 hours. The residual iron metal is eliminated by settling out, followed by a change of vessel. 20.6 ml of hydrogen peroxide solution in water (sold by the company Alfa Aesar, 35%) are added dropwise with stirring, the whole mixture being kept in an ice bath at 0° C. 19.7 g of sodium acetate (Aldrich, 99%) are added to the blue solution with stirring, while keeping the solution at 0-5° C. The solution is left to evaporate for 3 days under a hood in a glass crystallizing dish (volume=0.5 l). Finally, the red crystals of iron acetate are recovered by filtration and washed very rapidly with ice-cold deionized water. The crystals are then dried in an air atmosphere.

### Example 3

### Synthesis of the Ligands

### a) Synthesis A: Synthesis of Chloroterephthalic Acid

6 g (0.043 mol) of chloroxylene (sold by the company Aldrich, >99%), 16 ml of nitric acid (sold by the company VWR, 70%) and 60 ml of distilled water are introduced into a 120 ml Teflon body. The latter is placed in a Paar metal bomb, and heated at 170° C. for 12 hours. The product is recovered by filtration, and then washed thoroughly with distilled water. A yield of 75% is obtained.

¹H NMR (300 MHz, d6-DMSO): δ (ppm): 7.86 (d, J=7.8 Hz), 7.93 (dd, J=7.8; 1.2 Hz), 7.96 (d, J=1.2 Hz).

### b) Synthesis B: synthesis of 3,5,3′,5′-tetramethylbiphenyl-4,4′-dicarboxylic acid

The reaction scheme for this synthesis is represented in FIG. 43.

### Stage 1:

10.2 g of tetramethylbenzidine (98%, Alfa Aesar) are suspended in 39 ml of concentrated hydrochloric acid (37%, sold by the company Aldrich) at 0° C. The diazotization is carried out by adding a solution of sodium nitrite (6 g in 50 ml of water). After stirring for 15 min at 0° C., a solution of potassium iodide (70 g in 200 ml of water) is added slowly to the resulting violet solution. Once the addition is complete, the mixture is stirred for 2 hours at ambient temperature. The resulting black suspension is filtered in order to recover a black precipitate, which is washed with water. The solid is suspended in dichloromethane (DCM, 98%, sold by the company SDS) and a saturated solution of sodium thiosulfate is added, causing decoloration. After stirring for 1 hour, the organic phase is separated by settling out and the aqueous phase is extracted with DCM. The organic phase is dried over sodium sulfate, and then evaporated so as to give the diiodo intermediate in the form of the grayish solid. Elution with pure pentane on a column of silica (sold by the company SDS) makes it possible to obtain the mixture of the monoiodo and diiodo compounds. The mixture of these compounds is used directly in the following stage.

### Stage 2:

7.2 g of the crude iodo compound are dissolved in 100 ml of tetrahydrofuran (THF, distilled over sodium). After cooling to −78° C., 35 ml of n-butyllithium in cyclohexane (2.5 M, sold by the company Aldrich) are added. The solution is allowed to return to ambient temperature, and a white suspension appears after 2 hours. It is again cooled to −78° C., and 12 ml of ethyl chloroformate are added. The mixture is left at ambient temperature, and a clear yellow solution is obtained after 1 hour. Partition between water and dichloromethane, followed by extraction with dichloromethane gives the crude diester. This product is purified by silica gel chromatography, elution being carried out with a 1/9 Et₂O/pentane mixture (front ratio: R_{f}=0.3). 6.3 g of diester are obtained in the form of a colorless solid (yield of 42% starting from benzidine).

Characterization of the diester obtained: ¹H NMR (300 MHz, CDCl₃): δ (ppm): 1.29 (t, J=7.2 Hz, 6H), 2.29 (s, 13H); 4.31 (q, J=7.2 Hz, 4H); 7.12 (s, 4H). ¹³(2 NMR (75 MHz, CDCl₃): δ (ppm): 14.3 (CH₃), 19.9 (CH₃), 61.0 (CH₂), 126.5 (CH), 133.2 (Cq), 135.5 (Cq), 141.4 (Cq), 169.8 (Cq).

### Stage 3:

Finally, the diester is saponified with 9.7 g of potassium hydroxide (sold by the company VWR) in 100 ml of 95% ethanol (sold by the company SDS), at reflux for 5 days. The solution is concentrated under vacuum and the product is dissolved in water. Concentrated hydrochloric acid is added to pH 1, and a white precipitate is formed. It is recovered by filtration, washed with water and dried. 5.3 g of diacid are thus obtained in the form of a white solid (quantitative yield).

### c) Synthesis C: synthesis of 3,3′-dimethylbiphenyl-4,4′-dicarboxylic acid

The reaction scheme for this synthesis is represented in FIG. 44.

The same procedure as that described for the synthesis B was used, starting with 12.1 g of dimethylbenzidine. At the end of stage 1, 18.4 g of 3,3′-dimethyl-4,4′-diiodobiphenyl are obtained (yield: 74%).

### Characterization of the Diiodo Compound Obtained:

¹H NMR (300 MHz, CDCl₃): δ (ppm): 2.54 (s, 6H), 7.10 (dd, J=2.2 and 8.1 Hz, 2H), 7.46 (d, J=2.2 Hz, 2H), 7.90 (d, J=8.1 Hz, 2H). ¹³C NMR (75 MHz, CDCl₃): (ppm): 28.3 (CH₃), 100.3 (Cq), 126.0 (CH), 128.3 (CH), 139.4 (CH), 140.4 (Cq), 141.9 (Cq).

At the end of stages 2 and 3, 6.9 g of 3,3′-dimethylbiphenyl-4,4′-dicarboxylic acid are obtained starting from the 18.4 g of diiodo compound.

### Characterization of the Compounds Obtained:

The diester obtained at the end of stage 2 and the diacid obtained at the end of stage 3 have spectroscopic signatures identical to those described in the literature (Shiotani Akinori, Z. Naturforsch. 1994, 49, 12, 1731-1736 [ref. 31]).

### d) Synthesis D: synthesis of 3,3f-dichloro-4,4′-azobenzenedicarboxylic acid

15 g of o-chlorobenzoic acid (sold by the company Aldrich, 98%) and 50 g of sodium hydroxide are placed in 225 ml of distilled water, and heated to 50° C. with stirring. 100 g of glucose (Aldrich, 96%) dissolved in 150 ml of water are added. The mixture is stirred for 15 minutes, and is then sparged with air for 3 hours at ambient temperature. The disodium salt is recovered by filtration, washed with ethanol, and then dissolved again in 120 ml of water. Hydrochloric acid (sold by the company Aldrich VWR, 37%) is added until a pH equal to 1 is obtained. The solid is recovered by filtration and dried under vacuum at 90° C.

### e) Synthesis E: 3,5,3′,5′-azobenzenetetracarboxylic acid

19 g of 5-nitroisophthalic acid (Aldrich, 98%) and 50 g of sodium hydroxide are placed in 250 ml of distilled water, and heated to 50° C. with stirring. A solution of 100 g of glucose (Aldrich, 96%) dissolved in 150 ml of water is added. The mixture is stirred for 15 minutes, and then sparged with air for 3 h at ambient temperature. The resulting disodium salt is recovered by filtration, and dissolved in 300 ml of water at ambient temperature. Hydrochloric acid (VWR, 37%) is added until a pH equal to 1 is obtained. The solid is recovered by filtration and dried under vacuum at 90° C.

### Example 4

### Synthesis of MOF Nanoparticles According to the Invention

a) MIL-89 nano

The synthesis of MIL-89 nano is carried out starting with iron acetate (1 mmol; synthesized according to the synthesis described in example 2) and muconic acid (1 mmol; Fluka, 97%) in 5 ml of ethanol (Riedel-de Haën, 99.8%) with the addition of 0.25 ml of 2M sodium hydroxide (Alfa Aesar, 98%) in an autoclave (Paar bomb) at 100° C. for 12 h. After cooling of the container, the product is recovered by centrifugation at 5000 rpm (revolutions per minute) for 10 minutes.

200 mg of the solid are then suspended in 100 ml of distilled water with stirring for 15 h in order to remove the solvent remaining in the pores. The solid is then recovered by centrifugation at 5000 rpm for 10 minutes.

The particle size measured by light scattering is 400 nm (nanometers).

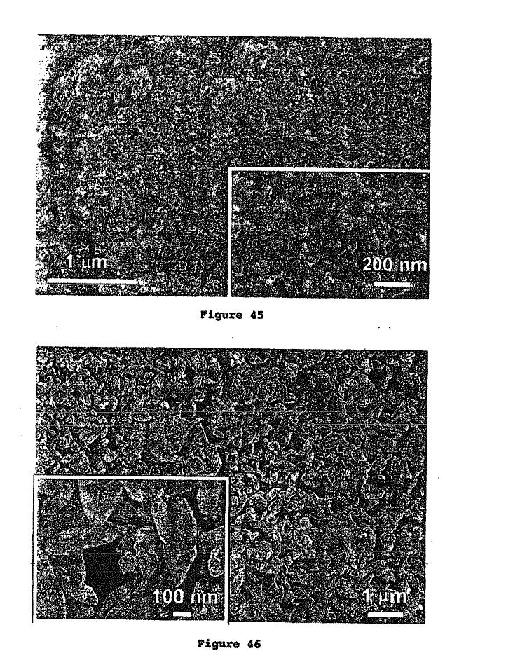

FIG. 45 represents an image obtained by scanning electron microscopy (SEM) of the solid MIL-89 nano.

The nanoparticles show a rounded and slightly elongated morphology, with a very homogeneous particle size of 50-100 nm (FIG. 45).

### b) MIL-88Anano

In order to obtain the material MIL-88Anano, FeCl₃.6H₂O (1 mmol; Alfa Aesar, 98%) and fumaric acid (1 mmol; Acros, 99%) are dispersed in 15 ml of ethanol (Riedel-de Haen, 99.8%). 1 ml of acetic acid (Aldrich, 99.7%) is then added to this solution. The solution is placed in a glass flask and heated at 65° C. for 2 hours. The solid is recovered by centrifugation at 5000 rpm for 10 minutes.

200 mg of the solid are suspended in 100 ml of distilled water with stirring for 15 h in order to remove the solvent remaining in the pores. The solid is then recovered by centrifugation at 5000 rpm for 10 minutes.

The particle size measured by light scattering is 250 nm.

The scanning electron microscopy (SEM) of the solid MIL-88Anano is represented in FIG. 46.

The SEM images (FIG. 46) show elongated particles with edges. There are two particle sizes: approximately 500 nm and 150 nm.

c) MIL-100 nano

The synthesis of MIL-100 nano is carried out by mixing FeCl₃.6H₂O (1 mmol; Alfa Aesar, 98%) and 1,3,5-benzenetricarboxylic acid (1,3,5-BTC; 1 mmol; Aldrich, 95%) in 3 ml of distilled water. The mixture is placed in a Paar bomb at 100° C. for 12 h. The product is recovered by centrifugation at 5000 rpm (10 minutes).

200 mg of the solid are suspended in 100 ml of distilled water with stirring at reflux for 3 h in order to remove the acid remaining in the pores. The solid is then recovered by centrifugation at 5000 rpm (10 minutes). The particle size measured by light scattering is 536 nm.

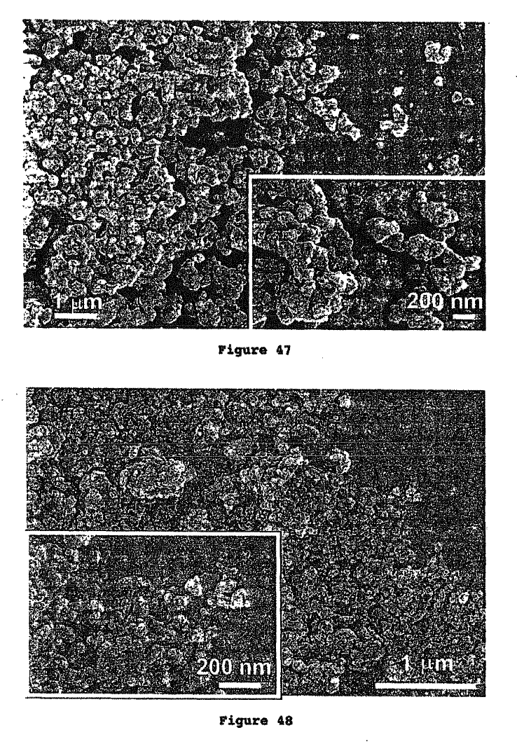

The scanning electron microscopy (SEM) of the solid MIL-100 nano is represented in FIG. 47.

The SEM images show strong aggregation of the particles (FIG. 47). The nanoparticles are rather spherical, but the size remains difficult to determine given the strong aggregation. A size of 40-600 nm can be estimated.

d) MIL-101 nano

In order to produce the solid MIL-101 nano, a solution of FeCl₃.6H₂O (1 mmol; Alfa Aesar, 98%) and 1,4-benzenedicarboxylic acid (1.5 mmol; 1,4-BDC, Aldrich, 98%) in 10 ml of dimethylformamide (Fluka, 98%) is placed in a Paar bomb and heated at 100° C. for 15 hours. The solid is recovered by centrifugation at 5000 rpm (10 minutes).

In order to remove the acid which remains in the pores, the product is heated at 200° C. under vacuum for 1 day. The product is kept under vacuum or an inert atmosphere given its low stability in air.

The particle size measured by light scattering is 310 nm.

### e) MIL-88Btnano

The solid MIL-88Btnano is synthesized from a solution of FeCl₃.6H₂O (1 mmol; Alfa Aesar, 98%) and 1,4-benzenetetramethyldicarboxylic acid (1 mmol; Chem Service) in 10 ml of dimethylformamide (Fluka, 98%) with 0.4 ml of 2M NaOH. This solution is placed in a Paar bomb and heated at 100° C. for 2 hours. The container is then cooled with cold water, and the product is recovered by centrifugation at 5000 rpm (10 minutes).

200 mg of the solid are suspended in 100 ml of distilled water with stirring for 15 h in order to remove the solvent remaining in the pores. The solid is then recovered by centrifugation at 5000 rpm (10 minutes).

The measurement of the particle size by light scattering shows two populations of nanoparticles of 50 and 140 nm.

The nanoparticles of the solid MIL-88Btnano have a spherical morphology with a size of approximately 50 nm. Only a minor fraction has a size of approximately 200 nm. Aggregates of small particles can also be observed therein.

The scanning electron microscopy (SEM) of the solid MIL-88Btnano is represented in FIG. 48.

### f) MIL-88Bnano

The solid MIL-88Bnano is synthesized from a solution of iron acetate (1 mmol; synthesized according to the synthesis described in example 2) and 1,4-benzenedicarboxylic acid (1 mmol; 1,4-BDC, Aldrich, 98%) in 5 ml of methanol (Aldrich, 99%). This solution is placed in a Paar bomb and heated at 100° C. for 2 hours. The container is then cooled with cold water, and the product is recovered by centrifugation at 5000 rpm (10 minutes).

200 mg of the solid are suspended in 100 ml of distilled water with stirring at reflux for 15 h in order to exchange the solvent which remains in the pores. The solid is then recovered by centrifugation at 5000 rpm (10 minutes).

The measurement of the particle size by light scattering shows a bimodal distribution of nanoparticles of 156 and 498 nm.

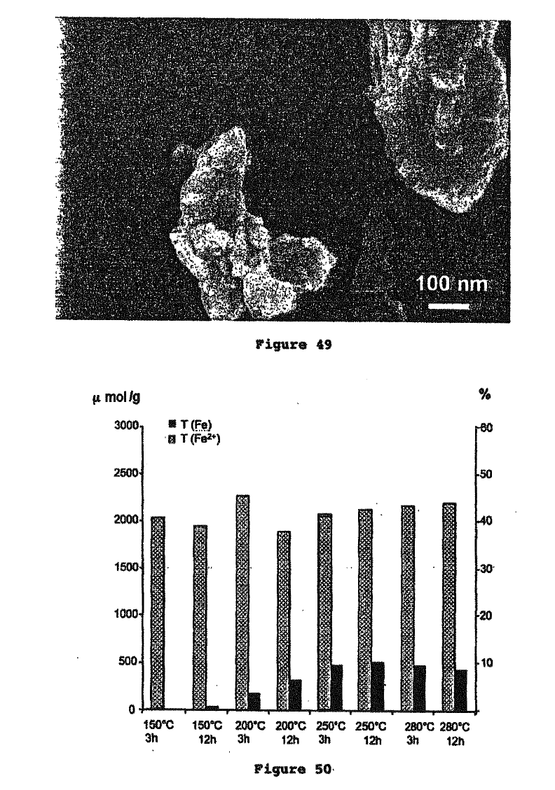

The scanning electron microscopy (SEM) of the solid MIL-88Bnano is represented in FIG. 49.

The morphology of the particles is very irregular, with a size of 300 nm.

The determination of the particle size by light scattering was carried out on a Malvern Zetasizer Nano series-Nano-ZS instrument (model Zen 3600; serial No. 500180; UK).

The scanning electron microscopy was carried out using a Topcon microscope (Akashi) EM 002B ultra-high resolution 200 kV.

The differences between the values obtained from the two techniques are explained, on the one hand, by the orange coloration of the iron carboxylate particles, the laser beam of the light scattering instrument being red, and, on the other hand, by particle aggregation phenomena.

### Example 5

### Synthesis of Iron(III) Carboxylates Surface-Modified with Chitosan

The surface modification of the nanoparticles with chitosan makes it possible to envision various routes of administration of the nanoparticles by virtue of the bioadhesion properties specific to this polymer.

In this example, the surface modification is carried out during the synthesis of the material MIL-88A.

### a) Preparation of the Surface-Modified Nanoparticles

7 mg of the surface-modifying agent, the modified chitosan, are added to a solution of FeCl₃.6H₂O (1 mmol, 270 mg; Alfa Aesar, 98%) and fumaric acid (1 mmol, 116 mg; Acros, 99%) in 5 ml of distilled water, in a 23 ml Teflon bomb. Two types of chitosan modified with alkyl chains (C12, lauryl) were used; one with modification of 2% of alkyl chains (Q25) and the other modified with 7% (Q100).

For complete dissolution of the chitosan, the solution is stirred for 45 minutes.

The Teflon bomb is placed in a hermetically sealed metal body and heated in an oven at 80° C. for 12 hours.

The solid obtained is recovered by centrifugation at 5000 rpm for 10 minutes and washed with distilled water and acetone.

### b) Analysis and Characterization

The size of the particles obtained is measured with a Malvern Zetasizer Nano series-Nano-ZS Z potential instrument; model Zen 3600; serial No. 500180; UK, observing a size of 2.64 and 0.91 microns for MIL88A-Q25 and MIL88A-Q100, respectively.

The X-ray diffraction (XRD) diagrams are collected with a Siemens D5000 X′Pert MDP diffractometer (λ_{Cu}, Kα₁, Kα₂) from 3 to 20° (20) with a step size of 0.04° and 2 s per step.

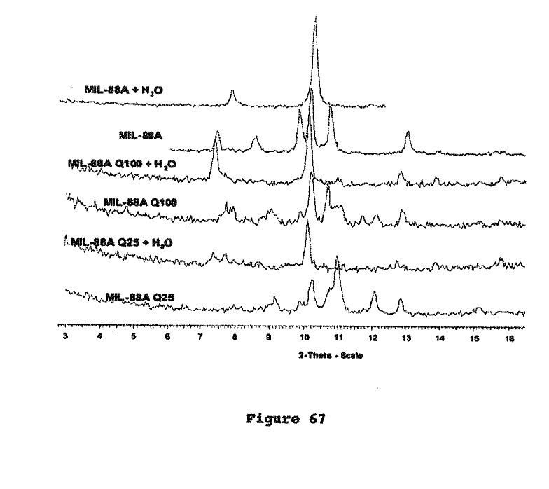

The XRD diagrams presented in FIG. 67 made it possible to verify that the phase obtained is indeed MIL-88A. The flexibility of the material is also verified by XRD by adding a drop of water to the solid.

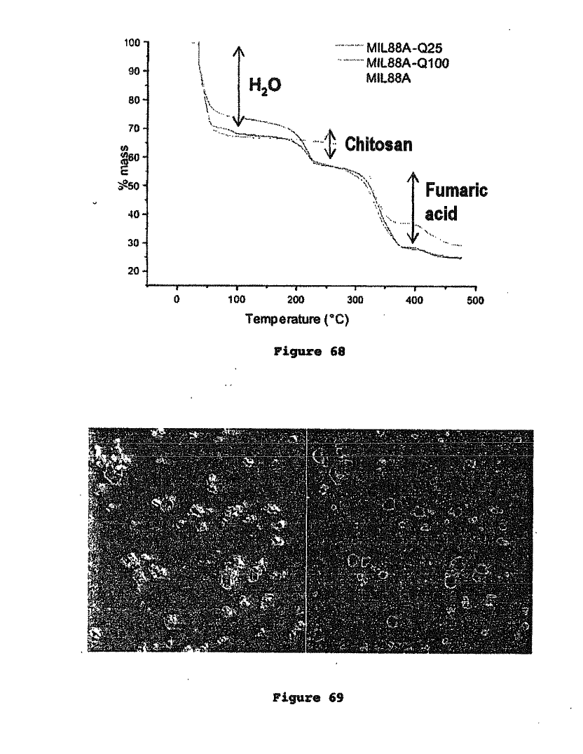

The amount of chitosan incorporated into the material is estimated by thermogravimetric analysis (TGA) presented in FIG. 68. The instrument used is a TA TGA2050 instrument from 25 to 500° C. with a heating ramp of 2° C./minute under a stream of oxygen (100 ml/min). In the materials, the amount of fumaric acid is indeed about 45% (relative to the dehydrated product). The materials MIL-88A-Q25 and MIL-88A-Q100 contain an amount of chitosan of about 16% and 22% (weight) relative to the dehydrated product, respectively.

### Example 6

### Synthesis of Iron(III) Carboxylates Surface-Modified with Fluorescein-Biotin Dextran

In this example, the dextran used is grafted firstly with fluorescein and, secondly, with biotin (Dex B FITC 10 000 g/mol, anionic, lysine-fixable, Molecular Probes, catalog D7178).

The characteristics of the dextran are as follows: dextran fluorescein and biotin, molecular weight 10 000 g/mol, anionic, capable of binding lysine (“mini-emerald”), batch 36031A, D7178, “Molecular probes”, In vitro detection technology, 1 mol fluorescein/mol, 2 mol biotin/mol.

### a) Preparation of Surface-Modified Nanoparticles

The iron 1,3,5-benzenetricarboxylate MIL-100 particles (particle diameter 1.79 microns) were washed with milliQ water.

Five milligrams of particles were dispersed in 0.5 ml of milliQ water. 0.5 ml of an aqueous solution of Dex FITC (5 mg/ml) was added to this suspension. They were incubated at ambient temperature for 24 h, and then recovered by centrifugation (3800 rpm, 10 minutes). The supernatant was removed and then the pellet (particles) was resuspended in 0.5 ml of milliQ water. After a further centrifugation, the particles thus washed free of the excess Dex B FITC were placed on a cover slip in order to be observed under a confocal microscope (excitation 488 nm, emission 515 nm).

### b) Analysis and Characterization

The fluorescein allows detection of the particles using a laser scanning confocal microscope, whereas the biotin, which is hydrophobic, allows:

1. anchoring in the core of the particles,
2. functionalization with biotinylated ligands.

FIG. 69 shows the optical sections thus obtained. A halo is distinguished around the particles, indicating the presence of dextran (sole fluorescent compound) only at the surface. This is because the long polymer chains were not able to penetrate into the core of the particles.

This surface modification method has the advantage of not disturbing the core of the particles (containing active ingredients) and of being performed post-synthesis, and thus offering a variety of possible coverings.

### Example 7

### Synthesis of Iron(III) Carboxylates Surface-Modified with Polyethylene Glycol (PEG)

In order to minimize the toxicity of busulfan in the liver, the nanoparticles need to be prevented from being directed toward the liver; the best method consists in surface-grafting the hybrid nanoparticles with hydrophilic chains of the polyethylene glycol (PEG) type, so as to reduce their accumulation in this organ. We envision a full study of the in vitro degradation of the particles covered or not covered with PEG, in different media.

The PEG chains may have various end groups so as to graft to the surface of the materials. Thus, the interaction of PEG with the particle surface may be modified using various types of PEG.
PEG-NH₂ (alpha-t-butyloxycarbonylamino-omega-amino poly(ethylene glycol) (PEG; Boc-NH-PEG-NH2, 5000 MW, Iris Biotech);
PEG-COOH (poly(ethylene glycol) carboxylic acid, Iris Biotech);
PEG-PO₄ synthesized in the laboratory according to the following process:

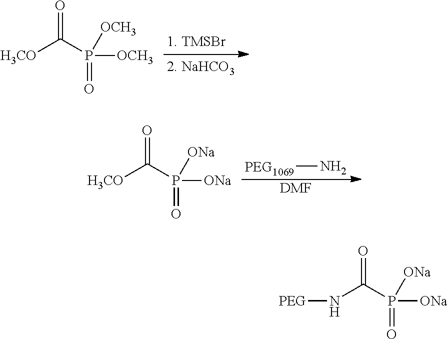

The phosphonate group is attached to the PEG-NH₂ via a condensation of amide with an ester bound to a phosphonate group. The sodium salt of the phosphonate was used. Next, the coupling was carried out starting with trimethyl phosphonoformate [CAS 31142-23-1] according to the procedure described by Robert A. Moss, Hugo Morales-Rojas, Saketh Vijayaraghavan and Jingzhi Tian, J. Am, Chem. Soc., 2004, 126 (35), 10923-10936 [52].

Dissolution of the PEG-NH₂ (87.6 mg, M=5400, Iris Biotech GmbH, PEG 1069) in 2 ml of DMF (Fluka, 97%) with an excess of disodium methylphosphonoformate (50 mg, M=183.99) was heated at 100° C. for 15 h with stirring. Next, the solvent was removed under vacuum and the residue was suspended in absolute ethanol. The excess phosphonoformate is insoluble, and may thus be removed by filtration. The filtrate is concentrated so as to give the product (85 mg). ³¹P NMR, (D₂O), δ=1.3 ppm.

The modification with polyethylene glycol may be carried out during or after the synthesis as indicated below.

a) Surface Modification with PEG-COOH During Synthesis of the Nanoparticles

The syntheses of the MOFs are performed directly in the presence of monoethoxy PEG monoacid (MeO-PEG-COOH) of general formula CH₃—O—(CH₂—CH₂—O)ₙ—CH₂—CH₂—COOH (Sigma, molar mass 5000 g/mol).

The monomethoxy PEG monoacid is introduced at 3, 8.5 or 13% by mass relative to the total weight of solid used in the synthesis.

### Preparation Process:

Iron acetate (1 mmol; synthesized according to synthesis A described in example 2) and muconic acid (1 mmol; Fluka, 97%) are mixed in 10 ml of methanol (Aldrich, 99.9%). The whole is introduced into a 23 ml Teflon body. The PEG monoacid is then introduced in an amount of 3, 8.5 or 13% by mass relative to the total weight of solid. 0.35 ml of 2 M sodium hydroxide is optionally added. The solution is stirred for 20 minutes.

The Teflon bomb is placed in a hermetically sealed metal body and heated in an oven at 100° C. for 12 hours.

The solid obtained is recovered by centrifugation at 5000 rpm for 10 minutes and washed with distilled water and acetone.

The assaying of the PEG in the iron carboxylates is carried out as follows: the particles are totally degraded in acidic medium (5M HCl) so as to release the associated PEG. After neutralization of the solutions obtained (at pH=7) and destruction of the nanoparticles with sodium hydroxide, the PEG was assayed by UV spectrophotometry (at a wavelength of 500 nm), according to the method described in B. Baleux et al. C. R. Acad. Science Paris, series C, 274 (1972) pages 1617-1620 [53]. The main results are collated in the following table.

TABLE 10
Modification of the material MIL88A with PEG
5000 g/mol

| Addition | Mass % of PEG | Mass % of PEG | Nanoparticle |
|---|---|---|---|
| of aqueous | introduced at the | in the | diameter (nm) |
| NaOH | start of | nanoparticle | (measured by light |
| solution | synthesis | composition | scattering) |
| — | 3 | 3.8 | 570 |
| yes | 3 | 4.8 | 230 |
| — | 8.5 | 13.4 | 590 |
| yes | 8.5 | 13 | 230 |
| — | 13 | 18.5 | 565 |
| yes | 13 | 18 | 310 |

It can be noted that:
the addition of sodium hydroxide makes it possible to reduce the size of the nanoparticles;
the mass % of PEG in the nanoparticles is greater than the mass % of PEG introduced at the start of synthesis;
it is, remarkably, possible to obtain particles of approximately 230 nm containing 13% by weight of PEG, which is advantageous for medical applications (“stealth”).

Specifically, the “stealth” nanoparticles described in the literature generally contain less than 10% by mass of PEG, as described in R. Gref et al. Colloids and Surfaces B: Biointerfaces, volume 18, issues 3-4, October 2000, pages 301-313 [54].

b) Surface Modification of MIL-100 Nanoparticles with PEG after Synthesis of Said Nanoparticles

The MIL-100 nanoparticles are synthesized by the microwave process (CEM microwave) starting with a solution of 9.7 g of iron nitrate hexahydrate (Aldrich, 97%), 3.38 g of 1,3,5-benzenetricarboxylic acid (1,3,5-BTC, Aldrich, 99%) and 40 g of distilled water at 180° C. for 30 min (power 600 W). The particle size measured by light scattering is 400 nm.

The pegylated MIL-100 nanoparticles modified with polyethylene glycol are obtained by surface modification of the particles mentioned previously. 30 mg of MIL-100 are suspended in 3 ml of an aqueous solution of 10 mg of amino-terminal polyethylene glycol (PEG-NH2 5000 g/mol, Aldrich, 97%) at 30° C. for 3 hours with stirring. These nanoparticles are recovered by centrifugation (10 000 rpm/10 min) and washed with distilled water.

The amount of surface PEG is determined by the method of Baleux and Champertier, based on the formation of a complex stained with iodine-iodide on the PEG, which is selectively measured by spectrophotometry at 500 nm.

The amount of PEG is 19% by mass and the particle size after modification with polyethylene glycol increases to 800 nm. On the other hand, the observation of nanoparticles modified and not modified with PEG, by scanning electron microscopy (SEM), shows nanoparticles of 150 nm in both cases. This difference may be due to particulate aggregation phenomena.

### Example 8

### Synthesis Via the Ultrasonication Process of Iron(III) Carboxylates Surface-Modified with Polyethylene Glycol (PEG)

The synthesis, by the ultrasonication process, of nanoparticles of solid MIL-88A surface-modified with PEG was carried out at various reaction times (30, 60, 90 and 120 minutes).

In the examples which follow, two procedures were carried out:

a) in the first procedure, the PEG is added 15 minutes before the end of the synthesis,
b) in the second procedure, the PEG is added at the start of the synthesis (t=0 min).

For each of the syntheses below, aqueous solutions of iron(III) chloride (2.7 mg/ml; FeCl₃.6H₂O sold by Acros, 97%) and of fumaric acid (1.16 mg/ml; C₄H₄O₄ sold by Acros, 99%) are prepared. The two solid reactants are weighed and dissolved separately in water in the proportions given in the examples below. The fumaric acid solution is brought to 70° C. with stirring for 120 min in order to solubilize the product. The iron chloride is stirred with a magnetic stirrer for 30 min.

### a) Synthesis No. 1:

In total, 8 flasks are prepared. 5 ml of iron(III) chloride solution (2.7 mg/ml) and 5 ml of fumaric acid solution (1.16 mg/ml) are added to each of the eight 20 ml flasks:
4 flasks serve as a control in which the reactions are carried out for the 4 synthesis times: 30, 60, 90 and 120 min,
in the other 4 flasks, 5 mg of PEG are added 15 min before the end of each of the syntheses lasting 30, 60, 90 and 120 min (the end of a synthesis corresponds to the removal from the ultrasonication bath).

The 8 flasks are placed at the same time in a sonication bath at 0° C., for the corresponding times t (30, 60, 90 and 120 min).

After the synthesis, a volume of 0.1 ml of solution is taken from each flask in order to determine the particle size by light scattering using a Dynamic Light Scattering instrument (DLS, Nanosizer). The rest of the solution is then centrifuged at 10 000 rpm at 0° C. for 15 min in order to separate the supernatant from the solid formed. The supernatant is removed using a Pasteur pipette and the pellet recovered is placed under a fume cupboard at ambient temperature (approximately 20° C.)

### Equipment Used:

Sonication bath: Labo-moderne TK 52H serial No.: 164046192 Sonoclean
Centrifuge: Jouan MR 1812
Nanosizer: Coulter N4 PLUS USA; Malvern.

### b) Synthesis No. 2:

In total, 8 flasks are prepared. 5 ml of iron(III) chloride solution (2.7 mg/ml) and 5 ml of fumaric acid solution (1.16 mg/ml) are added to each of the eight 20 ml flasks:
4 flasks serve as a control in which the reactions are carried out for the four synthesis times: 30, 60, 90 and 120 min,
in the other 4 flasks, 5 mg of PEG are added at the start of each of the syntheses, lasting 30, 60, 90 and 120 min.

The 8 flasks are placed at the same time in a sonication bath at 0° C., for the corresponding times t (30, 60, 90 and 120 min).

After the synthesis, a volume of 0.1 ml of solution is taken from each flask so as to determine the particle size by light scattering using a Dynamic Light Scattering instrument (DLS, Nanosizer). The rest of the solution is then centrifuged at 10 000 rpm at 0° C. for 15 min so as to separate the supernatant from the solid formed. The supernatant is removed using a Pasteur pipette and the pellet recovered is placed in a fume cupboard at ambient temperature (approximately 20° C.)

### Equipment Used:

Sonication bath: Labo-moderne TK 52H serial No.: 164046192 Sonoclean
Centrifuge: Jouan MR 1812
Nanosizer: Coulter N4 PLUS USA; Malvern.

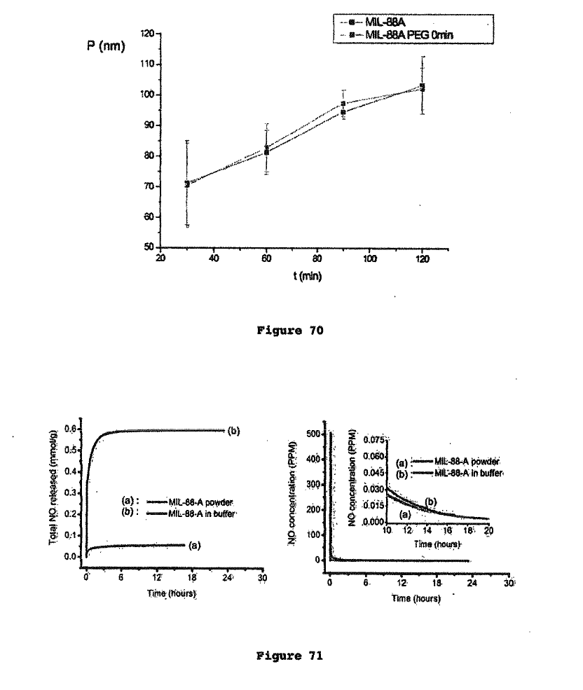

The change in particle size (P in nm) as a function of time (t in min) is represented in FIG. 70. This figure shows that there is no significant variation after the addition of the PEG at the initial synthesis time.

Whether in the presence or absence of PEG at the initial synthesis time, it is possible to observe by XRD a shoulder at 11° C., characteristic of the MIL-88A phase, which appears to increase in intensity with the synthesis time.

### c) Conclusion of the Study:

The aim of this study was to optimize the particle size, which must be less than 200 nm so as to be able to make the particles compatible with intravenous administration. The results obtained are satisfactory since the particle diameters obtained are less than 200 nm (with verification of the crystal structures of MIL-88A type in most solids). Furthermore, even though the yields are less then those obtained by the solvothermal process or the microwave process, they may be considered to be acceptable (table below).

TABLE 11
Synthesis yields via the ultrasonication
process

Yield (%)

| Time | | | | PEG t = end |
|---|---|---|---|---|
| (min) | Control | AcH | PEG t = 0 | −15 min |
| 30 | 24 | 13.4 | 31.4 | 20.1 |
| 60 | 27.2 | 15 | 29.4 | not measured |
| 90 | 35.6 | 14 | 24 | 28.3 |
| 120 | 35.1 | 19.1 | 32 | 41.2 |

It is possible to observe that the particle size increases as a function of the synthesis time.

Similarly, the modification with PEG at t=0 min results in smaller particle diameters than the modification with PEG at t=end−15 min, probably due to the fact that the crystal growth is stopped earlier.

### Example 9

### Synthesis of MOF Solids Surface-Modified with Polyethylene Glycol (PEG) and Folic Acid (FA)

### Folic Acid:

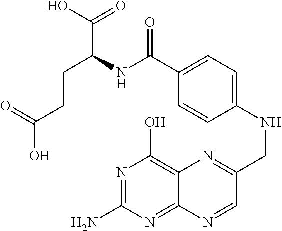

a) Synthesis No. 1: Surface Modification after Synthesis of the Nanoparticles
Surface Modification with PEG:

100 mg of MIL100, MIL88, MIL53 or MIL101 nanoparticles (dehydrated beforehand at 100° C./overnight) are dispersed with sonication in 100 ml of solution containing 17.9 mM of 2-(methoxy(polyethyleneoxy)propyl)trimethoxysilane in anhydrous toluene. The mixture is subjected to ultrasound at 60° C. for 4 h, under a stream of inert gas (nitrogen). The resulting colloidal suspension, containing the nanoparticles surface-modified with PEG, is washed twice with ethanol and twice in a 20 mM sodium citrate solution (pH 8.0) and finally resuspended in water.

Surface Modification with PEG and FA:

The FA was attached to the nanoparticles by means of a difunctional spacer, silane-PEG-trifluoroethyl ester (TFEE) synthesized according to a method described in the literature by Kohler N. et al., J Am Chem Soc 2004; 126; 7206-7211 [55].

100 mg of nanoparticles are covered with PEG-TFEE according to the same method as described above, using silane-PEG-TFEE in place of 2-methoxy(polyethyleneoxy)propyltrimethoxysilane.

The resulting nanoparticles, covered with PEG-TFEE, are washed twice and then resuspended in 100 ml of dry toluene. A primary amine was grafted onto the end groups of the PEG chains by adding 1 ml of ethylenediamine (Sigma) to the nanoparticles maintained under a stream of nitrogen. The mixture was ultrasonicated (4 h, 60° C.). The resulting nanoparticles, covered with the amine, were washed three times with ethanol and three times with dimethyl sulfoxide (DMSO). The nanoparticles were finally resuspended in 50 ml of anhydrous DMSO. The FA was coupled to the amine end groups of the PEG chains by adding 50 ml of FA solution (23 mM FA in DMSO) with equimolar amounts of dicyclohexylcarbodiimide (DCC) (Sigma) and 10 μl of pyridine. The mixture was protected from light and left to react overnight with two-dimensional stirring (180 rpm). The nanoparticles conjugated with PEGH and FA (NP-PEG-FA) were washed twice with ethanol and twice with a 20 mM sodium citrate solution (pH 8.0) and finally resuspended in this same sodium citrate solution.

### b) Synthesis No. 2: Surface Modification During the Synthesis of the Nanoparticles

The surface modification of the MOF solids can also be carried out during the synthesis.

In the example which follows, the surface modification is carried out with chitosan grafted beforehand with folic acid (FA).

An example of synthesis of chitosan grafted with folic acid via a PEG spacer is described in the publication by Peggy Chan et al., Biomaterials, volume 28, issue 3, 2007, pp 540-549 [56].

The following reactants were used for carrying out this example:
chitosan (molar mass Mn of 255 kDa, viscosity: 200-800 cps in 1% acetic acid, sold by the company Sigma-Aldrich),
N-hydroxylsuccinimide-PEG-maleimide (NHS-PEG-MAL, Mn 3400 Da, sold by the company Nektar, NOF Corporation, Tokyo, Japan), the succinimidyl ester of monomethoxy-PEG (mPEG-SPA, Mn 5000 Da, sold by the company Nektar, NOF Corporation, Tokyo, Japan).

The chitosan is deacetylated beforehand to obtain a degree of acetylation of 82% (determined by ¹H-NMR) according to the process described by Wang LS (Thesis, National University of Singapore, Singapore, 2001).

100 mg of chitosan were dissolved in 50 ml of acetic acid solution (20%). The pH of the solution was adjusted to 6 by adding sodium hydroxide and the mPEG-SPA was introduced in the reaction mixture. The mixture was left to react for 24 h at ambient temperature with stirring. The product obtained was dialyzed for 24 h against deionized water, using a membrane with a cutoff threshold of 12 000 Da (Spectrum Laboratories, USA) and, finally, lyophilized.

To synthesis the chitosan grafted with PEG and FA, the N-hydroxysuccinimide ester of FA was prepared according to the method described by J. H. Van Steenis et al., J Control Release 87 (2003), pp. 167-176 [57].

Briefly, 1 g of FA was added to a mixture of anhydrous DMSO (40 ml) and triethylamine (TEA, 0.5 ml). The mixture was stirred in the dark overnight, under anhydrous conditions. The other reactants, dicyclohexylcarbodiimide (DCC, 0.5 g) and N-hydroxysuccinimide (NHS, 0.52 g), were added and the mixture was left to react for 18 h in the dark under anhydrous conditions. The precipitated by-product, dicyclohexylurea (DCU), was removed by filtration. The DMSO and the TFA were evaporated off under vacuum. The reaction product was dried under vacuum, and then dissolved in 1.5 ml of a 2/1 (v/v) mixture of DMSO and TEA. An equimolar amount of 2-aminoethanethiol (Wako) was added and the reaction was allowed to continue overnight under anhydrous conditions. Thus, a thiol group could be introduced onto the folic acid, and the resulting product is known as FA-SH.

100 mg of chitosan are dissolved in 50 ml of acetic acid solution (20%). The pH of the solution is adjusted to 6 by adding sodium hydroxide, and 100 mg of NHS-PEG-Mal are introduced into the reaction mixture.

The mixture is left to react for 3 h at ambient temperature (approximately 20° C.) with stirring, and then the pH is adjusted to 7. The mixture is left to react overnight, under anhydrous conditions. The FA-SH synthesized as previously was added gradually with stirring and the pH was adjusted to 6.5-7.5 with sodium hydroxide.

The conjugate obtained, known as FA-PEG chi, bears FA coupled to chitosan via a PEG spacer arm, which is an advantage for reaching the folic acid receptor (as described in the literature, see, for example: A. Gabizon, H. Shmeeda, A. T. Horowitz and S. Zalipsky, Tumor cell targeting of liposome-entrapped drugs with phospholipids-anchored folic acid-PEG conjugates, Adv Drug Deliv Rev 56 (2004), pp. 1177-1192 [58]).

The degree of substitution can be adjusted by varying the PEG/chitosan mass ratio used in the reaction. This polymer was dialyzed for 48 h against deionized water using a membrane with a cutoff threshold of 12 000 Da (Spectrum Laboratories, USA) and finally lyophilized.

### c) Synthesis No. 3:

The hybrid solids can be surface-modified by adsorption of polysaccharides such as biotin-grafted dextran.

It is thus possible to envision adsorbing, in place of biotin-grafted dextran, chitosan grafted with folic acid (synthesized as described in the publication cited above) and, optionally, if necessary, also grafted with other hydrophobic compounds such as cholesterol or aliphatic chain units, so as to provide better adhesion at the surface of the nanoparticles.

Surface functionalization may also be carried out via adsorption of other FA-grafted polysaccharides.

### d) Synthesis No. 4:

The hybrid solids can be surface-modified with PEG during their synthesis. The monomethoxy PEG monoacid used in this synthesis is substituted with PEG monoacid comprising a reactive function blocked at the chain end, for instance the commercial product:

Boc-PEG-carbonateNHS, MW 5000, Boc=tert-butoxycarbonyl (reference Sunbright® BO-050TS, NOF Corporation).

After reaction, as indicated in the example, mixtures of MeO-PEG-COOH and Boc-PEG-carbonateNHS (mass ratios 1:0.05 to 1:0.5) are used in place of MeO-PEG-COOH. The deprotection will be carried out by adding trifluoroacetic acid (TFA).

### Procedure:

0.6 ml of TFA is added to a suspension of 300 mg of nanoparticles in 2 ml of water. The mixture is left to react for 1 h at ambient temperature (approximately 20° C.) with magnetic stirring. The particles are isolated by centrifugation and washed three times with distilled water.

The reactive groups at the surface are functionalized with ligands such as FA, for example as in synthesis No. 1 indicated in paragraph a).

### e) Characterization of the Nanoparticles:

The amount of folic acid actually coupled to the nanoparticles may be determined after degrading them in an acidic medium, neutralizing to pH 7 and then redissolving in a suitable solvent, such as dichloromethane, DMSO or a mixture of these two solvents. The folic acid may then be quantified by measuring the UV absorbence (at 358 nm, the molar extinction coefficient E of folic acid is 15.76 M⁻¹·cm⁻¹).

In order to verify that the folic acid is indeed at the surface of the nanoparticles, the surface plasmon resonance technique (BIAcore) is used. The folate binding protein is immobilized at the surface of the detector, on a thin film of activated dextran (conventional procedure recommended by the manufacturer BIAcore). The amount of nanoparticles actually attached to this support is evaluated relative to that of nanoparticles not covered with folic acid.

### Example 10

### Synthesis of MOF Materials Based on Bioactive Ligands

The use of ligands with a biological activity is of value for:
the release of active compound by degradation of the MOF material;
the encapsulation of other active molecules for combined therapies.

Tests for antimicrobial activity, and also for degradation in physiological media and activity on cells will be carried out on porous iron carboxylates having a flexible structure of MIL-88 type, using 4,4′-azobenzenedicarboxylic acid and 3,3′-dichloro-4,4′-azobenzenedicarboxylic acid, inter alia.

In the syntheses which follow, various bioactive molecules are used to prepare the MOF materials of the present invention, and in particular: azobenzene, azelaic acid and nicotinic acid.

Azoebenzene (AzBz), of formula C₆H₅—N═N—C₆H₅, can be incorporated into polymer matrices as a stabilizer. In addition, the rigid structure of azo molecules allows them to behave as liquid-crystal mesogens in many materials. Moreover, azobenzene can be photoisomerized (cis or trans isomer), hence its use for photo-modulating the affinity of a ligand (for example, a medicament) for a protein. Specifically, azobenzene can act as a photoswitch between a ligand and a protein by allowing or preventing protein-medicament binding according to the cis or trans isomer of azobenzene (one end of the azobenzene can be substituted, for example, with a group that binds to the target protein, while the other end is connected to a ligand (medicament) for the protein).

Azelaic acid (HO₂C—(CH₂)₇—CO₂H) is a saturated dicarboxylic acid which has antibacterial, keratolytic and comedolytic properties. It is used in particular in the treatment of acne and rosacea.

Nicotinic acid (C₅H₄N—CH₂H) is one of the two forms of vitamin B3, with nicotinamide. Vitamin B3 is in particular necessary for the metabolism of carbohydrates, fats and proteins.

a) MIL-88G (AzBz) (Fe) or Fe₃O [C₁₂H₈N₂—(CO₂)₂]₃.X.nH₂O (X═F, Cl, OH)

118 mg of Fe(ClO₄)₃.xH₂O (0.33 mmol, Aldrich, 99%) and 90 mg (0.33 mmol) of 4,4′-azobenzenedicarboxylic acid (synthesized according to the method described by Ameerunisha et al., J. Chem. Soc. Perkin Trans. 2, 1679, 1995 [59]) are dispersed in 15 ml of DMF (Fluka, 98%). The whole is left in a 23 ml Teflon body placed in a Paar metal bomb for 3 days at 150° C. The solid is recovered by filtration.

200 mg of the solid are suspended in 10 ml of DMF with stirring at ambient temperature for 2 h in order to exchange the acid remaining in the pores. The solid is then recovered by filtration and then calcined at 150° C. under vacuum for 15 hours in order to remove the DMF remaining in the pores.

The particle size measured by light scattering is >1 micron.

b) MIL-88G-2C1(AzBz-2C1) (Fe) or Fe₃O[C₁₂H₆N₂Cl₂—(CO₂)₂]₃.X.nH₂O (X═F, Cl, OH)

177 mg of Fe(ClO₄)₃.xH₂O (0.5 mmol, Aldrich, 99%) and 169 mg (0.5 mmol) of dichloro-4,4′-azobenzenedicarboxylic acid (prepared according to synthesis D described in example 3) are dispersed in 15 ml of DMF (Fluka, 98%). The whole is left in a 23 ml Teflon body placed in a Paar metal bomb for 12 h at 150° C. The solid is recovered by filtration.

200 mg of the solid are suspended in 10 ml of DMF with stirring at ambient temperature for 2 h in order to exchange the acid remaining in the pores. The solid is then recovered by filtration and then calcined at 150° C. under vacuum for 15 h in order to remove the DMF remaining in the pores.

The particle size measured by light scattering is >1 micron.

c) Iron azobenzene-3,3′,5,5′-tetracarboxylate 1

118 mg of Fe(ClO₄)₃.xH₂O (0.3 mmol, Aldrich, 99%) and 119 mg (0.3 mmol) of 3,3′,5,5′-azobenzenetetracarboxylic acid (prepared according to the synthesis E described in example 3) are dispersed in 15 ml of DMF (Fluka, 98%) with 0.1 ml of 5M HF (SDS, 50%). The whole is left in a 23 ml Teflon body placed in a Paar metal bomb for 3 days at 150° C. The solid is recovered by filtration and washed with acetone.

The solid obtained has a rigid cubic structure.

The particle size measured by light scattering is >1 micron.

d) Iron azobenzene-3,3′,5,5′-tetracarboxylate 2

118 mg of Ee(ClO₄)₃.xH₂O (0.3 mmol, Aldrich, 99%) and 119 mg (0.3 mmol) of 3,3′,5,5′-azobenzenetetracarboxylic acid (prepared according to synthesis E described in example 3) are dispersed in 15 ml of distilled water with 0.1 ml of 5M HF (SDS, 50%). The whole is left in a 23 ml Teflon body placed in a Paar metal bomb for 3 days at 150° C. The solid is recovered by filtration and washed with acetone.

The particle size measured by light scattering is 498 nm, with a second minor population of 1100 nm.

### e) Iron Azelate 1

270 mg of FeCl₃.6H₂O (1 mmol, Aldrich, 99%) and 188 mg (1 mmol) of azelaic acid (Aldrich, 99%) are dispersed in 5 ml of distilled water. The whole is left in a 23 ml Teflon body placed in a Paar metal bomb for 3 days at 100° C. The solid is recovered by filtration and washed with acetone.

200 mg of the solid are suspended in 50 ml of absolute ethanol with stirring for 5 h in order to activate it. The solid is recovered by filtration.

The particle size measured by light scattering is >1 micron (1500 nm).

### f) Iron Nicotinate 1

The synthesis conditions in water are as follows:

135 mg of FeCl₃.6H₂O (1 mmol, Aldrich, 99%) and 62 mg (1 mmol) of nicotinic acid (Aldrich, 99%) are dispersed in 5 ml of distilled water with 0.1 ml of 2M NaOH. The whole is left in a 23 ml Teflon body placed in a Paar metal bomb for 16 hours at 100° C. The solid is recovered by filtration and washed with acetone.

The synthesis conditions in DMF are as follows:

135 mg of FeCl₃.6H₂O (1 mmol, Aldrich, 99%) and 62 mg (1 mmol) of nicotinic acid (Aldrich, 99%) are dispersed in 5 ml of DMF (Fluka, 98%). The whole is left in a 23 ml Teflon body placed in a Paar metal bomb for 16 h at 100° C. The solid is recovered by filtration and washed with acetone.

The monodisperse particle size (PDI=0.241) measured by light scattering is 662 nm.

### g) Iron Nicotinate 2

The synthesis conditions for the iron nicotinate 2 are as follows:

71 mg of iron(III) acetate (0.12 mmol, according to the process described previously) and 73.8 mg (0.6 mmol) of nicotinic acid (Aldrich, 99%) are dispersed in 5 ml of DMF (Fluka, 98%). The whole is left in a 23 ml Teflon body placed in a Paar metal bomb for 24 hours at 140° C. The solid is recovered by filtration and washed with acetone.

The crystallographic data for this phase are in FIG. 89:

Space group P 21/n
a=16.422899 b=21.423-401 c=11.048300
beta=91.806999

### Example 11

### Determination of the Iron Content in the Solid MIL-100(Fe)

### Activation:

In order to empty the pores of the material (residual solvents, acids) and to free the metal coordination sites, the material MIL-100(Fe) was activated by heating at 150° C. under a primary vacuum for 15 hours. The resulting solid possesses only iron in oxidation state +III.

### Fe³⁺/Fe²⁺Reduction:

The partial reduction of the material MIL-100(Fe) was carried out by heating at 250° C. under a primary vacuum for 15 hours. Infrared spectroscopy made it possible to quantify the relative iron(II)/iron(III) content at around 20/80% (FIG. 50).

FIG. 50 represents the amount of coordinatively unsaturated iron sites present in the activated solid MIL-100(Fe) as a function of the heat treatment carried out. The solid MIL-100(Fe) is activated under a residual vacuum (approximately 10⁻⁵ Torr) at various temperatures and for various periods of time. T(Fe) represents the content of coordinatively unsaturated iron sites and T(Fe²⁺) represents the content of coordinatively unsaturated Fe²⁺sites (in mmol of unsaturated sites per gram of activated solid or as % of unsaturated iron sites).

The amounts of unsaturated iron sites are determined by CO adsorption at 100 K, followed by infrared spectroscopy. The uncertainty with respect to the values is estimated at +/−10%.

### Example 12

### Demonstration of the Flexibility of the Solids

The category of flexible hybrid solids based on trimers of trivalent transition metals is known as MIL-88. These compounds are typically constructed from octahedral iron trimers, i.e. three iron atoms connected by a central oxygen atom and by six carboxylate functions connecting the iron atoms in pairs; a terminal water molecule, coordinated to each iron atom, then completes the octahedral coordinance of the metal. These trimers are then linked together by aliphatic or aromatic dicarboxylic acids so as to form the solids MIL-88A, B, C, D and MIL-89 (-A for fumaric acid, -B for terephthalic acid, -C for 2,6-naphthalenedicarboxylic acid, -D for 4,4′-biphenyldicarboxylic acid and MIL-89 for trans, trans-muconic acid), as described in the document by Serre et al., Angew. Chem. Int. Ed. 2004, 43, 6286 [17]. Other analogs with other dicarboxylic acids have also been synthesized and are known as MIL-88E, F, G, etc.

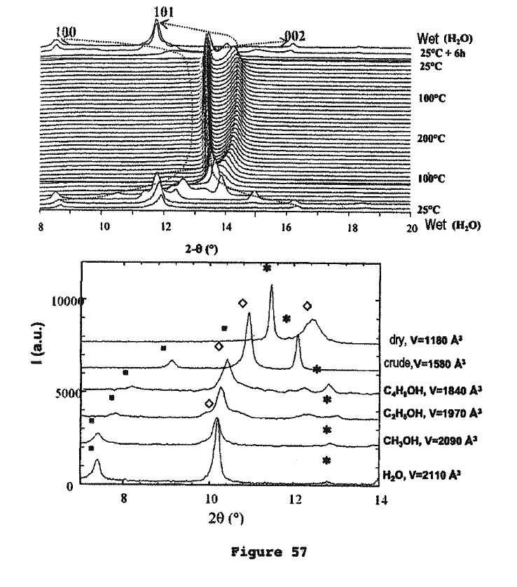

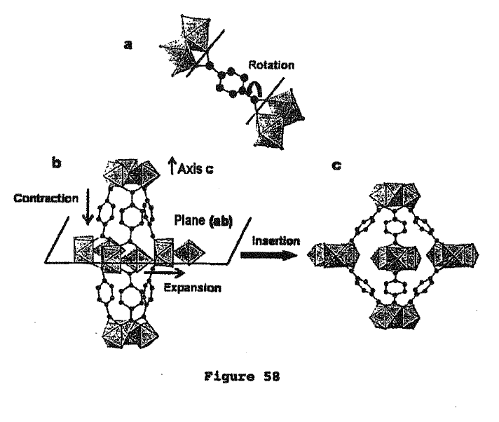

A study of the behavior of these solids by X-ray diffraction made it possible to establish that these compounds are flexible, with considerable “respiration” (i.e. swelling or contraction) amplitudes between their dry form and their solvated form. This results in variations in unit cell volume of between 85% and 230% depending on the nature of the organic spacer (FIG. 54), as described in the document by Serre et al., Science, 2007, 315, 1828 [18]. The inventors have noted that the dry forms are not porous with a more or less identical pore (tunnel) size irrespective of the carboxylic ligand used. On the other hand, the swelling of the hybrid solid in the liquid phase depends on the length of the organic spacer. Thus, the distance between trimers in the swollen form goes from 13.8 Å with fumaric acid (MIL-88A) to 20.5 Å with the biphenyl ligand (MIL-88D). The pore size of the swollen forms thus ranges between 7 Å (MIL-88A) and 16 Å (MIL-88D). The swelling is reversible, as shown by the example of the solid MIL-88A in the presence of water in FIG. 57, and also depends on the nature of the solvent used, as described in the document Serre et al. J. Am. Chem. Soc., 2005, 127, 16273-16278 [19]. “Respiration” takes place continuously, without apparent breakage of bonds during the respiration. Moreover, on returning to ambient temperature, the solid swells again by resolvation, confirming the reversible nature of the respiration.

If one takes a close look at the arrangement between the constituent trimers of the structure, each trimer is linked to six other trimers, three below and three above, via the dicarboxylates, which leads to the formation of bipyramidal cages of trimers. Within these cages, the connection between trimers is made solely along the axis c and the absence of any bond in the plan (ab) is the origin of the flexibility (FIG. 58).

TABLE 12

| | | | | | Unit | Estimated | |
|---|---|---|---|---|---|---|---|
| Solid | Condition | a (Å) | c (Å) | v (Å³) | cell expansion | pore size | Solvent |
| MIL-88A | 100° C. | 9.6 | 14.8 | 1180 | >80% | approximately | Water |
| | 25° C. | 11.1 | 14.5 | 1480 | | 6 Å | |
| | Open form | 13.8 | 12.5 | 2100 | | | |
| MIL-88B | 100° C. | 9.6 | 19.1 | 1500 | >100% | approximately | Ethanol |
| | 25° C. | 11.0 | 19.0 | 2000 | | 9 Å | |
| | Open form | 15.7 | 14.0 | 3100 | | | |
| MIL-88C | 100° C. | 9.9 | 23.8 | 2020 | >170% | approximately | Pyridine |
| | 25° C. | 10.2 | 23.6 | 2100 | | 13 Å | |
| | Open form | 18.7 | 18.8 | 5600 | | | |
| MIL-88D | 100° C. | 10.1 | 27.8 | 2480 | >220% | approximately | Ethanol |
| | 25° C. | 12.1 | 27.5 | 3500 | | 16 Å | |
| | Open form | 20.5 | 22.4 | 8100 | | | |

Specifically, when a solvent is inserted into the material, the cage becomes deformed, with approach of the trimers along the axis c and distancing in the directions a and b, which causes an overall increase in the volume of the cage (FIG. 58). Finally, the flexibility of these hybrid solids is noteworthy, but, however, comparable to that of certain polymers. The main difference concerns the crystallinity of the hybrid solids, polymers being amorphous. Finally, in contrast with polymers, the swelling takes place anisotropically in the hybrid solids.

TABLE 13
“MIL” structures of some iron (III)
carboxylates according to the invention

| Nanosolid | | |
|---|---|---|
| MIL-n | Organic fraction | Formula |
| MIL-88A | Fumaric acid | Fe₃OX[O₂C—C₂H₂—CO₂]₃—nH₂O |
| MIL-88B | Terephthalic acid | Fe₃OX[O₂C—C₆H₄—CO₂]₃•nH₂O |
| MIL-89 | Muconic acid | Fe₃OCl[O₂C—C₄H₄—CO₂]₃—nH₂O |
| MIL-100 | 1,3,5-benzene- | Fe₃OX[C₆H₃—[CO₂]₃]•nH₂O |
| | tricarboxylic | |
| | acid | |
| | (1,4-BTC acid) | |
| MIL-101 | Terephthalic acid | Fe₃OX[O₂C—C₆H₄—CO₂]₃•nH₂O |

TABLE 14
Characteristics of the iron (III) carboxylate
“MIL” structures

| | | Pore | | |
|---|---|---|---|---|
| | | diameter | | |
| MIL-n | % iron* | (Å)** | Flexibility | Metal base |
| MIL-88A | 30.8% | 6 | yes | Octahedral |
| | | | | trimer |
| MIL-88B | 24.2% | 9 | yes | Octahedral |
| | | | | trimer |
| MIL-89 | 26.2% | 11 | yes | Octahedral |
| | | | | trimer |
| MIL-100 | 27.3% | 25-29 | no | Octahedral |
| | | | | trimer |
| MIL-101 | 24.2% | 29-34 | no | Octahedral |
| | | | | trimer |

*Theoretical % iron in the dry phase
**Pore size calculated on the basis of the crystallographic structures

### Example 13

### NO Adsorption and Release by Iron-Based MOFs

Tests for encapsulation and release were carried out with some MOFs having various characteristics: rigid with high capacity (MIL100-Fe), having a redox activity (MIL100-Fe), flexible (MIL88), flexible and substituted (on the ligand) (MIL88-FeNO₂).

Specifically, while the delivery of NO in large amounts over a short period of time is easy to produce with zeolites, the porous hybrid solids of MIL-n type based on metals with a high oxidation state (+3) appear to have the ideal profile for sustained release of NO.

Specifically, the inventors have previously shown that the large-pore MOF denoted MIL-100, made up of octahedral trimers of chromium or on iron connected via trimesic acids, is stable, even after departure of the water coordinated on the metal centers. The latter is readily evacuated by heating under vacuum and gives way to unsaturated and accessible metal centers (metal in coordinance five). Most of the iron-based MIL solids have trimers of this type and can therefore all potentially adsorb, on their metal centers, organic molecules having an electron-donating nature (Lewis base), such as NO, via the free doublet located in the 5u orbitals.

### Nitric Oxide (NO) Loading:

The pre-activated MOF materials are exposed to approximately 2 bar of NO (99.5%, sold by the company Air Liquide) for 30 minutes. They are then evacuated under vacuum (in order to avoid the release of physisorbed NO and therefore the “initial burst” phenomenon corresponding to a very large amount of NO released in the first few minutes of release) and placed under a dry argon atmosphere. The latter operation (evacuation/argon) is repeated 3 times in order to be sure that all the physisorbed NO has been eliminated.

### Measurement of NO Adsorption/Desorption Isotherms

The NO adsorption/desorption measurements are carried out using an instrument of thermostated gravimetric type in order to eliminate any effect of the external environment. A CI microbalance, sold by the company CI Electronics Ltd, is used (sensitivity: 0.01 μg, reproducibility of the measurement of the mass: 0.1%). The pressure is measured by means of two BOC Edwards Active probes (measurement range: 1×10⁻⁸−1×10⁻² and 1×10⁻⁴−1×10⁻³ mbar). The MOF sample (−50 mg) is pre-activated at the required temperature (see above) at 2×10⁻³ mbar until no more loss of mass is observed. The sample is then cooled to the measuring temperature and stored at this temperature either by means of a thermostated waterbath (temperature accurate to ±0.02 K), or by dipping in liquid nitrogen. The counterbalance is maintained at the same temperature as the sample in order to minimize the effects of temperature difference on the temperature reading, itself measured using a type K thermocouple placed close to the sample (<5 mm). The variation in temperature of the sample during the measurement is less than 0.1 K. The dry NO gas (Air Liquide, 99.5%) is introduced into the system until the desired pressure is reached, and the increase in mass is measured as a function of time until stabilization occurs.

In this manner, an adsorption isotherm is obtained by incrementing the pressure and recording the gain of mass of the sample at equilibrium. The desorption of NO is carried out by gradually reducing the pressure to the desired value (2×10⁻³ mbar).

### Quantification of NO Release by Chemiluminescence

The NO measurements are carried out using a Sievers NOA 280i chemiluminescence Nitric Oxide Analyzer. The instrument is calibrated by passing air through a zero filter (Sievers, <1 ppb NO) and 89.48 ppm of NO gas (Air Products, balance nitrogen). The gas stream is fixed at 180 ml/min with a pressure in the cell of 8.5 torr and an oxygen pressure of 6.1 psi.

To measure the NO release by a sample in powder form, nitrogen gas with a known moisture content (11% water by passing the gas stream over an aqueous solution of LiCl) is passed over the powder and the resulting gas stream is sent to the analyzer, and the amount of NO (in ppm and ppb) is recorded. This approach is valid, for example, for cutaneous applications, where the solid is in contact with the skin and the NO release takes place in the presence of the moisture in the skin.

For applications where the solid is in contact with blood (tubes, catheters, etc.), the presence of an aqueous medium is necessary. For such applications, the NO release by a sample in powder form was studied in a simulated physiological fluid. Thus, the solid loaded with NO (50 mg+NO) is suspended in 4 ml of a saline phosphate buffer (pH ˜5.5; PBS) at 22° C. with stirring. The amount of NO released at various times is analyzed using the Sievers NOA 280i chemiluminescence Nitric Oxide Analyzer.

### 13.1. Solid MIL100(Fe)

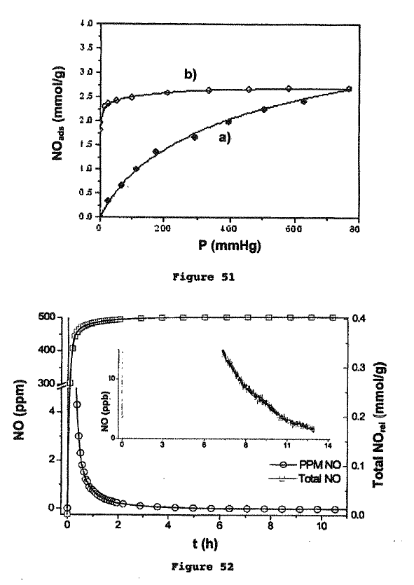

The solid MIL100(Fe) therefore adsorbs large amounts of NO (2-4 mmol·g⁻¹) at ambient temperature (approximately 20° C.) (FIG. 51) and releases it slowly and very partially (under an 11% moisture stream) as shown by the preliminary results obtained with the solids MIL-100(Fe) (FIG. 52). The profiles for release of NO under a water vapor pressure are, in addition, very advantageous.

FIG. 51 represents the NO adsorption isotherm (NO_{ads} in mmol/g) at the temperature of 298 K for the iron carboxylate MIL-100(Fe) activated at 120° C. overnight. This figure represents the amount of NO adsorbed (curve (a)) and desorbed (curve (b)) as a function of the pressure P (in mmHg).

FIG. 52 represents the profile for release of NO (NOᵣₑₗ in mmol/g) under a vapor pressure from the solid MIL-100(Fe). The NO is represented in ppm (or parts per million) or in ppb (or parts per billion) as a function of the time t in hours.

It is considered that NO release at a biologically useful level has finished when the rate of release is less than a few ppb/minute. It should also be noted that there is only a very partial release of all of the NO gas under these conditions (11% of water in a neutral gas stream) and that more or less 75% of the NO is still adsorbed. It will therefore remain to be seen at what speed this NO will become desorbed in real tests, i.e. when the solid is brought into contact with the physiological medium (on the skin or in contact with blood).

In a second step, the inventors partially reduced the iron(III) of the compound MIL-100(Fe) by activation under a primary vacuum (12 hours at 250° C.). The infrared spectroscopy showed that, under these conditions, this resulted in the reduction of approximately 15-20% of the iron(III) to iron(II).

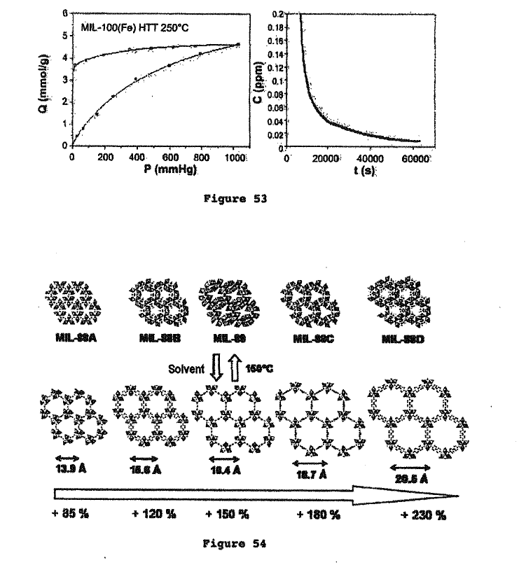

Transition metals of low oxidation states, such as iron(II), have additional electrons in the d orbitals. It is known that electron transfer from the d orbitals of the metal to the 2π* orbitals of molecules such as NO and CO reinforces the metal-adsorbate bond (phenomenon of back-donation) and thus stabilizes the species that are coordinated on the metal center. The amount of NO increases considerably with the introduction of iron(II) (4.5 mmol·g⁻¹ at 1 bar instead of 2.5 mmol·g⁻¹ for the pure iron(III) solid), since said iron(II) is capable of interacting with more than one molecule of NO per metal center (FIG. 53). Furthermore, the release is then slower, the release of NO still being present after 17 hours instead of 12 hours with the nonreduced analog. It should be noted here that the total amount of NO released (<1 mmol·g⁻¹) is once again much smaller than that adsorbed (2.5-4.5 mmol·g⁻¹). This comes from the presence of very strong sites of adsorption, which the water in vapor form (11% moisture content in the neutral gas) does not manage to desorb. Tests in an aqueous medium will make it possible to determine the real performance levels of these solids.

FIG. 53 represents, on the left: the NO adsorption isotherm at 298 K for MIL-100(Fe) activated at 250° C. under vacuum overnight; on the right: the profile for release of NO under a vapor pressure from the solid MIL-100(Fe) activated at 250° C. under vacuum overnight.

### 13.2 Solids MIL88A and MIL88B

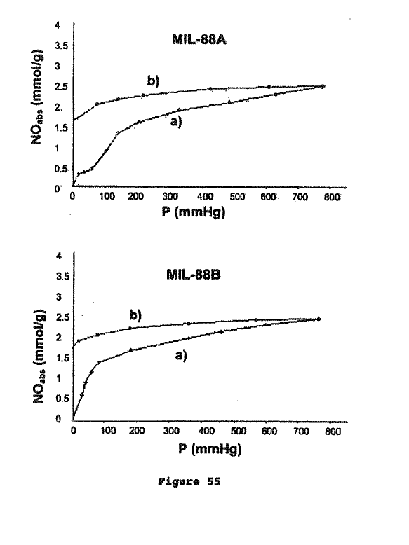

Finally, the inventors tested the adsorption and release of NO from the flexible phases MIL-88A and MIL-88B. These solids have the same octahedral trimers as the compound MIL-100, but with a flexible structure. It may be noted here that the inventors previously proved that these two compounds swell in the presence of liquids, but are not porous in their dry form (FIG. 54). It was therefore not at all obvious to think that these solids were going to adsorb NO gas. The inventors observed an adsorption of close to 2.5 mmol·g⁻¹ of NO at 298 K and at pressures below 1 atmosphere (FIG. 55).

FIG. 54 represents the schematic view of the respiration phenomenon in the solids MIL-88 (-A, -B, -C and -D).

FIG. 55 represents the NO adsorption isotherms at 298 K for the iron carboxylates MIL-88A(Fe) and MIL-88B(Fe) activated at 150° C. under vacuum overnight. The amount of NO (NO_{abs} in mmol/g) adsorbed (curve (a)) and desorbed (curve (b)) is represented as a function of the pressure P (in mmHg).

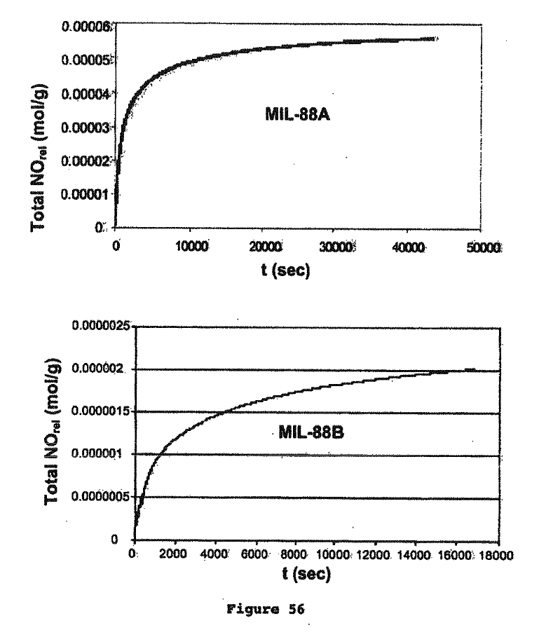

The release of NO is then tested under a water vapor pressure (11% moisture content in a neutral gas). In both cases, the amount of NO released is even lower than with the solid MIL-100 (<0.055 and 0.002 mmol·g⁻¹) in comparison with the amounts adsorbed (2.5 mmol·g⁻¹) (FIG. 56), which would imply, at first glance, that the gas is much more strongly adsorbed than on the compound MIL-100(Fe). This is the first time that such a low proportion of released NO is observed with a porous solid or a polymer.

FIG. 56 represents the profiles for release of NO under a water vapor pressure from the solids MIL-88A(Fe) (top) and MIL-88B(Fe) (bottom), activated at 150° C. overnight. The amount of NO released (NOᵣₑₗ in mol/g) is expressed as a function of the time t (in seconds).

For applications where the solid is in contact with blood (tubes, catheters, etc.), and therefore in the presence of an aqueous medium, this will no doubt result in an extremely slow release (a few days), which, in combination with the composition, which is a priori biocompatible, of these solids (iron, carboxylic acids), makes these solids highly advantageous for the desired applications. A possible explanation for this remarkable behavior could be the following: the flexible phases MIL-88 are “closed” after emptying of the pores by heating and therefore do not have accessible porosity for the usual gases (H₂, CO₂, CH₄, N₂, etc.). The N₂ adsorption measurements at 77 K previously showed the virtual absence of adsorption in these solids. The reason why NO gas is nevertheless adsorbed in a large amount, no doubt on the unsaturated metal centers, is because the interaction between this gas and the iron is much stronger than with the other types of gas molecules, in any event sufficient to slightly “open” the material. At this stage, NO entered the solid chemisorbed on the metal center, but the latter, having only very slightly open their pores, will make it very difficult for the water to diffuse in the pores and it is therefore extremely difficult for this water to drive the NO out of the solid. In an aqueous medium, during real tests, the release of NO will no doubt be conditioned by the hydrophobicity and the stability of these flexible phases. The possibility of changing almost at will the organic spacer of the phases MIL-88 will therefore in theory enable us to modulate the NO release kinetics in a physiological medium.

The release was also studied under conditions closer to real conditions in the plasma. Thus, the NO-loaded solid was placed in 4 ml of a phosphate buffer (pH ˜5.5) at 22° C. with stirring.

FIG. 71 represents the profiles for release of NO under a water vapor pressure (curve (a)) and in the phosphate buffer (curve (b)), from solid MIL-88A(Fe). The amount of NO released (NOᵣₑₗ in mmol·g⁻¹, on the left, and ppm NO, on the right) is expressed as a function of the time t (in hours).

The amount released is much greater when the release occurs in PBS than when it occurs under a water vapor stream, which is reasonable considering that the contact with the liquid medium is greater (FIG. 71). In this manner, both the water and the phosphates present in the medium will be able to displace the gas adsorbed/coordinated to metal.

There is a strong release of NO in the first 2 hours and, subsequently, the release is maintained at biologically active concentrations (>10 ppb) for up to 20 hours. This may be advantageous for having a shock effect initially (anticoagulant, for example) and maintaining it over a few hours.

With regard to the solid MIL-88B(Fe), the amount released is lower than that released for MIL-88A(Fe). The amount of NO released is very low (0.002 mmol·g⁻¹), whether in the gas stream or in the PBS (FIG. 58). Release at active concentrations is very rapid (1 hour in PBS and 4 hours in the gas stream).

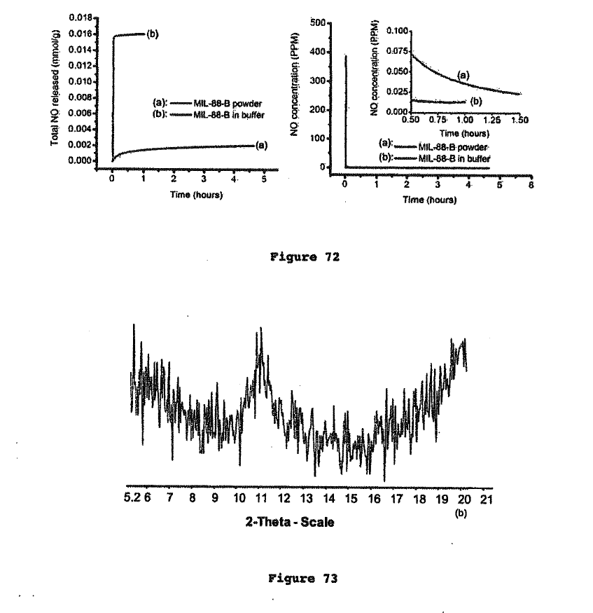

FIG. 72 represents the profiles for release of NO under a water vapor pressure (curve (a)) and in the phosphate buffer (curve (b)), from solid MIL-88B(Fe).

The amount of NO released (NOᵣₑₗ in mmol·g⁻¹, on the left, and ppm NO, on the right) is expressed as a function of the time t (in hours).

13.3. Solid MIL-88A-nano Fe₃O[(C₄H₂—(CO₂)₂]₃.X.nH₂O (X═F, Cl, OH)

### Synthesis

The microwave synthesis conditions are as follows:

270 mg (1 mmol) of FeCl₃.6H₂O, 116 mg of fumaric acid (1.0 mmol, Acros, 99%) dispersed in 30 ml of distilled water, the whole left in a Teflon body for 2 min at 100° C. with a heating ramp of 1 min (power 1600 W).

The solid is recovered by centrifugation at 10 000 rpm for 10 min.

200 mg of the product are suspended in 100 ml of distilled water in order to exchange the fumaric acid which remains free. The hydrated solid is recovered by centrifugation at 10 000 rpm for 10 min.

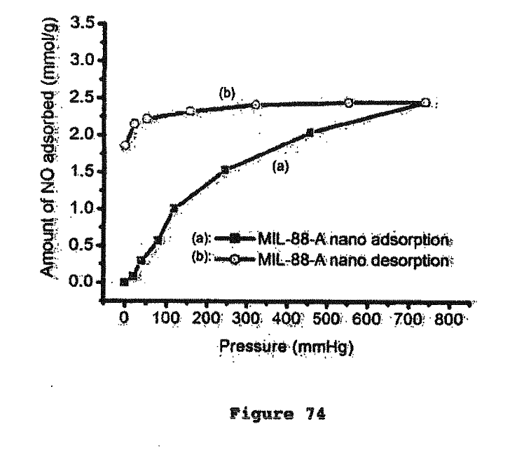

FIG. 73 represents the X-ray diffractogram of the solid MIL-88A-nano obtained by microwave synthesis.

The monodisperse particle size measured by light scattering is 120 nm.

### NO Adsorption

50 mg of MIL-88A-nano nanoparticles pre-activated under vacuum at ambient temperature (approximately 20° C.) for 5 h and under vacuum at 150° C. for 15 hours are exposed to approximately 2 bar of NO (99.5%, sold by the company Air Liquide) for 30 minutes (see example 13).

The amount of NO adsorbed, 2.5 mmol·g⁻¹, is considerable and entirely compatible with that obtained for the same structure with a larger crystallite size (previous example, particle size ˜5 microns).

FIG. 74 represents the NO adsorption isotherms at 298 K for the iron carboxylates MIL-88A(Fe)-nano activated at 150° C. under vacuum overnight. The amount of NO (NO_{abs} in mmol·g⁻¹) adsorbed (curve (a)) and desorbed (curve (b)) is represented as a function of the pressure P (in mmHg).

### No Release

The NO release is then tested under a water vapor pressure (11% moisture content in a neutral gas).

FIG. 75 represents the profiles for release of NO under water vapor pressure from the solids MIL-88A(Fe)-nano (120 nm, curve (b)) and MIL-88A(Fe) (5 microns, curve (a)). The amount of NO released (NOᵣₑₗ in mmol·g⁻¹, on the left, and ppm NO, on the right) is expressed as a function of the time t (in hours).

In the two cases, nano and micrometric materials, the amount of NO released is comparable (0.055 mmol·g⁻¹, FIG. 75). Micrometric MIL-88A(Fe) appears to have a slower release than MIL-88A(Fe)-nano in the first 10 hours. This effect is probably due to a smaller characteristic NO diffusion distance in the MIL88A nanoparticles compared with the micrometric MIL88A.

13.4 Solids MIL88B Modified with Various Functional Groups

The MIL-88B flexible crystal structure exhibits isotypes through the modification of the organic spacer with various functional groups. Thus, these functional groups will replace one or more hydrogens of the organic spacer (terephthalic acid), thus modulating the hydrophobicity and the stability of these flexible phases, and consequently the adsorption and release of biological gases. Furthermore, the electron-accepting groups will perhaps be able to create new interactions with the biological gases (Lewis bases).

The inventors tested the NO adsorption of and the NO release from the flexible phases of the MIL-88B type, based on the organic ligands: nitroterephthalate (MIL88B-NO₂) and 2,5-dihydroxyterephthalate (MIL88B-2OH).

### NO Adsorption

The two materials showed a similar adsorption capacity (1 mmol·g⁻¹), which was reduced compared with the unmodified solid MIL-88B. This effect can be explained by the incomplete removal of the coordinated water.

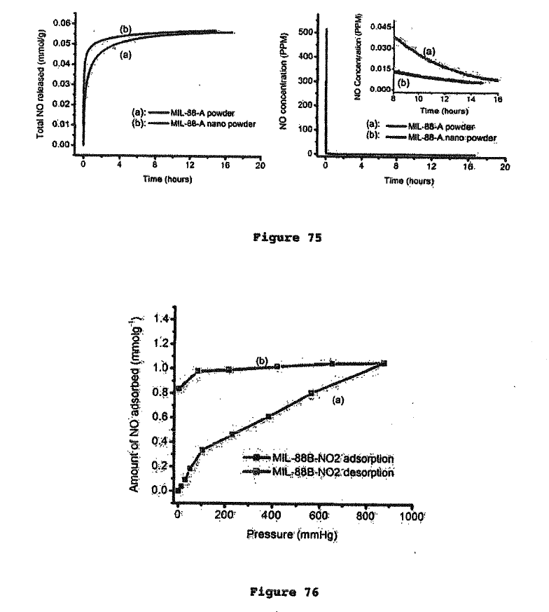

FIG. 76 represents the NO adsorption isotherms at 298 K for the iron carboxylates MIL-88B(Fe)-NO₂ activated at 150° C. under vacuum overnight. The amount of NO (NO_{abs} in mmol·g⁻¹) adsorbed (curve (a)) and desorbed (curve (b)) is represented as a function of the pressure P (in mmHg).

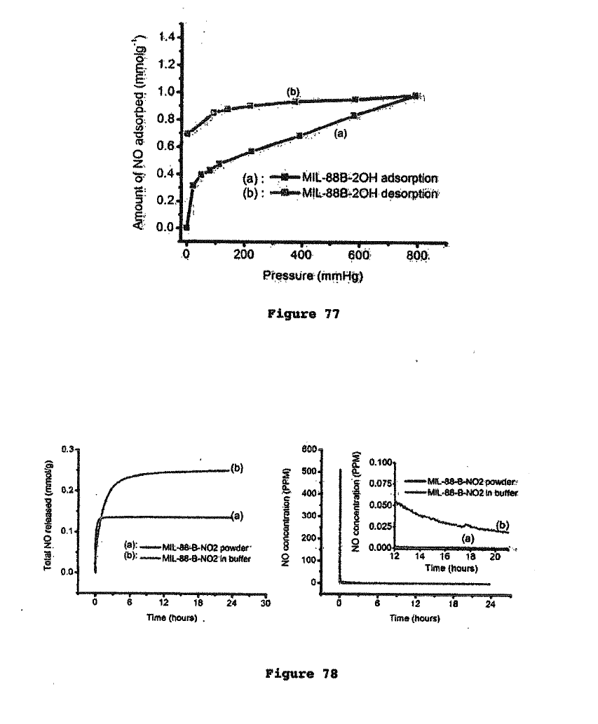

FIG. 77 represents the NO adsorption isotherms at 298 K for the iron carboxylates MIL-88B(Fe)-2OH activated at 80° C. under vacuum overnight. The amount of NO (NO_{abs} in mmol·g⁻¹) adsorbed (curve (a)) and desorbed (curve (b)) is represented as a function of the pressure P (in mmHg).

### NO Release

The functionalization with nitro or dihydroxy groups in the solid of the MIL-88B type makes it possible to considerably slow down the release of NO in the PBS medium.

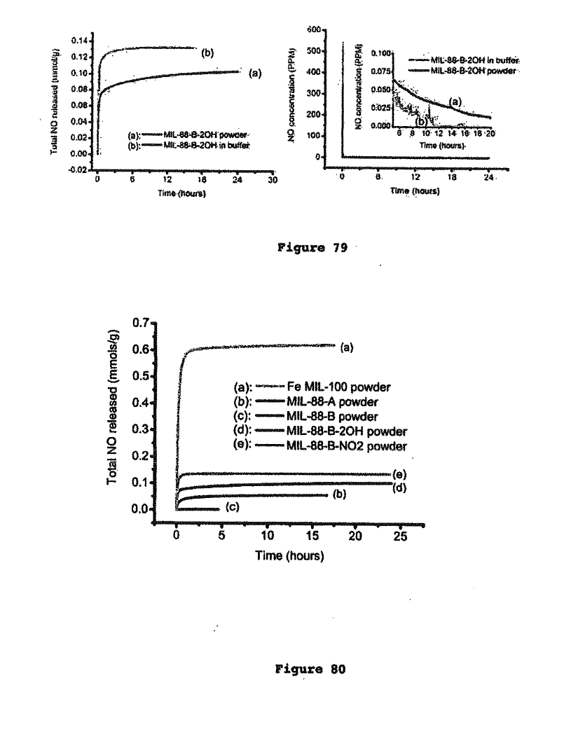

Thus, NO release is observed at biologically active concentrations (>10 ppm) for up to 11 and 24 hours with MIL-88B-2OH and MIL-88B-NO₂, respectively (FIGS. 78 and 79), in comparison with the unmodified solid MIL-88B, which releases its load in 1 hour. Similarly, the amount of NO released is also greater in MIL-88B-2OH and MIL-88B-NO₂ (0.13 and 0.25 mmol·g⁻¹, respectively) compared with the unmodified MIL-88B (0.01 mmol·g⁻¹).

It is observed that, when the NO release is successfully concluded in the PBS solution, the amount released is much greater than under gas stream conditions (table 15).

FIG. 78 represents the profiles for release of NO under a water vapor pressure (curve (a)) and in the phosphate buffer (curve (b)) from solid MIL-88B(Fe)-NO₂. The amount of NO released (NOᵣₑₗ in mmol·g⁻¹, on the left, and in ppm NO, on the right) is expressed as a function of the time t (in hours).

FIG. 79 represents the profiles for release of NO under a water vapor pressure (curve (a)) and in the phosphate buffer (curve (b)), from solid MIL-88B(Fe)-2OH. The amount of NO released (NOᵣₑₗ in mmol·g⁻¹, on the left, and in ppm NO, on the right) is expressed as a function of the time t (in hours).

### Comparison of the MILs

In view of the results (table 15 and FIG. 80), it can be concluded that it is possible to modulate the adsorption and release capacity and kinetics of the iron carboxylates as a function of their rigid, flexible or functionalized nature. While the mesoporous solid adsorbs and releases the greatest amounts of NO, the flexible phases adsorb less but release with very different kinetics as a function of the ligand chosen. Thus, the unmodified solids very rapidly (<1 h) release a very small amount, while the introduction of a functionality makes it possible not only to increase the amount released, but to do so over much longer characteristic times (11 and 24 h). This no doubt comes from the fact that it is much easier for the water to diffuse in these phases because of greater opening of the pores and from the fact that the substituents (OH, NO₂) are hydrophilic in nature.

TABLE 15
Adsorption and release capacity and kinetics
of the iron carboxylates in PBS solution and under gas
stream conditions

| | | NO | | | |
|---|---|---|---|---|---|
| | | released | Release | NO | Release |
| | | gas | time (h) | released | time (h) |
| | NO ads | stream | gas stream | PBS | PBS |
| | (mmol/g) | (mmol/g) | (>10 ppb) | (mmol/g) | (>10 ppb) |
| MIL88A | 2.5 | 0.055 | 18 | 0.5 | 20 |
| MIL-88A- | 2.5 | 0.06 | 15 | 0.01 | 1 |
| nano | | | | | |
| MIL88B | 2.5 | 0.002 | 4 | 0.016 | 1 |
| MIL88B- | 1.05 | 0.14 | 7 | 0.25 | 24 |
| NO₂ | | | | | |
| MIL88B- | 1 | 0.10 | 20 | 0.14 | 11 |
| 2OH | | | | | |
| MIL100 Fe | 2.5 | 0.4 | 10 | 0.4 | 24 |
| MIL100 | 4.5 | 0.6 | 15 | N/A | N/A |
| −250° C. | | | | | |
| MIL22 | 0.18 | 0.0016 | 1 | N/A | N/A |

FIG. 80 represents the profiles for release of NO under a water vapor pressure from the solids MIL-100Fe (curve (a)), MIL-88A (curve (b)), MIL-88B (curve (c)), MIL-88-2OH (curve (d)) and MIL-88B-NO₂ (curve (e)). The amount of NO released (NOᵣₑₗ in mmol·g⁻¹) is expressed as a function of the time t (in hours).

### Example 14

### NO Adsorption and Release Using the Titanium Diphosphonate MIL-22 (Ti₃O₂(H₂O)₂(O₃P—(CH₂)—PO₃)₂.(H₂O)₂)

The titanium diphosphonate MIL-22 was obtained according to the method reported by C. Serre, G. Férey, Inorg. Chem. 1999, 38, 5370-5373 [60].

50 mg of MIL-22, pre-activated under vacuum at 300° C. for 15 hours, are exposed to approximately 2 bar of NO (99.5%, sold by the company Air Liquide) for 30 minutes (see example 13).

The theoretical amount of NO adsorbed is ˜4 mmol·g⁻¹. On the other hand, the experimental NO adsorption capacity is only 0.18 mmol·g⁻¹. This difference can be explained since the activation conditions are insufficient to remove all the coordinated water. Thus, higher capacities may be obtained with conditions for activation of the solids such as 500° C. under vacuum for 16 hours.

FIG. 81 represents the NO adsorption isotherms at 298 K for the solid MIL-22 activated at 350° C. under vacuum overnight. The amount of NO(NO_{abs} in mmol·g⁻¹) adsorbed (curve (a)) and desorbed (curve (b)) is represented as a function of the pressure P (in mmHg).

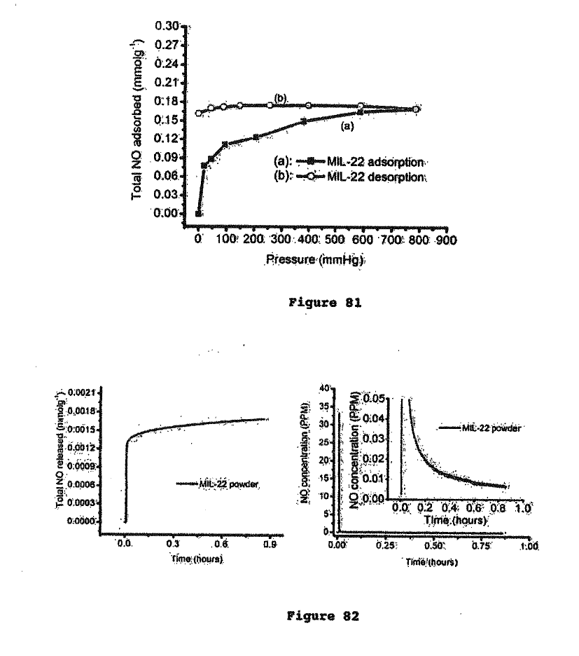

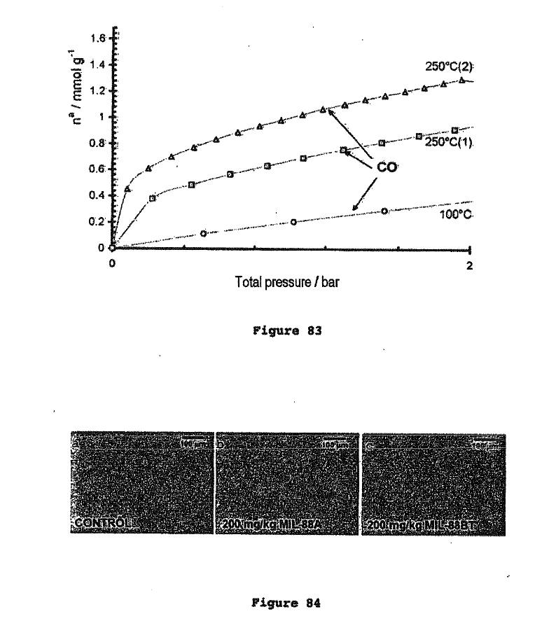

FIG. 82 represents the profiles for release of NO by the solid MIL-22 under a water vapor pressure. The amount of NO released (NOᵣₑₗ in mmol·g⁻¹ on the left and in ppm NO on the right) is expressed as a function of the time t (in hours).

Partial release of the NO (0.0016 mmol/g) takes place at biologically active concentrations for 0.8 hours.

### Example 15

### Measurement of the CO Adsorption Isotherms for the Solid MIL-100(Fe)

The CO adsorption measurements are carried out at 303 K up to 2 bar in a system for quantitatively determining gases, developed in the laboratory and connected to a thermostated gravimetric instrument (Rubotherm Prazisionsmeβtechnik GmbH). The MIL-100(Fe) sample (500 mg) is pre-activated at the required temperature (100° C. or 250° C.) under vacuum (at 2×10⁻³ mbar) for 12 or 20 hours. The dry CO gas (Air Liquide, 99.9%) is introduced into the system until the desired pressure is reached, and the increase in mass is measured as a function of the time until stabilization occurs.

In this manner, an adsorption isotherm is obtained by incrementing the pressure and recording the gain in mass of the sample at equilibrium.

FIG. 83 represents the CO adsorption isotherm (CO_{ads} in mmol/g) at the temperature of 303 K as a function of the pressure P (in bar) for the iron carboxylate MIL-100(Fe) activated at 100° C. for 12 h (100° C. curve), 250° C. for 12 h (250° C. (1) curve) and 250° C. for 20 h (250° C. (2) curve).

The solid MIL-100(Fe) adsorbs considerable amounts of CO at ambient temperature (0.4-1.3 mmol·g⁻¹) and at low pressure (up to 2 bar) (FIG. 83). The capacity of adsorption of CO in MIL-100(Fe) increases drastically when the iron(III) of the compound MIL-100(Fe) is partially reduced by activation at 250° C. under a primary vacuum (12 and 20 hours at 250° C.). The infrared spectroscopy showed that, under these conditions, this resulted in the reduction of approximately 15-20% of the iron(III) to iron(II). Transition metals of low oxidation states, such as iron(II), have additional electrons in the d orbitals. It is known that electron transfer from the d orbitals of the metal to the 2π* orbitals of molecules such as NO and CO reinforces the metal-adsorbate bond (phenomenon of back-donation) and thus stabilizes the species that are coordinated on the metal center. The amount of CO increases considerably with the introduction of iron(II) (1.3 mmol·g⁻¹ at 2 bar instead of 0.4 mmol·g⁻¹ for the pure iron(III) solid), because this iron(II) is capable of interacting with more than one molecule of CO per metal center.

### Example 16

### In Vivo Trials of Toxicity of the Iron(III) Carboxylates

### a) Iron Carboxylates Tested

The following two iron carboxylate solids (synthesized according to the procedures of example 1) are respectively tested:

3. MIL-88A(Fe) of composition Fe₂O[O₂C—C₂H₂—CO₂]₃—OH.nH₂O
4. MIL-88Btnano(Fe) of composition Fe₃O[O₂C—C₆(CH₃)₄—CO₂]₃.OH.nH₂O

### b) Toxicity Tests

The study of acute toxicity in vivo is carried out on 4-week-old female Wistar rats (125 g) by intravenously injecting into the rats increasing doses (50, 100 and 200 mg/kg) of MIL-88A nanoparticles (of 210 nm) and MIL-88Bt nanoparticles (of 100 nm) suspended in 0.5 ml of a 5% glucose solution.

The nanoparticles are stable in this medium.

The stability time of these suspensions is reduced to a few minutes when the particle concentration is at the maximum (200 mg/kg, 25 mg/0.5 ml). For this reason, the samples are taken under gentle stirring of the nanoparticle suspensions. It was not possible to administer doses higher than 200 mg/kg since the maximum volume that can be injected into rats is 0.5 ml.

The results are promising given that no major sign of toxicity is observed after 7 days of trials. The serum values for albumin, cholesterol and transaminases (ASAT/ALAT) do not show any significant variation after 7 days of trials, and the weight of the organs relative to the body weight does not vary significantly (table 16).

TABLE 16
Serum parameters measured 7 days after the
intravenous introduction of the iron carboxylates
MIL-88A(Fe) and MIL-88Bt(Fe)

| | | | | | Organ weight/total | | |
|---|---|---|---|---|---|---|---|
| | | Albumin | CHOL | ASAT/ | weight | | |
| | Dosage (mg/kg) | (g/l) | (mmol/L) | ALAT | Liver | Kidney | Spleen |
| Control | — | 44.2 | 2.5 | 2.5 | 0.041 | 0.009 | 0.004 |
| MIL-88A | 50 | 37.6 | 3 | — | 0.044 | 0.012 | 0.004 |
| MIL-88A | 100 | 46.0 | 2.2 | 2.5 | 0.041 | 0.009 | 0.004 |
| MIL-88A | 200 | 40.2 | 2.9 | 2.6 | 0.048 | 0.008 | 0.004 |
| MIL-88Bt | 50 | 39.5 | 2.5 | — | 0.048 | 0.010 | 0.003 |
| MIL-88Bt | 100 | 42.1 | 2.6 | 2.4 | 0.046 | 0.008 | 0.003 |
| MIL-88Bt | 200 | 38.5 | 2.6 | 2.5 | 0.044 | 0.008 | 0.004 |

The histological sections of the liver are observed by Proust staining (iron in blue), and presented in FIG. 84. They show an accumulation of iron in the liver. Although it is necessary to perform a more in-depth study on the long-term effects of these solids in the body, these results are very promising and make it possible to envision biomedical applications for these materials.

Acute and subacute toxicity studies were carried out in greater depth.

The animals used for the experiment are 4-week-old female Wistar rats weighing 161.36±16.1 g.

All the trials were carried out in the animal house of the University Pharmaceutical School under temperature and humidity conditions, and after 3 days of adapting the animals to the animal house (3 days).

For the acute toxicity tests, a single intrajugular injection of the materials MIL-88A (150 and 500 nm), MIL-88Bt (50 and 140 nm) or 5% glucose (control group) is given to 4 groups (at 1 day, 1 week, 1 month and 3 months, respectively) of 8 rats chosen at random and anesthetized with isoflurane.

The change in weight and the behavior of the animals were monitored.

Blood samples were also taken, from the jugular vein under anesthesia with isoflurane, at various times: 1 and 3 days, 1 and 2 weeks, 1, 2 and 3 months. The serum was isolated in order to measure serum parameters such as IL-6 (interleukin 6), albumin, serum Fe, PAS, GGT, bilirubin, cholesterol and transaminases.

Moreover, each group of animals was sacrificed after 1 day, 1 week, 1 and 3 months, respectively. The animals were anesthetized with isoflurane and then the spleen, the kidneys, the liver and the heart were removed and stored for histological studies. Four livers were also used to perform a microsomal extraction in order to measure cytochrome P450 activation.

For the subacute toxicity tests, one intrajugular injection per day is given for 4 consecutive days to 26 rats distributed at random in various groups, in which the animals are sacrificed after 5 or 10 days.

The change in weight of the isolated animals and also their eating behavior (measurement of the amounts of water and feed consumed) were monitored. The urine and dejecta were also recovered.

Blood samples were also taken, from the jugular vein, on various groups of rats at 3 and 5 days, and 8 and 10 days. The blood undergoes the same treatment as for the acute toxicity trial and the serum obtained is intended for the same analyses.

On the days of sacrifice, at 5 and 10 days, the animals are anesthetized with isoflurane and then the spleen, the kidneys, the liver, the heart and the lungs are removed and treated in the same way as for the acute toxicity trial.

### c) Results

### Weight Change of the Animals:

The animals were weighed every day for the purpose of comparing the weight change of the various groups. A mean was determined for each day and in each of these groups.

For the subacute toxicity tests, the increase in weight observed with the glucose group is slightly reduced when the material is administered. This variation is more obvious when the administered dose is higher.

The acute toxicity studies show that the administration of the materials MIL-88A and MIL-88Bt does not produce any significant variation in weight over time.

### Change in Consumption of Water and Feed:

In subacute toxicity, the change is similar overall for the control group and the group which received an injection of 25 mg/kg. A more pronounced difference is observed in the group which received the highest dose, and is characterized by a smaller consumption of feed during the study. This observation is confirmed and completely agrees with the results obtained for the weight change.

### Comparison of the Weight of the Removed Organs:

Subacute toxicity results: no significant difference appears between the weight of the spleen, kidneys and heart of the various groups. The weight of the lungs appears to be slightly increased both at 5 days and at 10 days.

Acute toxicity: an increase in the weight of the spleen is observed up to one week after the administration, returning to normal at 1 and 3 months for MIL-88A and MIL-88Bt, respectively. The weight of the liver increases substantially when the materials are injected, which possibly reflects the accumulation of iron in the liver. It is observed that the situation returns to normal for MIL-88A after 3 months, but not for MIL-88Bt, where the weight remains high.

### Assaying of Cytochrome P450 in Microsomal Suspensions:

Cytochrome P450 is an enzyme associated with the inner face of the smooth endoplasmic reticulum, which is highly involved in the degradation of exogenous molecules. This enzyme has a very low substrate specificity and is capable of catalyzing the transformation of newly synthesized compounds such as medicaments. The majority of P450 cytochromes can be induced or repressed, at the transcriptional level, by various xenobiotics; this is often the cause of side effects of medicaments. Assaying this enzyme makes it possible to determine whether the MOF material used is metabolized by cytochrome P450, in which case the latter would activate or inhibit the activity of said material.

The amount of cytochrome can be interpreted only on condition that it has been related to the total amount of proteins contained in each sample. The assaying of proteins contained in the sample was carried out using a BCA kit supplied by Pierce (batch #HI106096). This method combines the reduction of Cu²⁺ to Cu⁺ by the proteins in an alkaline medium with very sensitive and selective colorimetric detection of the Cu⁺ cation by means of a reactant containing bicinchoninic acid (BCA).

The relationship between the concentration of cytochrome and the total amount of proteins gives the activity of the cytochrome, expressed in mol·g⁻¹. The acute toxicity results show that there is no major difference in activity between the negative control group (which received glucose) and the “MIL-88A” group, the material of which is not metabolized by Cyp450. The material MIL-88Bt also does not appear to be metabolized by Cyp450.

### Assaying of Interleukin 6 in the Serum:

Interleukin 6 (IL-6) is a multifunctional cytokine which plays an important role in the host's defense, immune responses, nerve cell functions and hematopoiesis. An elevated IL-6 level in the serum has, for example, been observed during viral and bacteriological infections, trauma, autoimmune diseases, inflammation or cancer.

The aim of this study is to determine whether there is an inflammatory reaction after administration of the iron carboxylate nanoparticles. Thus, it is possible to see whether the IL-6 level is increased compared with the control groups (injection of glucose, and therefore local inflammatory reaction due to the injection).

The assay was carried out by using a “Quantikine, Rat IL-6” kit supplied by R&D Systems laboratories.

Subacute toxicity results: the variations are not significant. An increase in the plasma level observed (activation of IL-6 production) appears to be due to an injection phenomenon which occurs with injections that produce a local inflammation, if the various groups are compared in isolation with the control group (glucose).

Acute toxicity results: the variations are not significant and lead to the same conclusions as in the case of the subacute toxicity.

### Assaying of Serum Parameters:

All the assays were carried out using automatic devices. Some key parameters were determined in order to evaluate the consequences of the nanoparticle injections at the level of the liver, the levels of transaminases (alanine aminotransferase or ALAT and aspartate aminotransferase or ASAT), alkaline phosphatases (PAS), γ-glutamate transferase (GGT), bilirubin, cholesterol, albumin and serum iron.

The results show that the serum levels of ALAT are entirely normal, as are the levels of bilirubin (<2 μmol/l) and γ-glutamate transferase (<2 IU/l).

The serum albumin levels were slightly reduced after the first day of injection for the two materials, in agreement with a local inflammatory process due to the injection, and with the increase in IL-6 observed previously. After 3 days, the levels return to normal.

The serum ASAT levels are increased one day after the injection, which may indicate a cytolytic process. However, 3 days after the administration of the nanoparticles, the values return to normal. Similarly, the alkaline phosphatase is increased after 1 day, indicating a cytolytic process, but the situation returns to normal after 3 days. The return to normal after 3 days indicates that it is a transient rather than permanent cytolytic process. There is therefore no loss of cell function.

The cholesterol levels are normal.

The serum iron levels are decreased in comparison with the control group, and this is more pronounced in the MIL-88A group. This might be explained by complexation of the serum iron by the nanoparticles. The situation returns to normal 3 days after the administration.

The serum parameters were also assayed at 1 week and, according to these results, there is no difference between the 3 groups as regards the serum iron; the rats treated with MIL-88A and MIL-88Bt recovered a serum iron concentration comparable to that of the control group. Moreover, as regards the levels of the other serum parameters, there is no significant difference in comparison with the control group.

### Histological Sections:

Histological sections 5 μm thick are cut in a cryostat, dehydrated and stained (hematoxylin/eosin staining then staining with Proust blue: blue staining of the iron).

By observing the histological sections, it is possible to determine the route of elimination of the compounds of the material or their storage in certain organs: liver, kidneys, spleen and lungs, the heart being used as a control.

Acute toxicity results: the liver histological sections show an accumulation of iron in the liver after injection of the materials, which is higher for the solid MIL-88A. The material appears to be in the form of nondegraded nanoparticles. The accumulation is smaller for the material MIL-88Bt, which may mean less uptake for the liver or more rapid re-use of the stored iron. After 1 and 3 months, the iron content in the spleen and the liver returns to normal.

### Assaying of Iron in the Injected Suspensions and in the Organs:

The assaying of the iron contained in the suspensions of MIL-88A and MIL-88Bt injected into the animals is carried out by UV-visible spectrophotometry at the wavelength of 520 nm, by specific colorimetry of the ferrous ions with bipyridine (formation of a red complex), after solubilization of the iron oxide in concentrated sulfuric acid, and reduction of the ferric ions to give ferrous ions with ascorbic acid.

The assaying of the iron in the organs is carried out in the same way as the assaying of iron in the suspensions, explained above, after grinding the organ to be tested. This assay makes it possible to determine the route of elimination of the compounds of the material or their storage in certain organs: liver, kidneys, spleen and lungs, the heart being used as a control.

### d) Conclusion

During the toxicity trials, minute observation of the animals revealed no apparent sign of harmfulness of the injected material. Specifically, the animals maintained entirely normal behavior. During the studies, the animals put on weight well, in comparison with the control group, even though, for the subacute toxicity study, the increase in weight is smaller than for the control group, probably associated with the consecutive administration of high doses. The water consumption itself remains normal on the whole in the subacute toxicity trial.

Assaying of cytochrome P450 made it possible to observe the state of activity of cytochrome P450 over a long period. This cytochrome is known for its ability to metabolize certain xenobiotics. The study shows that the activity level, although subject to fluctuation, remains below the values observed on the control rats which received an injection of phenobarbital, a cytochrome P450 activator, which indicates that the materials are not metabolized via the Cyp450 pathway, which is in agreement with the high polarity of the dicarboxylic ligands.

The results are very promising and already indicate that the materials MIL-88A and MIL-88Bt do not induce any sign of severe toxicity, although complementary toxicity studies should be carried out. The fate and the effects of the nanoparticles in the body are in the process of being studied in order to bring together the benefit provided by these materials through the vectorization of medicaments that are difficult to encapsulate and that are of great therapeutic potential. Similar studies are also underway with other nanovectors of different structure and/or composition.

### Example 17

### Comparison of Performance Levels of Various Porous Solids with Respect to NO Adsorption and Release

The MOF materials have many advantages compared with inorganic porous solids, zeolites. Thus, the high stability of zeolites in aqueous media does not allow elimination thereof by the organism, producing storage of the product in the body. Furthermore, most zeolites have aluminum in their structure, an element with a very high toxicity.

Similarly, the composition of other MOFs, based on metals well known for their toxicity (cobalt, nickel, chromium or copper) does not make it possible to envision applications in the medical or cosmetics field for these MOFs. Unlike these MOFs, the solids of the invention have a composition which is, a priori, not very toxic (Fe, Ti). Specifically, in example 16, we demonstrate the absence of toxicity of two iron carboxylates (iron fumarate and iron tetramethyl-terephthalate) by means of in vivo toxicity tests on rats.

Furthermore, with the solids of the invention, it is possible to control their rate of degradation according to the applications desired. For example, the solid MIL-100(Fe) is stable under hydrothermal conditions, while the solid MIL-101(Fe) is rapidly degraded in the presence of water at ambient temperature.

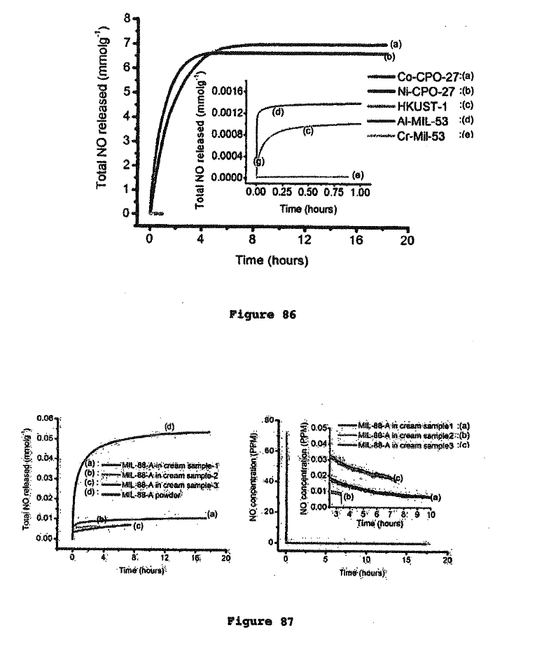

The performance levels of our MOFs were also compared with the MOFs reported in the literature (including in application WO 2008/020218). With regard to their adsorption capacity, the materials reported in this invention showed capacities entirely comparable to those of the other MOFs, based on toxic metals (4.5 mmol·g⁻¹) (FIG. 84), with releases controlled over time. Furthermore, we obtained the iron(II)-based solid CPO-27 (iron 2,5-dihydroxoterephthalate), of biocompatible composition, the expected NO adsorption capacity of which will be close to that of the Ni-based and/or Co-based analogs (6-7 mmol·g⁻¹) with a controlled release over a very long period of time (FIG. 86).

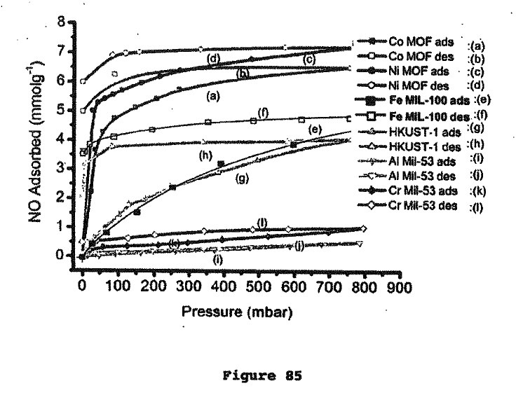

FIG. 85 represents the NO adsorption isotherms at 298 K for the solids CPO-27 (Co dihydroxoterephthalate) (curves (a) and (b)), CPO-27 (Ni 2,5-dihydroxoterephthalate; M₂(dhtp)(H₂O).xH₂O (M=Ni or Co, dhtp=2,5-dihydroxyterephthalic acid, x˜8)) (curves (c) and (d)), MIL-100 (Fe trimesate) (curves (e) and (f)), HKUST (Cu trimesate) (curves (g) and (h)), MIL-53 (Al terephthalate) (curves (i) and (j)) and MIL-53 (Cr terephthalate) (curves (k) and (l)).

The amount of NO adsorbed (NO_{abs} in mmol·g⁻¹) and desorbed is represented as a function of the pressure P (in mbar).

FIG. 86 represents the profiles for release of NO under a water vapor pressure from the solid CPO-27 (Co dihydroxoterephthalate) (curve (a)), CPO-27 (Ni 2,5-dihydroxoterephthalate; M₂ (dhtp) (H₂O).xH₂O (M=Ni or Co, dhtp=2,5-dihydroxyterephthalic acid, x˜8)), (curve (b)), HKUST-1 (Cu trimesate) (curve (c)), MIL-53 (Al terephthalate) (curve (d)) and MIL-53 (Cr terephthalate) (curve (e)). The amount of NO released (NOᵣₑₗ in mmol·g⁻¹) is expressed as a function of the time t (in hours).

### Example 18

### Formulation in the Form of a Cream Comprising an MOF Solid According to the Invention

The cream used is composed of 50% by weight of paraffin and 50% by weight of polyethylene glycol (PEG), the two being mixed using an automatic pipette for 30 seconds.

10% by weight of the solid MIL-88A or MIL-88A-nano, loaded with NO, are then mixed in the same manner with the cream.

In order to measure the release of NO by a sample in the form of a cream, nitrogen gas with a known moisture content (11% water by passing the gas stream over an aqueous solution of LiCl) is passed over the mixture of cream and of powder; the resulting gas stream is then sent to the analyzer, and the amount of NO (in ppm and ppb) is recorded. This approach is valid for cutaneous applications, where the solid is in contact with the skin and the release of NO occurs in the presence of the moisture in the skin.

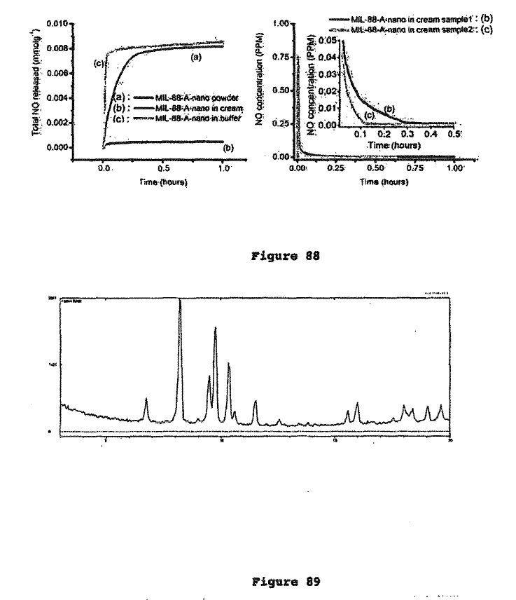

The amount of NO released by the cream is 6-8 times smaller than the powder form (FIGS. 87 and 88) because the paraffin, which is hydrophobic, does not allow contact between the solid and the water vapor stream.

Release at biologically active concentrations takes place for 10 hours in the case of the solid MIL-88A of micrometric size (FIG. 87). On the other hand, a very rapid release is observed (10 minutes; FIG. 88) when the cream comprises MIL-88A-nano. These results, which are entirely in agreement with those obtained with the powder, are due to a smaller diffusion length and also to better contact of the cream by virtue of the larger surface area of these nanoparticles.

FIG. 85 represents the profiles for release of NO under a water vapor pressure from the solid MIL-88A (3 samples under the same conditions) in the form of a cream. The amount of NO released (NOᵣₑₗ in mmol·g⁻¹) is expressed as a function of the time t (in hours).

FIG. 86 represents the profiles for release of NO under a water vapor pressure from the solid MIL-88A-nano in the form of a cream (curve (b)) and in the form of a powder (curve (a)) in comparison with the release in a PBS solution (curve (c)). The amount of NO released (NOᵣₑₗ in mmol·g⁻¹) is expressed as a function of the time t (in hours).

### LIST OF REFERENCES

[1] Patent application U.S. Ser. No. 10/039,733
[2] Patent application U.S. Ser. No. 10/061,147
[3] Patent application U.S. Ser. No. 10/137,043
[4] Nitric oxide function in the skin, Cals-Grierson M. M., Ormerod A D, NITRIC OXIDE-BIOLOGY AND CHEMISTRY 2004, 10(4), 179-193 Topically applied nitric oxide induces T-lymphocyte infiltration in human skin, but minimal inflammation, Mowbray M, Tan X. J. Wheatley P. S., et al., JOURNAL OF INVESTIGATIVE DERMATOLOGY 2008, 128(2), 352-360
[5] Effects of nitric oxide and oxidation in vivo and postmortem on meat tenderness, Warner R. D., Dunshea F. R., Ponnampalam E. N., et al. MEAT SCIENCE volume: 71 issue: 1 pages: 205-217, 2005
[6] L. K. Keefer Nature Materials, 2, 357 (2003)
[7] P. G. Parzuchowski et al., J. Am. Chem. Soc. 124, 12182 (2002)
[8] J. T. Mitchell-Koch et al., Angew Chemie, 43, 2806 (2004)
[9] A. D. Ormerod et al., J. Invest Dermatol 113, 392-397 (1999)
[10] WO 2005/003032
[11] P. S. Wheatley et al., Stud Surf Sci Catal, 158, 2033-2040 (2005); P. S. Wheatley et al., J. Am. Chem. Soc., 128, 502-509 (2006) B. Xiao et al., J. Am. Chem. Soc., 129, 1203-1209 (2007)
[12] Pollution fighter turns clot buster, New Scientist, 5^{th} Feb. (2005)
[13] M. Mowbray et al., J. Invest Dermatol, 126, 102 (2006)
[14] C. T. Dziobkowski, T. J. Wrobleski, D. B. Brown, Inorg. Chem. 1982, 20, 671
[15] L. Anzalone et al., J. Org. Chem. 1985, 50, 2128
[16] S. Ameerunisha et al., J. Chem. Soc. Perkin Trans. 2 1995, 1679
[17] C. Serre et al., Angew. Chem. Int. Ed. 2004, 43, 6286 C. Serre et al., Chem. Comm. 2006, 284-286 [29]
[18] Serre et al., Science, 2007, 315, 1828 [30]
[19] C. Serre et al. J. Am. Chem. Soc., 2005, 127, 16273-16278
[20] K. Byrapsa, M. Yoshimura, “Handbook of hydrothermal technology”, Noyes Publications, Parkridge, N. J., USA, William Andrew Publishing, LLC, Norwich N.Y. USA, 2001
[21] G. Tompsett, W. C. Conner, K. S. Yngvesson, Chem Phys Chem. 2006, 7, 296
[22] S.-E. Park, J.-S. Chang, Y. K. Hwang, D. S. Kim, S. H. Jhung, J.-S. Hwang, Catal. Survey Asia 2004, 8, 91
[23] C. S. Cundy, Collect. Czech. Chem. Commun. 1998, 63, 1699
[24] S. H. Jhung, J.-H. Lee, J.-S. Chang, Bull. Kor. Chem. Soc. 2005, 26, 880
[25] A. Pichon, Cryst. Eng. Comm. 8, 2006, 211-214
[26] D. Braga, Angew. Chem. Int. Ed. 45, 2006, 142-246
[27] D. Braga, Dalton Trans., 2006, 1249-1263
[28] R. Alberto and R. Motterlini, Dalton Trans, 2007, 1650
[29] Sabine Balthasar, Kerstin Michaelis, Norbert Dinauer, Hagen von Briesen, Jörg Kreuter and Klaus Langer, “Preparation and characterisation of antibody modified gelatin nanoparticles as drug carrier system for uptake in lymphocytes”, Biomaterials, 2005, 26, 15, 2723-2732
[30] Ruxandra Gref, Patrick Couvreur, Gillian Barratt and Evgueni Mysiakine, “Surface-engineered nanoparticles for multiple ligand coupling”, Biomaterials, 2003, 24, 24, 4529-4537
[31] Keefer L. K., Nat. Mater. 2003, vol. 2, 357
[32] A. R. Butler and R. Nicholson, Life, Death and Nitric Oxide, The Royal Society of Chemistry, Cambridge, 2003; F. Murad, N. Engl. J. Med., ‘Nitric Oxide and Cyclic GMP in Cell Signaling and Drug Development’ 2006, 355, 2003
[33] C. Napoli and L. J. Ignarro, ° NO-Releasing Drugs' Annu. Rev. Pharmacol. Toxicol., 2003, 43, 97
[34] T. R. Johnson, B. E. Mann, J. E. Clark, R. Foresti, C. Green and R. Motterlini, ‘Metal carbonyls: a new class of pharmaceuticals?’ Angew. Chem., Int. Ed., 2003, 42, 3722; S. W. Ryter, J. Alam and A. M. K. Choi, ‘CO-metal interaction: vital signalling from a lethal gas’ Physiol. Rev., 2006, 86, 583; J. Boczkowski, J. J. Poderoso and R. Motterlini, ‘CO-metal interaction: vital signalling from a lethal gas’ Trends Biochem. Sci., 2006, 31, 614: R. Motterlini, B. E. Mann and R. Foresti, ‘Therapeutic applications of carbon-monoxide-releasing molecules’ Expert Opin. Invest. Drugs, 2005, 14, 1305
[35] L. E. Otterbein, F. H. Bach, J. Alam, M. Soares, H. T. Lu, M. Wysk, R. J. Davis, R. A. Flavell, A. M. K. Choi, Nat. Med. 2006, 6, 422-428
[36] J. T. Chapman, L. E. Otterbein, J. A. Elias, A. M. K. Choi, Am. J. Physiol. Lung Cell Mol. Physiol. 2001, 281, L209-L215
[37] K. Sato, J. Balla, L. Otterbein, R. N. Smith, S. Brouard, Y. Lin, E. Csizmadia, J. Sevigny, S. C. Robson, G. Vercellotti, A. M. Choi, F. H. Bach, M. P. Soares, J. Immunol. 2001, 166, 4185-4194K
[38] M. Stupfel, G. Bouley, Ann. N.Y. Acad. Sci. 1970, 174, 342-368
[39] F. Amersi, X. D. Shen, D. Anselmo, J. Melinek, S. Iyer, D. J. Southard, M. Katori, H. D. Volk, R. W. Busuttil, R. Buelow, J. W. Kupiec-Weglinski, Heptaology 2002, 35, 815-823
[40] L. Gunther, P. O. Berberat, M. Haga, S. Brouard, R. N. Smith, M. P. Soares, F. H. Bach, E. Tobiasch, Diabetes 2002, 51, 994-999
[41] N. Ozawa, N. Goda, N. Makino, T. Yamaguchi, Y. Yoshimura, M. Suematsu, J. Clin. Invest. 2002, 109, 457-467
[42] S. A. Bainbridge, A. E. Farley, B. E. McLaughlin, C. H. Graham, G. S. Marks, K. Nakatsu, J. F. Brien, G. N. Smith, Placenta 2002, 23, 563-569
[43] D. Morse, J. Sethi, A. M. K. Choi, Crit. Care Med. 2002, 30, S12-S17
[44] S. P. Gaine, G. Booth, L. Otterbein, N. A. Flavahan, A. M. Choi, C. M. Weiner, J. Vasc. Res. 1999, 36, 114-119; T. Morita, S. A. Mitsialis, H. Koike, Y. Liu, S. Kourembanas, J. Biol. Chem. 1997, 272, 32 804-32809
[45] R. Motterlini, A. Gonzales, R. Foresti, J. E. Clark, C. J. Green, R. M. Winslow, Circ. Res. 1998, 83, 568-577; I. A. Sammut, R. Foresti, J. E. Clark, D. J. Exon, M. J. Vesely, P. Sarathchandra, C. J. Green, R. Motterlini, Br. J. Pharmacol. 1998, 125, 1437-1444
[46] L. E. Otterbein, B. S. Zuckerbraun, M. Haga, F. Liu, R. Song, A. Usheva, C. Stachulak, N. Bodyak, R. N. Smith, E. Csizmadia, S. Tyagi, Y. Akamatsu, R. J. Flavell, T. R. Billiar, E. Tzeng, F. H. Bach, A. M. K. Choi, M. G. Soares, Nat, Med. 2003, 9, 183-190
[47] D. E. Baranano, S. Dore, C. D. Ferris, S. H. Snyder, Clin. Neurosci. Res. 2001, 1, 46-52) et de l'ovulation (W. Tschugguel, F. Stonek, Z. Zhegu, W. Dietrich, C. Schneeberger, T. Stimpfl, T. Waldhoer, W. Vycudilik, J. C. Huber, J. Clin. Endocrinol. Metab. 2001, 86, 3833-3839
[48] M. McLean, M. Bowman, V. Clifton, R. Smith, A. B. Grossman, J. Clin. Endocrinol. Metab. 2000, 85, 2345-2349
[49] R. Wang, FASEB J., 2002, 16, 1792; R. Wang, Antioxid. Redox Signaling, 2003, 5, 493
[50] W. M. Zhoa, K. N. Houk and L. F. Olson, EMBO J., 2001, 20, 6008
[51] Y. Liu, J. F. Eubank, A. J. Cairns, J. Eckert, V. Ch. Kravtsov, R. Luebke, M. Eddaoudi, Angew. Chem. Int. Ed. 2007, 46, 3278-3283
[52] Robert A. Moss, Hugo Morales-Rojas, Saketh Vijayaraghavan and Jingzhi Tian, J. Am. Chem. Soc., 2004, 126, (35), 10923-10936
[53] B. Baleux et al. C. R. Acad. Sciences Paris, series C, 274 (1972) pages 1617-1620
[54] R. Gref et al. Colloids and Surfaces B: Biointerfaces, volume 18, issues 3-4, October 2000, pages 301-313
[55] Kohler N. et al., J Am Chem Soc 2004; 126; 7206-7211
[56] Peggy Chan et al., Biomaterials, volume 28, issue 3, 2007, pp 540-549
[57] J. H. Van Steenis et al., J Control Release 87 (2003), pp. 167-176
[58] A. Gabizon, H. Shmeeda, A. T. Horowitz and S. Zalipsky, Tumor cell targeting of liposome-entrapped drugs with phospholipids-anchored folic acid-PEG conjugates, Adv Drug Deliv Rev 56 (2004), pp. 1177-1192
[59] Ameerunisha et al., J. Chem. Soc. Perkin Trans. 2, 1679, 1995
[60] C. Serre, G. Férey, Inorg. Chem. 1999, 38, 5370-5373
[61] P. D. C. Dietzel, B. Panella, M. Hirscher, R. Blom, H. Fjellvag, Chem. Commun., 2006, 956-961 and P. D. C. Dietzel, R. E. Johnsen, R. Blom, H. Fjellvag, P. Chem. Eur. J., 2008, 14, 2389-2397

1. A porous crystalline MOF solid loaded with at least one Lewis base gas chosen from the group comprising, NO, CO and H₂S, at least a part of which coordinates with M, said solid comprising a three-dimensional succession of units having the following formula (I):
MₘOₖXₗLₚ (I)
in which:
each occurrence of M independently represents an ion of a transition metal M^{z+} chosen from the group comprising Fe, Ti, Zr and Mn and in which z is 2 to 4, or a mixture thereof;
m is 1 to 12;
k is 0 to 4;
l is 0 to 18;
p 1 to 6;
X is an anion chosen from the group comprising OH⁻, Cl⁻, F⁻, I⁻, Br⁻, SO₄²⁻, NO₃⁻, ClO₄⁻, PF₆⁻, BF₄⁻, R—(COO)ₙ⁻ where R is as defined below, R¹—(COO)ₙ⁻, R¹—(PO₃)ₙ⁻, where R¹ is a hydrogen, a linear or branched optionally substituted, C₁ to C₁₂ alkyl, or an aryl, and where n is an integer from 1 to 4;
L is a spacer ligand comprising a radical R composing q carboxylate groups 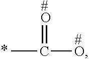
where
q is 1, 2, 3, 4, 5 or 6; * denotes the point of attachment of the carboxylate with the radical R;
# denotes the possible points of attachment of the carboxylate the metal ion;
R represents:
(i) a C₁₋₁₂ alkyl, C₂₋₁₂ alkene or C₂₋₁₂ alkyne radical;
(ii) a fused or confused, monocyclic or polycyclic aryl radical containing 6 to 60 carbon atoms;
(iii) a fused or nonfused, monocyclic or polycyclic heteroaryl containing 1 to 50 carbon atoms;
(iv) an organic radical comprising a metal element chosen from the group comprising ferrocene, porphyrin and phthalocyanin;
the R radical being optionally substituted with one or more R² groups, independently chosen from the croup comprising alkyl: C₂₋₁₀ alkene; C₂₋₁₀ alkyne; C₃₋₁₀ cycloalkyl: heteroalkyl: C₁₋₁₀ haloalkyl; C₆₋₁₀ aryl; C₃₋₂₀ heterocyclic; (C₁₋₁₀)alkyl(C₆₋₁₀)aryl; (C₁₋₁₀)alkyl(C₃₋₁₀)heteroaryl; F; Cl; Br; I; —NO₂; —CN; —CF₃; —CH₂CF₃; —OH; —CH₂OH; —CH₂CH₂OH; —NH₂; —CH₂NH₂; —NHCHO; —COOH; —CONH₂; —SO₃H; —CH₂SO₂CH₃; —PO₃H₂; or a -GR^{G1} function in which G is —O—, —S—, —NR^{G2}—, —C(═O)—, —S(═O)—, —SO₂—, —C(═O)O—, —C(═O)NR^{G2}—, —OC(═O)O—, —NR^{G2}C(═O)—, —OC(═O)O—, —OC(═O)NR^{G2}—, —NR^{G2}C(═O)O—, —NR^{G2}C(═O)NR^{G2}— or —C(═S)—, where each occurrence of R^{G2} is, independently of the other occurrences of R^{G2}, a hydrogen atom; or a linear, branched or cyclic, optionally substituted, C₁₋₁₂ alkyl, C₁₋₁₂ heteroalkyl, C₂₋₁₀ alkene or C₂₋₁₀ alkyne function; or a C₆₋₁₀ aryl, C₃₋₁₀ heteroaryl, C₅₋₁₀ heterocyclic, (C₁₋₁₀)alkyl(C₆₋₁₀)aryl or (C₁₋₁₀)alkyl(C₃₋₁₀)heteroaryl group in which the aryl, heteroaryl or heterocyclic radical is optionally substituted: or else, when G represents —NR^{G2}—, R^{G1} and R^{G2}, together with the nitrogen atom to which they are bonded, form a heterocycle or a heteroaryl which is optionally substituted.

2. The solid according to claim 1, in which the ligand is a di-, tri-, tetra- or hexacarboxylate ligand chosen from the group comprising: fumarate, succinate: glutarate, muconate, adipate, 2,5-thiophene-dicarboxylate, terephtnalate, 2,5-pyrazinedicarboxylate, naphthalene-2,6-dicarboxylate, biphenyl-4,4′-dicarboxylate, azobenzenedicarboxylate, dichloroazobenzenedicarboxylate, azobenzenetetracarboxylate, dihydroxoazobenenedicarboxylate, benzene-1,2,4-tricarboxylate, benzene-1,3,5-tricarboxylate, benzene-1,3,5-tribenzoate, 1,3,5-tris[4′-carboxy(1,1′-biphenyl-4-yl)]benzene, benzene-1,2,4,5-tetracarboxylate; naphthalene-2,3,6,7-tetracarboxylate, naphthalene-1,4,5,8-tetracarboxylate, biphenyl-3,5,3′,5′-tetracarboxylate, and the modified analogs chosen from the group comprising 2-aminoterephthalate, 2-nitroterephthalate, 2-methylterephthalate, 2-chloroterephthalate, 2-bromoterephthalate, 2,5-dihydroxoterephthalate, tetrafluoroterephthalate, 2,5-dicarboxyterephthalate, dimethyl-4,4′-biphenyldicarboxylate, tetramethyl-4,4′-biphenyldicarboxylate and dicarboxy-4,4′-biphenyldicarboxylate.

3. The solid according to claim 1, in which the anion X is chosen from the croup comprising OH⁻, Cl⁻, F⁻, R—(COO)ₙ⁻, PF₆⁻ and ClO₄⁻ with R and n as defined in claim 1.

4. The solid according to claim 1, comprising a mass percentage of M in the dry phase of from 5% to 50%.

5. The solid according to claim 1, in which the pore size of the MOF material is from 0.4 to 6 nm.

6. The solid according to claim 1, in which the solid has a pore volume of from 0 to 4 cm³/g.

7. The sold according to claim 1, in which the solid has a gas loading capacity of from 0.5 to 50 mmol of gas per gram of dry solid.

8. The solid according to claim 1, in which at least 1 to 5 mmol of gas per gram of dry solid coordinates with M.

9. The solid according to claim 1, in which said solid; has a flexible structure which swells or contracts with an amplitude ranging from 10% to 300%.

10. The solid according to claim 1, in which said sod has a rigid structure which swells or contracts with an amplitude ranging from 0 to 10%.

11. The solid according to claim 10, in which the solid has a pore volume of from 0.5 to 4 cm³/g.

12. The solid according to claim 1, in which said solid comprises a three-dimensional succession of units corresponding to formula (I) which are chosen from the group comprising:
Fe₃OX[C₂H₂(CO₂)₂]₃ of flexible structure,
Fe₃OX[C₆H₄(CO₂)₂]₃ of flexible structure,
Fe₃OX[C₁₀H₆(CO₂)₂]₃ of flexible structure,
Fe₃OX[C₁₂H₆(CO₂)₂]₃ of flexible structure,
Fe₃OX[C₄H₄(CO₂)₂]₃ of flexible structure,
Fe₁₂O(OH)₁₈(H₂O)₃[C₆H₃(CO₂)₃]₆ of rigid structure,
Fe₃OX[C₆H₃(CO₂)₃]₂ of rigid structure.
Fe₃OX[C₆H₄(CO₂)₂]₃ of rigid structure,
Fe₆O₂X₂[C₁₀H₂(CO₂)₄]₃ of rigid structure,
in which X is as defined in claim 1 or 2.

13. The solid according to claim 1, in which the gas is NO.

14. The solid according to claim 1, comprising at its surface at least one organic surface agent.

15. The solid according to claim 14, in which the organic surface agent is chosen from the group comprising:
oligosaccharide, for instance cyclodextrins,
a polysaccharide, for instance chitosan, dextran, fucoidan, alginate, pectin, amylase: starch, cellulose or xylan,
glycosaminoglycan, for instance hyaluronic acid or heparin,
a polymer, for instance polyethylene glycol (PEG), polyvinyl alcohol or polyethyleneimine,
a surfactant, for instance pluronic or lecithin,
vitamins, for instance biotin,
coenzymes, for instance lipoic acid,
antibodies or antibody fragments,
amino adds or peptides.

16. The solid according to claim 14, in which the organic surface agent is a targeting molecule chosen from the group comprising: biotin, chitosan, lipoic acid, an antibody or antibody fragment, and a peptide.

17. A method for preparing a solid:
(i) in mixing in a polar advent:
at least one solution comprising at least one metal inorganic precursor in the form of a metal M, of a metal salt of M or of a coordination complex comprising a metal ion of M,
at least one ligand L′ comprising a radical R comprising q groups *—C(═O)—R³ in which
q and A are as defined above,
* denotes the point of attachment of the group with the radical A,
R³ is chosen from the group comprising an OH, an OY, with Y being an alkali metal cation, a halogen, or a radical —OR⁴, —O—C(═O)R⁴ or —NR⁴R^{4′}, in which R⁴ and R^{4′} are C₁₋₁₂ alkyl radicals,
so as to obtain an MOF material;
(ii) in activating the MOF material obtained in (i); and
(iii) in bringing the MOF material obtained in step (ii) into contact with a Lewis base gas, at least a part of which coordinates with M, so as to obtain said solid.

18. The method according to claim 17, in which step (ii) is also a step of reducing the metal centers M of said MOF material to give M^{z+} ions in which z is from 2 to 4.

19. The method according to claim 17, in which activation step (ii) is carried out at a temperature of from 25 to 300° C.

20. The method according to claim 17, in which activation step (ii) is carried out at a pressure of from 1 to 10⁻² Pa.

21. The method according to claim 17, in which, in step (iii) of bringing into contact, the gas is in pure form or as a mixture with an inert gas.

22. The method of preparation according to claim 17, also comprising a step (iv) of attaching at least one organic surface agent, said step being carried out during or after reaction step (i) or after activation step (ii) and before step (iii) of bringing the MOF material into contact with the gas.

23. The method according to claim 17, in which, in step (iii), the MOF material obtained in step (ii) is brought into contact with NO.

24. The method according to claim 22, in which step (iii) is carried out at a temperature of from −100° C. to +50° C.

25. The method according to claim 22, in which, step (iii) is carried out at a pressure of from 10⁵ to 10⁶ Pa.

26-32. (canceled)
